(19) United States
(12) Patent Application Publication
(10) Pub. No.: US 2003/0104945 A1
(43) Pub. Date: Jun. 5, 2003

(54) 2,4-Diamino-1,3,5-triazines, their preparation, and their use as herbicides and plant growth regulators

(76) Inventors: Wolfgang Giencke, Hofheim (DE); Lothar Willms, Hofheim (DE); Hansjorg Dietrich, Hofheim (DE); Thomas Auler, Bad Soden (DE); Hermann Bieringer, Eppstein (DE); Hubert Menne, Hofheim (DE)

Correspondence Address: FROMMER LAWRENCE & HAUG LLP, 745 Fifth Avenue, New York, NY, 10151, US

(21) Appl. No.: 10/146,700

(22) Filed: May 14, 2002

(30) Foreign Application Priority Data
May 16, 2001 (DE) 101 23 845.2

(51) Int. Cl. A01N 43/66; C 07D 4 3/02

(52) U.S. Cl. 504/230; 544/211; 544/212; 504/231

(57) ABSTRACT

Compounds of the formula (I) and their salts

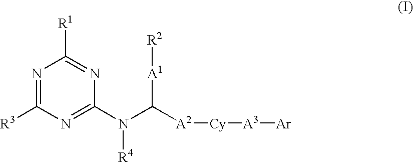

in which

R¹ to R⁴, A¹, A², A³, Cy and Ar are as defined in claim 1 are suitable as herbicides and plant growth regulators. The compounds (I) can be prepared by processes as claimed in claim 7 via novel intermediates, for example the formula (III) and (V).

The invention is in the technical field of the crop protection agents, such as herbicides and plant growth regulators, in particular of the herbicides for selectively controlling harmful plants in crops of useful plants. It is known that 6-substituted 2-amino-4-(N-phenylalkylamino)-1,3,5-triazines, which can additionally be further substituted, have herbicidal and plant-growth-regulatory properties; cf. WO-A-97/00254, WO-A-97/08156, WO-A-98/15537, WO-A-99/37627 and the literature cited in each of these.

Some of the known active substances have disadvantages on use, be it an insufficient herbicidal action against harmful plants, too narrow a weed spectrum which can be controlled with an active substance, or too little selectivity in crops of useful plants. Other active substances cannot be prepared economically on an industrial scale owing to precursors and reagents which can only be obtained with difficulty or only have insufficient chemical stability.

Object of the invention is to provide alternative active substances of the 2,4-diamino-1,3,5-triazine type which, if appropriate, can be used advantageously as herbicides or plant growth regulators.

The present invention relates to compounds of the formula (I) and their salts

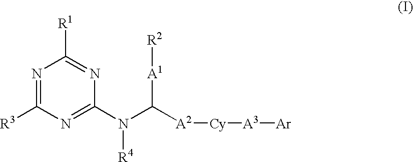

in which

R¹ is aryl, aryloxy, arylthio, arylamino, N-aryl-N—(C₁-C₄)alkylamino, (C₃-Cₚ)cycloalkyl, (C₃-C₉)cycloalkyloxy, (C₃-C₉)cycloalkylthio, (C₃-C₉)cycloalkylamino, N—(C₃-C₉)cycloalkyl-N—(C₁-C₄)alkylamino, di-[(C₃-C₉)cycloalkyl]-amino, heterocyclyl, heterocyclyloxy, heterocyclylthio, heterocyclylamino or N-heterocyclyl-N—(C₁-C₄)alkylamino, it being possible for each of the last-mentioned 16 radicals to be unsubstituted or substituted, preferably unsubstituted or substituted by one or more radicals selected from the group consisting of halogen, hydroxyl, amino, nitro, formyl, carboxyl, sulfo, cyano, thiocyanato, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₁-C₆)alkoxy, (C₁-C₆)haloalkoxy, (C₂-C₆)alkenyloxy, (C₂-C₆)haloalkenyloxy, (C₂-C₆)alkynyloxy, (C₂-C₆)haloalkynyoxy, (C₁-C₆)alkylthio, (C₁-C₆)haloalkylthio, mono(C₁-C₆)alkylamino, di(C₁-C₆)alkylamino, (C₃-C₉)cycloalkyl, (C₅-C₉)cycloalkenyl, [(C₁-C₅)alkyl]carbonyl, [(C₁-C₆)alkoxy]carbonyl, aminocarbonyl, mono(C₁-C₆)alkylaminocarbonyl, di(C₁-C₆)alkylaminocarbonyl, (C₁-C₆)alkylsulfonyl and (C₁-C₆)haloalkylsulfonyl, and, including substituents, preferably has up to 30 carbon atoms, or

hydrogen, (C₁-C₆)alkyl, (C₂-C₆)alkenyl, (C₂-C₆)alkynyl, (C₁-C₆)alkoxy, (C₂-C₆)alkenyloxy, (C₂-C₆)alkynyloxy, (C₁-C₆)alkylthio, (C₂-C₆)alkenylthio, (C₂-C₆)alkynylthio, (C₁-C₆)alkylamino or di[(C₁-C₆)alkyl]amino,

where each of the last-mentioned 11 radicals is unsubstituted or substituted by one or more radicals selected from the group consisting of halogen, hydroxyl, cyano, nitro, thiocyanato, (C₁-C₄)alkoxy, (C₁-C₄)haloalkoxy, (C₂-C₄)alkenyloxy, (C₂-C₄)haloalkenyloxy, (C₁-C₄)alkylthio, (C₁-C₄)alkylsulfinyl, (C₁-C₄)alkylsulfonyl, (C₁-C₄)haloalkylthio, (C₁-C₄)haloalkylsulfinyl, (C₁-C₄)haloalkylsulfonyl, (C₃-C₉)cycloalkyl which is unsubstituted or substituted, phenyl which is unsubstituted or substituted, heterocyclyl which is unsubstituted or substituted, and radicals of the formulae R′—C(═Z′)—, R′—C(═Z′)—Z—, R′—Z—C(═Z′)—, R′R″N—C(═Z′)—, R′—Z—C(═Z′)—O—, R′R″N—C(═Z′)—Z—, R′—Z—C(═Z′)—N R″— and R′R″N—C(═Z′)—NR′″—,

where R′, R″ and R′″ in each case independently of one another are (C₁-C₆)alkyl, aryl, aryl(C₁-C₆)alkyl, (C₃-C₉)cycloalkyl or (C₃-C₉)cycloalkyl(C₁-C₆)alkyl, where each of the last-mentioned 5 radicals is unsubstituted or substituted and in which Z and Z independently of one another are in each case an oxygen or sulfur atom,

and, including substituents, preferably has 1 to 30 carbon atoms,

R² is (C₁-C₆)alkyl, (C₂-C₆)alkenyl, (C₂-C₆)alkynyl, where each of the last-mentioned three radicals is unsubstituted or substituted, (C₃-C₉)cycloalkyl which is unsubstituted or substituted, (C₄-C₉)cycloalkenyl which is unsubstituted or substituted, heterocyclyl which is unsubstituted or substituted, or phenyl which is unsubstituted or substituted, where R² including substituents preferably has up to 30 carbon atoms,

R³ is hydrogen, (C₁-C₆)alkyl, aryl or (C₃-C₉)cycloalkyl, where each of the last-mentioned 3 radicals is unsubstituted or substituted, or is a radical of the formula —N(B¹—D¹)(B²—D²) or —NR′—N(B¹—D¹)(B²—D²), where B¹, B², D¹ and D² are in each case as defined hereinbelow and R′ is hydrogen, (C₁-C₆)alkyl or [(C₁-C₄)alkyl]carbonyl,

or is a group of the formula

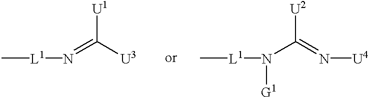

where

L¹ is a direct bond, —O—, —S— or a group of the formula —NG²—, preferably a direct bond,

U¹, U² independently of one another are a group of the formula G³, OG⁴, SG⁵, NG⁶G⁷, NG⁸NG⁹G¹⁰, NG¹¹OG¹² or NG¹¹SG¹²,

U³ is a group of the formula G¹³, OG¹⁴, SG¹⁵, NG¹⁶G¹⁷, NG¹⁸NG¹⁹G²⁰, NG²¹OG or NG ³SG²⁴,

U⁴ is a group of the formula G²⁵, OG²⁶, SG²⁷ or NG²⁸G²⁹,

where the radicals G¹ to G²⁹ independently of one another are hydrogen, aryl which is unsubstituted or substituted and including substituents preferably has 6 to 30 carbon atoms, or (C₃-C₉)cycloalkyl which is unsubstituted or substituted and including substituents preferably has 3 to 30 carbon atoms, or heterocyclyl which is substituted or unsubstituted and including substituents preferably has 2 to 30 carbon atoms, or (C₁-C₆)alkyl, (C₂-C₆)alkenyl or (C₂-C₆)alkynyl,

where each of the last-mentioned 3 radicals is unsubstituted or substituted by one or more radicals selected from the group consisting of halogen, hydroxyl, cyano, nitro, thiocyanato, (C₁-C₄)alkoxy, (C₁-C₄)haloalkoxy, (C₂-C₄)alkenyloxy, (C₂-C₄)haloalkenyloxy, (C₁-C₄)alkylthio, (C₁-C₄)alkylsulfinyl, (C₁-C₄)alkylsulfonyl, (C₁-C₄)haloalkylthio, (C₁-C₄)haloalkylsulfinyl, (C₁-C₄)haloalkylsulfonyl, (C₃-C₉)cycloalkyl which is unsubstituted or substituted, phenyl which is unsubstituted or substituted and heterocyclyl which is unsubstituted or substituted,

or the radicals U¹ and U³ or U² and U⁴ or U² and G¹ or U⁴ and G¹ pairwise with the atoms linking them are in each case a carbocyclic or heterocyclic ring with 4 to 7 ring atoms, the ring being unsubstituted or substituted,

R⁴ is a radical of the formula —B³—D³, where B³ and D³ are as defined hereinbelow and R⁴ including substituents preferably has up to 20 carbon atoms,

Ar is aryl, (C₃-C₉)cycloalkyl, (C₄-C₉)cycloalkenyl or heterocyclyl, where each of the last-mentioned 4 radicals is unsubstituted or substituted by (X)ₙ, preferably aryl including substituents has 6 to 30 carbon atoms, (C₃-C₉)cycloalkyl including substituents has 3 to 30 carbon atoms, (C₄-C₉)cycloalkenyl including substituents has 4 to 30 carbon atoms and heterocyclyl including substituents has 2 to 30 carbon atoms,

where (X)ₙ is in each case

n substituents X, where X in each case independently of one another are halogen, hydroxyl, amino, nitro, formyl, carboxyl, cyano, thiocyanato, aminocarbonyl or (C₁-C₆)alkyl, (C₁-C₆)alkoxy, (C₁-C₆)alkylthio, mono(C₁-C₆)alkylamino, di(C₁-C₄)alkylamino, (C₂-C₆)alkenyl, (C₂-C₆)alkynyl, [(C₁-C₆)alkyl]carbonyl, [(C₁-C₆)alkoxy]carbonyl, mono(C₁-C₆)alkylaminocarbonyl, di(C₁-C₄)alkylaminocarbonyl, N—(C₁-C₆)alkanoylamino, N—(C₁-C₄)alkanoyl-N—(C₁-C₄)alkylamino or N,N-di(C₁-C₄)alkanoylamino,

where each of the last-mentioned 14 radicals is unsubstituted or substituted, preferably unsubstituted or substituted by one or more radicals selected from the group consisting of halogen, hydroxyl, amino, nitro, formyl, carboxyl, cyano, thiocyanato, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, (C₃-C₉)cycloalkylamino, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, phenyl, phenoxy, phenylthio, phenylcarbonyl, phenylamino, heterocyclyl, heterocyclyloxy, heterocyclylthio and heterocyclylamino,

where each of the last-mentioned 9 radicals is unsubstituted or has one or more substituents selected from the group consisting of halogen, nitro, cyano, (C₁-C₄)alkyl, (C₁-C₄)alkoxy, (C₁-C₄)alkylthio, (C₁-C₄)haloalkyl, (C₁-C₄)haloalkoxy, (C₁-C₄)haloalkylthio, formyl, (C₁-C₄)alkylcarbonyl and (C₁-C₄)alkoxycarbonyl,

or (C₃-C₉)cycloalkyl, (C₃-C₉)cycloalkoxy, (C₃-C₉)cycloalkylamino, phenyl, phenoxy, phenylthio, phenylcarbonyl, phenylamino, heterocyclyl, heterocyclyloxy, heterocyclylthio or heterocyclylamino,

where each of the last-mentioned 12 radicals is unsubstituted or substituted, preferably unsubstituted or substituted by one or more radicals selected from the group consisting of halogen, hydroxyl, amino, nitro, formyl, carboxyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl and di(C₁-C₄)alkylaminocarbonyl,

or two adjacent radicals X together form a fused cycle with 4 to 6 ring atoms which is carbocyclic or contains hetero ring atoms selected from the group consisting of O, S and N and which is unsubstituted or substituted by one or more radicals selected from the group consisting of halogen, (C₁-C₄)alkyl and oxo, and

n is 0, 1, 2, 3, 4 or 5, preferably 0, 1, 2, 3 or 4, in particular 1 or 2,

A¹ and A² in each case independently of one another are a direct bond or straight-chain alkylene having 1 to 4 carbon atoms or straight-chain alkenylene or alkynylene having in each case 2 to 5 carbon atoms, where each of the last-mentioned three diradicals is unsubstituted or substituted by one or more radicals selected from the group consisting of halogen, nitro, cyano, thiocyanato and radicals of the formula —B⁴—D⁴, or are a divalent radical of the formula V¹, V², V³, V⁴ or V⁵,

—CR^{a}R^{b}—W*—CR^{c}R^{d}— (V¹)

—CR^{a}R^{b}—W*—CR^{c}R^{d}—CR^{e}R^{f}— (V²)

—CR^{a}R^{b}—CR^{c}R^{d}—W*—CR^{e}R^{f}— (V³)

—CR^{a}R^{b}—CR^{c}R^{d}—W*— (V⁴)

—CR^{a}R^{b}—W*— (V⁵)

where each of the radicals R^{a}, R^{b}, R^{c}, R^{d}, R^{e} and R^{f} in each case independently of one another is hydrogen, halogen, nitro, cyano, thiocyanato or a radical of the formula —B⁵—D⁵,

W* is in each case an oxygen atom, a sulfur atom or a group of the formula N(B⁶—D⁶) and

B⁴, B⁵, B⁶, D⁴, D⁵ and D⁶ are as defined hereinbelow,

A³ is a direct bond or straight-chain alkylene having 1 to 4 carbon atoms, or straight-chain alkenylene or alkynylene having in each case 2 to 5 carbon atoms, each of the last-mentioned three diradicals being unsubstituted or substituted by one or more radicals selected from the group consisting of halogen, nitro, cyano, thiocyanato and radicals of the formula —B⁷—D⁷, or is a divalent radical of the formula W¹, W², W³, W⁴, W⁵, W⁶, W⁷ or W⁸,

—CR^{aa}R^{bb}—W**—CR^{cc}R^{dd}— (W¹)

—CR^{aa}R^{bb}—W**—CR^{cc}R^{dd}—CR^{ee}R^{ff}— (W²)

—CR^{aa}R^{bb}—CR^{cc}R^{dd}—W**—CR^{ee}R^{ff}—(W³)

—CR^{aa}R^{bb}—CR^{cc}R^{dd}—W**— (W⁴)

—CR^{aa}R^{bb}—W**— (W⁵)

—W**—CR^{aa}R^{bb}—CR^{cc}R^{dd}— (W⁶)

—W**—CR^{aa}R^{bb}— (W⁷)

—W**— (W⁸)

where each of the radicals R^{aa}, R^{bb}, R^{cc}, R^{dd}, R^{ee} and R^{ff} in each case independently of one another is hydrogen, halogen, nitro, cyano, thiocyanato or a radical of the formula —B⁸—D⁸,

W** is in each case an oxygen atom, a sulfur atom or a group of the formula N(B⁹—D⁹) and

B⁷, B⁸, B⁹, D⁷, D⁸ and D⁹ are as defined hereinbelow,

B¹, B², B³, B⁶ and B⁹ in each case independently of one another are a direct bond or are a divalent group of the formulae —C(═Z*)—, —C(═Z*)—Z**—, —C(═Z*)—NH— or —C(═Z*)—NR*—, where Z* is an oxygen or sulfur atom, Z** is an oxygen or sulfur atom and R* is (C₁-C₆)alkyl, aryl, aryl(C₁-C₆)alkyl, (C₃-C₉)cycloalkyl or (C₃-C₉)cycloalkyl(C₁-C₆)alkyl, where each of the last-mentioned 5 radicals is unsubstituted or substituted and including substituents preferably has up to 20 carbon atoms,

B⁴, B⁵, B⁷ and B⁸ in each case independently of one another are a direct bond or a divalent group of the formulae —O—, —S(O)ₚ—, —S(O)ₚ—O—, —O—S(O)ₚ—, —CO—, —O—CO—, —CO—O—, —S—CO—, —CO—S—, —S—CS—, —CS—S—, —O—CO—O—, —NR^{o}—, —O—NR^{o}—, —NR^{o}—O—, —NR^{o}—CO—, —CO—NR^{o}—, —O—CO—NR^{o}— or —NR^{o}—CO—O—, where p is in each case the integer 0, 1 or 2 and R^{o} is in each case hydrogen, (C₁-C₆)alkyl, aryl, aryl(C₁-C₆)alkyl, (C₃-C₉)cycloalkyl or (C₃-C₉)cycloalkyl(C₁-C₆)alkyl, where each of the last-mentioned 5 radicals is unsubstituted or substituted and including substituents preferably has up to 20 carbon atoms,

D¹, D², D³, D⁴, D⁵, D⁶, D⁷, D⁸ and D⁹ in each case independently of one another are hydrogen, (C₁-C₆)alkyl, aryl, aryl(C₁-C₆)alkyl, (C₃-C₉)cycloalkyl or (C₃-C₉)cycloalkyl(C₁-C₆)alkyl, where each of the last-mentioned 5 radicals is unsubstituted or substituted and including substituents preferably has up to 20 carbon atoms,

Cy is a saturated carbocyclic ring with 3 to 8 ring atoms which is unsubstituted or substituted, or a saturated heterocyclic ring with 3 to 8 ring atoms and one to three hetero ring atoms selected from the group consisting of N, O, S and P, the ring being unsubstituted or substituted and, in the event of substitution on the nitrogen atom as hetero ring atom, the substituent being O or a group of the formula —B′—D′, and in the case of substitution on the hetero ring atom phosphorus, the phosphorus atom together with the substituents forming a divalent phosphorus group of the formula —P(═O)(B″—D″)— or —P(═S)(B″—D″) where B′ and B″ independently of one another are as defined under B¹ and where D′ and D″ independently of one another are as defined under D¹.

Unless specified in greater detail, divalent radicals, for example B¹=—C(═Z*)—Z**—, are defined such that, in the composite group such as, for example, —B¹—D¹, the bond of the divalent radical which, in the formula of the divalent radical is written on the right, is the one that is linked to the group D¹, i.e. —B¹—D¹ is a group of the formula —C(═Z*)—Z**—D¹; this also applies to analogous divalent radicals.

The compounds of the formula (I) can form salts by an addition reaction of a suitable inorganic or organic acid such as, for example, HCl, HBr, H₂SO₄ or HNO₃, but also oxalic acid or sulfonic acids, with a basic group such as, for example, amino or alkylamino. Suitable substituents which exist in deprotonated form, such as, for example, sulfonic acid or carboxylic acids, can form internal salts with groups which can be protonated per se, such as amino groups. Salts can also be formed by replacing, in suitable substituents such as, for example, sulfonic acid or carboxylic acids, the hydrogen by a cation which is suitable for agriculture. These salts are, for example, metal salts, in particular alkali metal salts or alkaline earth metal salts, in particular sodium salts and potassium salts, or else ammonium salts, salts with organic amines, or quaternary ammonium salts.

In formula (I) and all subsequent formulae, the radicals alkyl, alkoxy, haloalkyl, haloalkoxy, alkylamino and alkylthio and the corresponding unsaturated and/or substituted radicals in the carbon skeleton can be in each case straight-chain or branched.

The term “(C₁-C₄)alkyl” is an abbreviation of alkyl having one to 4 carbon atoms, i.e. it encompasses the radicals methyl, ethyl, 1-propyl, 2-propyl, 1-butyl, 2-butyl, 2-methylpropyl or tert-butyl. General alkyl radicals with a larger stated range of carbon atoms, for example “(C₁-C₆)alkyl” accordingly, also encompass straight-chain or branched alkyl radicals with a larger number of carbon atoms, i.e. in accordance with the example also the alkyl radicals having 5 and 6 carbon atoms. Among the hydrocarbon radicals such as alkyl, alkenyl and alkynyl radicals, also in composite radicals, the lower carbon skeletons, for example with 1 to 6 carbon atoms or, in the case of unsaturated groups, with 2 to 6 carbon atoms, are preferred, unless specified otherwise. Alkyl radicals, also in the composite areings such as alkoxy, haloalkyl and the like, are, for example, methyl, ethyl, n- or i-propyl, n-, i-, t- or 2-butyl, pentyls, hexyls, such as n-hexyl, i-hexyl and 1,3-dimethylbutyl, heptyls such as n-heptyl, 1-methylhexyl and 1,4-dimethylpentyl; alkenyl- and alkynyl radicals have the areing of the possible unsaturated radicals which correspond to the alkyl radicals; alkenyl is, for example, vinyl, allyl, 1-methyl-2-propenyl, 2-methyl-2-propenyl, 2-butenyl, pentenyl, 2-methylpentenyl or hexenyl group, preferably allyl, 1-methylprop-2-en-1-yl, 2-methylprop-2-en-1-yl, but-2-en-1-yl, but-3-en-1-yl, 1-methylbut-3-en-1-yl or 1-methylbut-2-en-1-yl.

Alkenyl includes in particular also straight-chain or branched hydrocarbon radicals with more than one double bond, such as 1,3-butadienyl and 1,4-pentadienyl, but also allenyl or cumulenyl radicals with one or more cumulated double bonds such as, for example allenyl (1,2-propadienyl), 1,2-butadienyl and 1,2,3-pentatrienyl;

Alkynyl is, for example, propargyl, but-2-yn-1-yl, but-3-yn-1-yl, 1-methylbut-3-yn-1-yl.

Alkynyl includes in particular also straight-chain or branched hydrocarbon radicals with more than one triple bond or else with one or more triple bonds and one or more double bonds, such as, for example, 1,3-butatrienyl and 3-penten-1-yn-1-yl.

Alkylidene, for example also in the form (C₁-C₁₀)alkylidene, is the radical of a straight-chain or branched alkane which is bonded via a double bond, the position of the binding side, not being stated yet. In the case of a branched alkane, only those positions are naturally suitable at which two hydrogen atoms can be replaced by the double bond; examples of radicals are ═CH₂, ═CH—CH₃, ═C(CH₃)—CH₃, ═C(CH₃)—C₂H₅ or ═C(C₂H₅)—C₂H₅.

Cycloalkyl is a carbocyclic saturated ring system with preferably 3-8 carbon atoms, for example cyclopropyl, cyclobutyl, cyclopentyl or cyclohexyl. In the case of substituted cycloalkyl, cyclic systems with substituents are encompassed, the substituents being bonded to the cycloalkyl radical by a double bond, for example an alkylidene group such as methylidene. In the case of substituted cycloalkyl, polycyclic aliphatic systems are also encompassed, such as, for example, bicyclo[1.1.0]butan-1-yl, bicyclo[1.1.0]butan-2-yl, bicyclo[2.1.0]pentan-1-yl, bicyclo[2.1.0]pentan-2-yl, bicyclo[2.1.0]pentan-5-yl, adamantan-1-yl and adamantan-2-yl.

Cycloalkenyl is a carbocyclic nonaromatic partially unsaturated ring system with preferably 4-8 carbon atoms, for example 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl. In the case of substituted cycloalkenyl, the explanations given for substituted cycloalkyl apply analogously.

Halogen is, for example, fluorine, chlorine, bromine or iodine. Haloalkyl, -alkenyl and -alkynyl are alkyl, alkenyl or alkynyl which are partially or fully substituted by identical or different halogen atoms, preferably selected from the group consisting of fluorine, chlorine and bromine, in particular from the group consisting of fluorine and chlorine, for example monohaloalkyl, perhaloalkyl, CF₃, CHF₂, CH₂F, CF₃CF₂, CH₂FCHCl, CCI₃, CHCl₂, CH₂CH₂Cl; haloalkoxy is, for example, OCF₃, OCHF₂, OCH₂F, CF₃CF₂O, OCH₂CF₃ and OCH₂CH₂Cl; the same applies analogously to haloalkenyl and other halogen-substituted radicals.

Aryl is a mono-, bi- or polycyclic aromatic system, for example phenyl, naphthyl, tetrahydronaphthyl, indenyl, indanyl, pentalenyl, fluorenyl and the like, preferably phenyl.

A heterocyclic radical or ring (heterocyclyl) can be saturated, unsaturated or heteroaromatic; unless otherwise defined, it preferably contains one or more, in particular 1, 2 or 3, hetero atoms in the heterocyclic ring, preferably selected from the group consisting of N, O, and S; it is preferably an aliphatic heterocyclyl radical with 3 to 7 ring atoms or a heteroaromatic radical with 5 or 6 ring atoms. The heterocyclic radical can be, for example, a heteroaromatic radical or ring (heteroaryl) such as, for example, a mono-, bi- or polycyclic aromatic system in which at least 1 ring contains one or more hetero atoms. It is preferably a heteroaromatic ring with a hetero atom selected from the group consisting of N, O and S, for example pyridyl, pyrrolyl, thienyl or furyl; it is furthermore preferably a corresponding heteroaromatic ring with 2 or 3 hetero atoms, for example pyrimidinyl, pyridazinyl, pyrazinyl, triazinyl, thiazolyl, thiadiazolyl, oxazolyl, isoxazolyl, pyrazolyl, imidazolyl and triazolyl. It is furthermore preferably a partially or fully hydrogenated heterocyclic radical with a hetero atom selected from the group consisting of N, O and S, for example oxiranyl, oxetanyl, oxolanyl (=tetrahydrofuryl), oxanyl, pyrrolinyl, pyrrolidyl or piperidyl.

It is furthermore preferably a partially or fully hydrogenated heterocyclic radical having 2 hetero atoms selected from the group consisting of N, O and S, for example piperazinyl, dioxolanyl, oxazolinyl, isoxazolinyl, oxazolidinyl, isoxazolidinyl and morpholinyl.

Suitable substituents for a substituted heterocyclic radical are the substituents mentioned further down, and additionally also oxo. The oxo group may also occur on those heteroring atoms which can exist at different stages of oxidation, for example in the case of N and S.

Preferred examples of heterocyclyl are a heterocyclic radical with 3 to 6 ring atoms selected from the group consisting of pyridyl, thienyl, furyl, pyrrolyl, oxiranyl, 2-oxetanyl, 3-oxetanyl, oxolanyl (=tetrahydrofuryl), pyrrolidyl, piperidyl, in particular oxiranyl, 2-oxetanyl, 3-oxetanyl or oxolanyl, or is a heterocyclic radical with two or three hetero atoms, for example pyrimidinyl, pyridazinyl, pyrazinyl, triazinyl, thienyl, thiazolyl, thiadiazolyl, oxazolyl, isoxazolyl, pyrazolyl, triazolyl, piperazinyl, dioxolanyl, oxazolinyl, isoxazolinyl, oxazolidinyl, isoxazolidinyl or morpholinyl.

When a skeleton is substituted “by one or more radicals” selected from an enumeration of radicals (=group) or a generically defined group of radicals, this includes in each case the simultaneous substitution by more than one identical and/or structurally different radicals.

Substituted radicals such as a substituted alkyl, alkenyl, alkynyl, aryl, phenyl, benzyl, heterocyclyl and heteroaryl radical are, for example, a substituted radical derived from the unsubstituted skeleton, the substituents being, for example, one or more, preferably 1, 2 or 3, radicals selected from the group consisting of halogen, alkoxy, alkylthio, hydroxyl, amino, nitro, carboxyl, cyano, azido, alkoxycarbonyl, alkylcarbonyl, formyl, carbamoyl, mono- and dialkylaminocarbonyl, substituted amino such as acylamino, mono- and dialkylamino, and alkylsulfinyl, alkylsulfonyl and, in the case of cyclic radicals, also alkyl, haloalkyl, alkylthioalkyl, alkoxyalkyl, unsubstituted or substituted mono- and dialkylaminoalkyl and hydroxyalkyl; the term “substituted radicals”, like substituted alkyl and the like, includes as substituents additionally to the abovementioned saturated hydrocarbon-containing radicals corresponding unsaturated aliphatic and aromatic radicals such as unsubstituted or substituted alkenyl, alkynyl, alkenyloxy, alkynyloxy, phenyl, phenoxy and the like. In the case of substituted cyclic radicals with aliphatic moieties in the ring, cyclic systems with substituents which are bonded to the ring by a double bond, such as those substituted by an alkylidene group such as methylidene or ethylidene, are also included.

The substituents mentioned by way of example (“first substituent level”) can, if they contain hydrocarbon-containing moieties, optionally be further substituted in these moieties (“second substituent level”), for example by one of the substituents as defined for the first substituent level. Analogous further substituent levels are possible. The term “substituted radical” preferably only comprises one or two substituent levels.

Preferred substituents for the substituent levels are, for example,

amino, hydroxyl, halogen, nitro, cyano, mercapto, carboxyl, carboxamide, SF₅, aminosulfonyl, alkyl, cycloalkyl, alkenyl, cycloalkenyl, alkynyl, monoalkylamino, dialkylamino, N-alkanoylamino, alkoxy, alkenyloxy, alkynyloxy, cycloalkoxy, cycloalkenyloxy, alkoxycarbonyl, alkenyloxycarbonyl, alkynyloxycarbonyl, aryloxycarbonyl, alkanoyl, alkenylcarbonyl, alkynylcarbonyl, arylcarbonyl, alkylthio, cycloalkylthio, alkenylthio, cycloalkenylthio, alkynylthio, alkylsulfinyl, alkylsulfonyl, monoalkyl-aminosulfonyl, dialkylaminosulfonyl, N-alkylaminocarbonyl, N,N-dialkylaminocarbonyl, N-alkanoylaminocarbonyl, N-alkanoyl-N-alkylaminocarbonyl, aryl, aryloxy, benzyl, benzyloxy, benzylthio, arylthio, arylamino and benzylamino.

In the case of radicals with carbon atoms, those with 1 to 6 carbon atoms, preferably 1 to 4 carbon atoms, in particular 1 or 2 carbon atoms, are preferred. Preferred substituents are, as a rule, those from the group consisting of halogen, for example fluorine and chlorine, (C₁-C₄)alkyl, preferably methyl or ethyl, (C₁-C₄)haloalkyl, preferably trifluoromethyl, (C₁-C₄)alkoxy, preferably methoxy or ethoxy, (C₁-C₄)haloalkoxy, nitro and cyano. Especially preferred in this context are the substituents methyl, methoxy, fluorine and chlorine. Substituted amino such as mono- or disubstituted amino is a radical selected from the group of the substituted amino radicals which are N-substituted for example by one or two identical or different radicals selected from the group consisting of alkyl, alkoxy, acyl and aryl; preferably mono- and dialkylamino, mono- and diarylamino, acylamino, N-alkyl-N-arylamino, N-alkyl-N-acylamino and N-heterocycles; preferred in this context are alkyl radicals with 1 to 4 carbon atoms; aryl is, in this context, preferably phenyl or substituted phenyl; as regards acyl in this context, the definition further below applies, preferably (C₁-C₄)alkanoyl. The same applies analogously to substituted hydroxylamino or hydrazino.

Optionally substituted phenyl is preferably phenyl which is unsubstituted or mono- or polysubstituted, preferably up to trisubstituted, by identical or different radicals selected from the group consisting of halogen, (C₁-C₄)alkyl, (C₁-C₄)alkoxy, (C₁-C₄)haloalkyl, (C₁-C₄)haloalkoxy and nitro, for example o-, m- and p-tolyl, dimethylphenyls, 2-, 3- and 4-chlorophenyl, 2-, 3- and 4-trifluoro- and -trichlorophenyl, 2,4-, 3,5-, 2,5- and 2,3-dichlorophenyl, o-, m- and p-methoxyphenyl.

Acyl is a radical of an organic acid which originates formally by removing a hydroxyl group at the acid function, it being possible for the organic radical in the acid also to be linked to the acid function via a hetero atom. Examples of acyl are the radical —CO—R of a carboxylic acid HO—CO—R, and radicals of acids derived therefrom, such as thiocarboxylic acid, optionally N-substituted iminocarboxylic acids, or the radical of carbonic monoesters, N-substituted carbamic acid, sulfonic acids, sulfinic acids, N-substituted sulfonamide acids, phosphoric acids or phosphinic acids.

Acyl is, for example, formyl, alkylcarbonyl such as [(C₁-C₄)alkyl]carbonyl, phenylcarbonyl, alkyloxycarbonyl, phenyloxycarbonyl, benzyloxycarbonyl, alkylsulfonyl, alkylsulfinyl, N-alkyl-1-iminoalkyl and other radicals of organic acids. In this context, the radicals can be further substituted in each of the alkyl or phenyl moieties, for example in the alkyl moiety by one or more radicals selected from the group consisting of halogen, alkoxy, phenyl and phenoxy; examples of substituents in the phenyl moiety are the substituents mentioned already further above in general for substituted phenyl.

Acyl is preferably an acyl radical in the strict sense, i.e. a radical of an organic acid where the acid group is linked directly to the carbon atom of an organic radical, for example formyl, alkylcarbonyl such as acetyl or [(C₁-C₄)alkyl]carbonyl, phenylcarbonyl, alkylsulfonyl, alkylsulfinyl and other radicals of organic acids.

The invention also relates to all stereoisomers encompassed by formula (I), and to their mixtures. Such compounds of the formula (I) contain one or more asymmetric carbon atoms or else double bonds, which are not stated specifically in the formula (I). The possible stereoisomers which are defined by their specific spatial shape, such as enantiomers, diastereomers, Z- and E-isomers, are all encompassed by formula (I) and can be obtained by customary methods from mixtures of the stereoisomers or else be prepared by stereoselective reactions in combination with the use of stereochemically pure starting materials.

Compounds of the abovementioned formula (I) according to the invention or their salts which are of particular interest, mainly for reasons of higher herbicidal activity, better selectivity and/or greater ease of preparation, are those in which individual radicals have one of the preferred areings which have already been mentioned or which are mentioned hereinbelow, or, in particular, those which contain a combination of one or more of the preferred areings which have already been mentioned or which are mentioned hereinbelow.

In the following text, compounds of the formula (I) according to the invention and their salts are also abbreviated to “compounds (I) according to the invention” or even further abbreviated to “compounds (I)”.

Independently of all the other radicals selected from the group consisting of R¹, R², R³, R⁴, Ar, A¹, A², A³ and Cy, including the general radicals of the corresponding subareings, and preferably in combination with preferred areings of one or more of these radicals, compounds according to the invention where the radicals in question have preferred areings as stated hereinbelow are of particular interest.

R¹ is preferably phenyl, phenoxy, phenylthio, phenylamino, N-phenyl-N—(C₁-C₄)alkylamino, where each of the last-mentioned 5 radicals unsubstituted or substituted by one or more radicals selected from the group consisting of halogen, hydroxyl, amino, nitro, formyl, carboxyl, sulfo, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, d i(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl and (C₁-C₄)haloalkylsulfonyl and, including substituents, has 6 to 30 carbon atoms, preferably 6 to 20 carbon atoms, in particular 6 to 15 carbon atoms. In this context, R¹ is, in particular, phenyl which is unsubstituted or substituted.

R¹ is preferably also (C₃-C₉)cycloalkyl which is unsubstituted or substituted by one or more radicals selected from the group consisting of halogen, hydroxyl, amino, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino and di(C₁-C₄)alkylamino and, including substituents, has 3 to 30 carbon atoms, preferably 3 to 20 carbon atoms, in particular 3 to 15 carbon atoms. In this context, R¹ is, in particular, (C₃-C₆)cycloalkyl, which is unsubstituted or substituted.

R¹ is preferably also heterocyclyl which is unsubstituted or substituted by one or more radicals selected from the group consisting of halogen, hydroxyl, amino, nitro, formyl, carboxyl, sulfonyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, d i(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl and (C₁-C₄)haloalkylsulfonyl and, including substituents, has 2 to 30 carbon atoms, preferably 2 to 20 carbon atoms, in particular 2 to 15 carbon atoms.

Here and also in the case of other radicals, heterocyclyl is as defined hereinabove, either in general terms or by way of preference.

R¹ is preferably also hydrogen or, in particular, (C₁-C₆)alkyl, (C₂-C₆)alkenyl, (C₂-C₆)alkynyl, (C₁-C₆)alkoxy or (C₁-C₆)alkylthio,

where each of the last-mentioned 5 radicals is unsubstituted or substituted by one or more radicals selected from the group consisting of halogen, hydroxyl, cyano, nitro, thiocyanato, (C₁-C₄)alkoxy, (C₁-C₄)haloalkoxy, (C₂-C₄)alkenyloxy, (C₂-C₄)haloalkenyloxy, (C₁-C₄)alkylthio, (C₁-C₄)alkylsulfinyl, (C₁-C₄)alkylsulfonyl, (C₁-C₄)haloalkylsulfinyl, (C₁-C₄)haloalkylsulfonyl and

(C₃-C₆)cycloalkyl, which is unsubstituted or substituted by one or more radicals selected from the group consisting of halogen, hydroxyl, amino, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino and di(C₁-C₄)alkylamino,

and phenyl and heterocyclyl, where each of the two last-mentioned radicals is unsubstituted or substituted by one or more radicals selected from the group consisting of halogen, hydroxyl, amino, nitro, formyl, carboxyl, sulfonyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl and (C₁-C₄)haloalkylsulfonyl, and radicals of the formula R′—C(═Z′)—, R′—C(═Z′)—Z—, R′—Z—C(═Z′)—, R′R″N—C(═Z′)—, R′—Z—C(═Z′)—O—, R′R″N—C(═Z′)—Z—, R′—Z—C(═Z′)—NR″— and R′R″N—C(═Z′)—NR′″—, where R′, R″ and R′″ in each case independently of one another are (C₁-C₄)alkyl, phenyl, phenyl(C₁-C₄)alkyl, (C₃-C₆)cycloalkyl or (C₃-C₆)cycloalkyl(C₁-C₄)alkyl, where each of the 5 last-mentioned radicals is unsubstituted or substituted by one or more radicals selected from the group consisting of halogen, hydroxyl, amino, nitro, formyl, cyano, thiocyanato, (C₁-C₄)alkoxy, (C₁-C₄)alkylthio, mono(C₁-C₄)alkylamino, di(C₁-C₄)alkylamino, (C₂-C₄)alkenyl, (C₂-C₄)alkynyl, (C₃-C₆)cycloalkyl and, in the case of cyclic radicals, also (C₁-C₄)alkyl and (C₁-C₄)haloalkyl, and where Z and Z′ independently of one another are in each case an oxygen or sulfur atom,

and, including substituents, preferably has 1 to 20 carbon atoms, in particular 1 to 15 carbon atoms;

R¹ is furthermore preferably

(C₁-C₄)alkyl, which is unsubstituted or substituted by one or more radicals selected from the group consisting of halogen, (C₁-C₄)alkoxy, (C₁-C₄)alkylthio, (C₁-C₄)alkylsulfonyl and (C₃-C₉)cycloalkyl, which is unsubstituted or substituted and

phenyl which is unsubstituted or substituted by one or more radicals selected from the group consisting of halogen, (C₁-C₄)alkyl and (C₁-C₄)haloalkyl, (C₁-C₄)alkoxy, (C₁-C₄)haloalkoxy, (C₁-C₄)alkylthio, amino, mono- and di[(C₁-C₄)alkyl]amino, (C₁-C₄)alkanoylamino, benzoylamino, nitro, cyano, [(C₁-C₄)alkyl]carbonyl, formyl, carbamoyl, mono- and di[(C₁-C₄)alkyl]aminocarbonyl and (C₁-C₄)alkylsulfonyl, and

heterocyclyl which has 3 to 6 ring atoms and 1 to 3 heteroring atoms selected from the group consisting of N, O and S, the ring being unsubstituted or substituted by one or more radicals selected from the group consisting of halogen (C₁-C₄)alkyl and oxo, or

phenyl which is unsubstituted or substituted by one or more radicals selected from the group consisting of halogen, hydroxyl, amino, nitro, formyl, carboxyl, sulfonyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl and (C₁-C₄)haloalkylsulfonyl and, including substituents, has 2 to 30 carbon atoms, preferably 2 to 20 carbon atoms, in particular 2 to 15 carbon atoms.

R¹ is furthermore preferably (C₁-C₄)alkyl, (C₁-C₄)haloalkyl, benzyl or [(C₃-C₆)cycloalkyl]-(C₁-C₂)alkyl, in particular (C₁-C₄)alkyl, (C₁-C₄)haloalkyl or [(C₃-C₆)cycloalkyl]methyl, very particularly —CH₃, —CH₂F, —CHF₂, —CF₃, —CH₂Cl, —CHCl₂, —CCl₃, —CH₂Br, —CHBr₂, —CH₂CH₃, —CH₂CH₂F, —CF₂CHF₂, —CH₂CH₂Cl, —CHFCH₃, —CHFCH₂CH₃, —CH₂CH₂Br—CH(CH₃)₂, —CF(CH₃)₂, —C(CH₃)₂Cl, —CH₂CH₂CH₂F, —CH₂CH₂CH₂Cl or cyclopropylmethyl or 1-fluorocycloprop-1-yl.

R² is preferably (C₁-C₆)alkyl, (C₂-C₆)alkenyl, (C₂-C₆)alkynyl, (C₃-C₉)cycloalkyl, (C₄-C₉)cycloalkenyl, phenyl or heterocyclyl (“Basic radicals” for R²), where each of the last-mentioned 7 radicals is unsubstituted or substituted by one or more radicals selected from the group consisting of the radicals A), B) and C), where

Group A) consists of the radicals halogen, hydroxyl, amino, nitro, formyl, carboxyl, aminocarbonyl, aminosulfonyl, sulfo, cyano, thiocyanato and oxo,

Group B) consists of the radicals (C₁-C₆)alkoxy, (C₁-C₆)alkylthio, mono(C₁-C₆)alkylamino, di(C₁-C₄)alkylamino, (C₃-C₉)cycloalkyl, (C₄-C₉)cycloalkenyl, radicals of the formulae R′—C(═Z′)—, R′—C(═Z′)—Z—, R′—Z—C(═Z′)—, R′R″N—C(═Z′)—, R′—Z—C(═Z′)—O—, R′R″N—C(═Z′)—Z—, R′—Z—C(═Z′)—NR″— and R′R″N—C(═Z′)—NR′″—, where R′, R″ and R′″ in each case independently of one another are (C₁-C₆)alkyl, phenyl, phenyl(C₁-C₆)alkyl, (C₃-C₉)cycloalkyl or (C₃-C₉)cycloalkyl(C₁-C₆)alkyl and where Z and Z′ independently of one another are in each case an oxygen or sulfur atom, and, in the case of cyclic basic radicals for R² also (C₁-C₆)alkyl, (C₂-C₆)alkenyl, (C₂-C₆)alkynyl, and, in the case of nonaromatic cyclic basic radicals for R², also (C₁-C₆)alkylidene and (C₄-C₉)cycloalkylidene,

Group C) consists of radicals as defined for group B), but where each radical is substituted by one or more radicals selected from the group consisting of halogen, hydroxyl, amino, nitro, formyl, carboxyl, sulfo, cyano, thiocyanato, aminocarbonyl, (C₁-C₄)alkoxy, (C₁-C₄)alkylthio, mono(C₁-C₄)alkylamino, di(C₁-C₄)alkylamino, (C₃-C₉)cycloalkyl, (C₄-C₉)cycloalkenyl, [(C₁-C₄)alkyl]carbonyl, [(C₁-C₄)alkoxy]carbonyl, mono(C₁-C₄)alkylaminocarbonyl, di(C₁-C₄)alkylaminocarbonyl, phenyl, phenoxy, phenylthio, phenylamino, phenylcarbonyl, heterocyclyl, heterocyclyloxy, heterocyclylthio and heterocyclylamino and, in the case of cyclic radicals B), also (C₁-C₄)alkyl, and, in the case of nonaromatic cyclic radicals B), also (C₁-C₆)alkylidene and (C₄-C₉)cycloalkylidene,

where each of the last-mentioned 22 radicals is unsubstituted or substituted by one or more radicals selected from the group consisting of halogen, nitro, cyano, (C₁-C₄)alkoxy, (C₁-C₄)alkylthio, (C₁-C₄)haloalkoxy, formyl, (C₁-C₄)alkylcarbonyl and (C₁-C₄)alkoxycarbonyl and, in the case of cyclic radicals, also (C₁-C₄)alkyl, (C₁-C₄)alkoxy-(C₁-C₄)alkyl and (C₁-C₄)haloalkyl and, in the case of nonaromatic cyclic radicals, also (C₁-C₆)alkylidene and (C₄-C₉)cycloalkylidene,

and where R² including substituents has preferably 3 to 20 carbon atoms, in particular 3 to 15 carbon atoms. Preferred as (C₃-C₉)cycloalkyl radicals are, in this context, cyclopropyl, cyclobutyl, cyclopentyl or cyclohexyl, in particular cyclopropyl, cyclobutyl or cyclopentyl. Preferred as (C₄-C₉)cycloalkenyl radicals are, in this context, 1-cyclobutenyl, 2-cyclobutenyl, 1-cyclopentenyl, 2-cyclopentenyl and 3-cyclopentenyl.

In this context, heterocyclyl is as defined further above in general terms or by way of reference.

R² is, in particular, (C₁-C₄)alkyl, (C₂-C₄)alkenyl, (C₂-C₄)alkynyl, (C₃-C₆)cycloalkyl, (C₅-C₆)cycloalkenyl, phenyl or heterocyclyl, where each of the last-mentioned 6 radicals is unsubstituted or substituted by one or more radicals selected from the group consisting of the radicals A), B) and C), where

Group A) consists of the radicals halogen and cyano,

Group B) consists of the radicals (C₁-C₄)alkoxy and (C₁-C₄)alkylthio and, in the case of cyclic radicals, also (C₁-C₄)alkyl,

Group C) consists of radicals as defined in group B), but where each radical is substituted by one or more radicals selected from the group consisting of halogen, cyano, (C₁-C₄)alkoxy, (C₁-C₄)alkylthio and (C₁-C₄)haloalkoxy,

and where R² including substituents has preferably 3 to 20 carbon atoms, in particular 3 to 15 carbon atoms.

R² is especially preferably (C₁-C₄)alkyl or (C₃-C₆)cycloalkyl, where each of the last-mentioned 6 radicals is unsubstituted or substituted by one or more radicals selected from the group of the abovementioned radicals A), B) and C), or the preferred radicals mentioned in this context.

R³ is, for example, hydrogen, (C₁-C₄)alkyl, which is unsubstituted or substituted by one more radicals selected from the group consisting of halogen, hydroxyl, amino, cyano, thiocyanato, (C₁-C₄)alkoxy, (C₁-C₄)haloalkoxy, (C₁-C₄)alkylthio, (C₁-C₄)haloalkylthio, mono(C₁-C₄)alkylamino and di(C₁-C₄)alkylamino, or is phenyl or (C₃-C₆)cycloalkyl, where each of the last-mentioned 2 radicals is unsubstituted or substituted by one or more radicals selected from the group consisting of halogen, hydroxyl, amino, nitro, formyl, carboxyl, sulfonyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylamino-carbonyl, di(C₁-C₄)alkylaminocarbonyl, (C₁-C₄)alkylsulfonyl and (C₁-C₄)haloalkylsulfonyl,

or is a radical of the formula N(B¹—D¹)(B²—D²), where B¹, B², D¹ and D² are as already defined or, preferably, as defined further below, in particular amino, or is a radical selected from the following group of radicals of the formulae

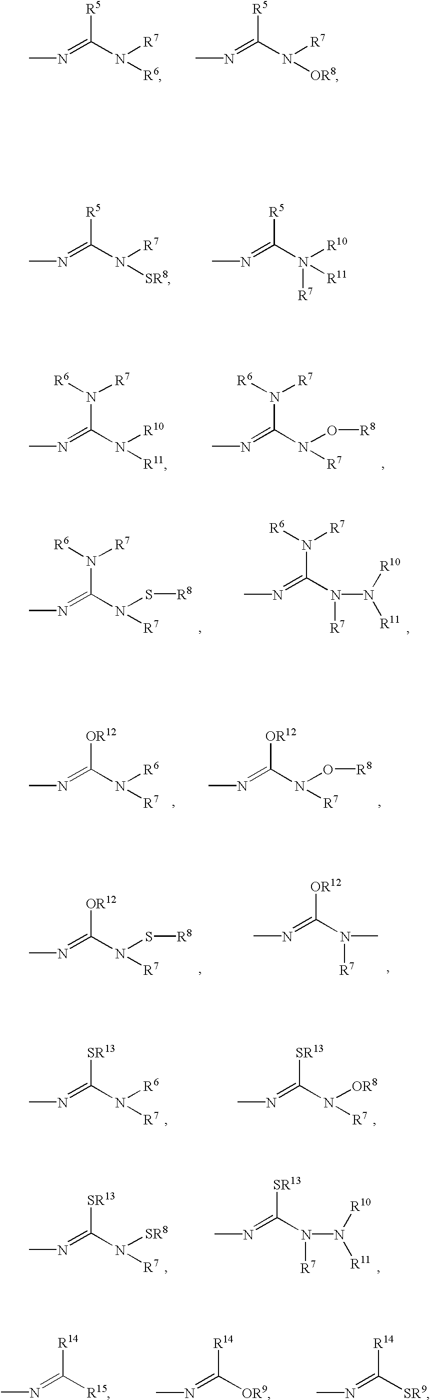

where the radicals R⁵ to R¹⁷ are defined further below.

Of particular interest are also compounds in which, in the abovementioned radicals,

R⁶ and R⁷ together with the nitrogen atom of the group NR⁶R⁷ or

R⁷ and OR⁸ together with the nitrogen atom of the group NOR⁸R⁷ or

R⁷ and SR⁸ together with the nitrogen atom of the group NSR⁸R⁷ or

R¹⁰ and R¹¹ together with the nitrogen atom of the group NR¹OR¹¹ or

OR¹² and OR13 or SR12 and SR¹³ OR⁹ or OR⁹ and R¹⁴ or SR⁹ and R14 together with the carbon atom of the respective atom group of the formula ═C(OR¹²)(OR¹³),

═C(SR¹²)(SR¹³),═C(OR⁹)(R¹⁴),═C(SR⁹)(R¹⁴) or ═C(R¹⁴)(R¹⁵) in the corresponding radical of the formulae

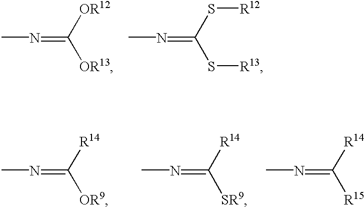

or

R⁵and R⁷together with the atom group

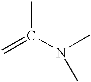

in the radicals of the formulae

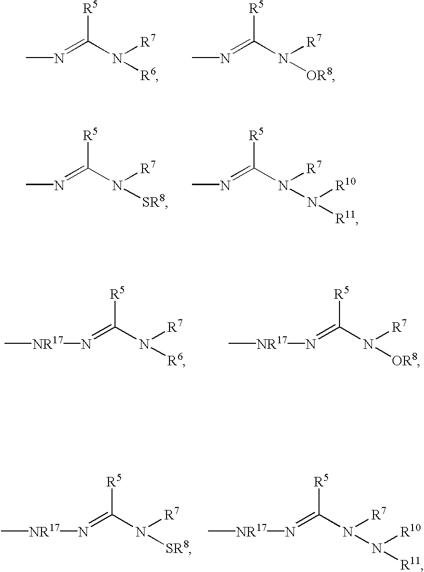

or

R⁵ and NR¹⁷ together with the carbon atom of the group of the formula

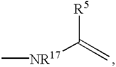

in the respective radicals or,

R⁵ and R¹⁶ together with the complete radical

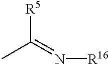

of the groups

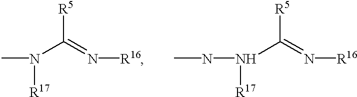

R⁷and R¹⁰ together with the atom group

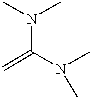

of the groups

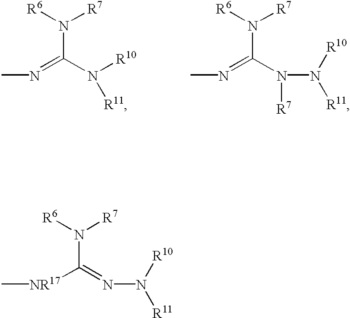

in each case independently of one another form a carbocyclic ring with 3 to 9 ring atoms or a heterocyclic ring with 3 to 7 ring atoms and 1 to 6 hetero atoms, which ring contains the abovementioned hetero atom or the abovementioned atom group, and where the optional further hetero ring atoms are selected from the group consisting of N, O and S and the carbocyclic or heterocyclic ring is in each case unsubstituted or substituted,

where the radicals R⁵ to R¹⁷ are defined further below after R⁴.

In the abovementioned atom groups, the unilaterally bound double bond “═” specifically is the binding site of a double bond or the free double bond (same areing as the binding site of a ylidene radical) and not the abbreviation of vinyl.

R⁴ for example, is a radical of the formula —B³—D³, where B³ and D³ are preferably as defined further below.

R⁴ is preferably hydrogen, (C₁-C₄)alkyl, phenyl or (C₃-C₆)cycloalkyl, where each of the last-mentioned 3 radicals is unsubstituted or substituted by one or more radicals selected from the group consisting of halogen, hydroxyl, amino, nitro, formyl, carboxyl, sulfonyl, cyano, thiocyanato, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl, (C₁-C₄)haloalkylsulfonyl and, in the case of cyclic radicals, also (C₁-C₄)alkyl and (C₁-C₄)haloalkyl, or

formyl, [(C₁-C₄)alkyl]carbonyl, [(C₁-C₄)alkoxy]carbonyl, aminocarbonyl, mono(C₁-C₄)alkylaminocarbonyl or di(C₁-C₄)alkylaminocarbonyl,

and is, in particular, hydrogen, methyl, ethyl, n-propyl or isopropyl, very preferably hydrogen.

R⁵, R⁶, R⁷, R⁸, R⁹, R¹⁰, R¹¹, R¹⁴, R¹⁵, R¹⁶ R¹⁷ in the above formulae are, independently of one another,

hydrogen, aryl which is unsubstituted or substituted and, including substituents, preferably has 6 to 30 carbon atoms, or (C₃-C₉)cycloalkyl which is unsubstituted or substituted and, including substituents, preferably has 3 to 30 carbon atoms, (C₄-C₉)cycloalkenyl which is unsubstituted or substituted and, including substituents, preferably has 4 to 30 carbon atoms, or heterocyclyl which is substituted or unsubstituted and, including substituents, preferably has 2 to 30 carbon atoms, or (C₁-C₆)alkyl, (C₂-C₆)alkenyl or (C₂-C₆)alkynyl,

where each of the last-mentioned 3 radicals is unsubstituted or substituted by one or more radicals selected from the group consisting of halogen, hydroxyl, cyano, nitro, thiocyanato, (C₁-C₄)alkoxy, (C₁-C₄)haloalkoxy, (C₂-C₄)alkenyloxy, (C₂-C₄)haloalkenyloxy, (C₁-C₄)alkylthio, (C₁-C₄)alkylsulfinyl, (C₁-C₄)alkylsulfonyl, (C₁-C₄)haloalkylsulfinyl, (C₁-C₄)haloalkylsulfonyl,

(C₃-C₉)cycloalkyl which is unsubstituted or substituted, phenyl which is unsubstituted or substituted, heterocyclyl which is unsubstituted or substituted, and radicals of the formulae R′—C(═Z′)—, R′—C(═Z′)—Z—, R′—Z—C(═Z′)—, R′R″N—C(═Z′)—, R′—Z—C(═Z′)—O—, R′R″N—C(═Z′)—Z—, R′—Z—C(═Z′)—NR″— and R′R″N—C(═Z′)—NR′″—,

where R′, R″ and R′″ in each case independently of one another are (C₁-C₆)alkyl, aryl, aryl(C₁-C₆)alkyl, (C₃-C₉)cycloalkyl or (C₃-C⁹)cycloalkyl(C₁-C₆)alkyl, where each of the 5 last-mentioned radicals is unsubstituted or substituted and where Z and Z′ independently of one another are in each case an oxygen or sulfur atom,

and, including substituents, preferably has 1 to 30 carbon atoms.

R¹², R¹³ in each case independently of one another are

aryl which is unsubstituted or substituted and, including substituents, preferably has 6 to 30 carbon atoms, or (C₃-C₉)cycloalkyl which is unsubstituted or substituted and, including substituents, preferably has 3 to 30 carbon atoms, or heterocyclyl which is substituted or unsubstituted and, including substituents, preferably has 2 to 30 carbon atoms, or

(C₁-C₆)alkyl, (C₂-C₆)alkenyl or (C₂-C₆)alkynyl, where each of the last-mentioned 3 radicals is unsubstituted or substituted by one or more radicals selected from the group consisting of halogen, hydroxyl, cyano, nitro, thiocyanato, (C₁-C₄)alkoxy, (C₁-C₄)haloalkoxy, (C₂-C₄)alkenyloxy, (C₂-C₄)haloalkenyloxy, (C₁-C₄)alkylthio, (C₁-C₄)alkylsulfinyl, (C₁-C₄)alkylsulfonyl, (C₁-C₄)haloalkylsulfinyl, (C₁-C₄)haloalkylsulfonyl, (C₃-C₉)cycloalkyl which is unsubstituted or substituted, phenyl which is unsubstituted or substituted, heterocyclyl which is unsubstituted or substituted, and radicals of the formulae R′—C(═Z′)—, R′—C(═Z′)—Z—, R′—Z—C(═Z′)—, R′R″N—C(═Z′)—, R′—Z—C(═Z′)—O—, R′R″N—C(═Z′)—Z—, R′—Z—C(═Z′)—NR″— and R′R″N—C(═Z′)—NR′″—,

where R′, R″ and R′″ in each case independently of one another are (C₁-C₆)alkyl, aryl, aryl(C₁-C₆)alkyl, (C₃-C₉)cycloalkyl or (C₃-C₉)cycloalkyl(C₁-C₆)alkyl, where each of the 5 last-mentioned radicals is unsubstituted or substituted and where Z and Z′ independently of one another are in each case an oxygen or sulfur atom,

and, including substituents, preferably has 1 to 30 carbon atoms.

Preferably, the radicals R⁵, R⁶, R⁷, R⁸, R⁹, R¹⁰, R¹¹, R¹⁴, R¹⁵, R¹⁶, R¹⁷ are in each case independently of one another hydrogen.

Preferably, the radicals R⁵, R⁶, R⁷, R⁸, R⁹, R¹⁰, R¹¹, R¹⁴, R¹⁵, R¹⁶, R¹⁷ in each case independently of one another are also

phenyl which is unsubstituted or substituted by one or more radicals selected from the group consisting of halogen, hydroxyl, amino, nitro, formyl, carboxyl, sulfo, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl and (C₁-C₄)haloalkylsulfonyl and, including substituents, have 6 to 30 carbon atoms, preferably 6 to 20 carbon atoms, in particular 6 to 15 carbon atoms.

Preferably, the radicals R⁵, R⁶, R⁷, R⁸, R⁹, R¹⁰, R¹¹, R¹⁴, R¹⁵, R¹⁶, R¹⁷ in each case independently of one another are also

(C₃-C₉)cycloalkyl which is unsubstituted or substituted by one or more radicals selected from the group consisting of halogen, hydroxyl, amino, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino and di(C₁-C₄)alkylamino and, including substituents, has 3 to 30 carbon atoms, preferably 3 to 20 carbon atoms, in particular 3 to 15 carbon atoms.

Preferably, the radicals R⁵, R⁶, R⁷, R⁸, R⁹, R¹⁰, R¹¹, R¹⁴, R¹⁵, R¹⁶, R¹⁷ in each case independently of one another are also

heterocyclyl which is unsubstituted or substituted by one or more radicals selected from the group consisting of halogen, hydroxyl, amino, nitro, formyl, carboxyl, sulfonyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl and (C₁-C₄)haloalkylsulfonyl and, including substituents, has 2 to 30 carbon atoms, preferably 2 to 20 carbon atoms, in particular 2 to 15 carbon atoms.

In this context and also in other radicals, heterocyclyl is as defined further above in general terms or by preference.

R⁵, R⁶, R⁷, R⁸, R⁹, R¹⁰, R¹¹, R¹⁴, R¹⁵, R¹⁶, R¹⁷ in each case independently of one another are preferably also (C₁-C₆)alkyl, (C₂-C₆)alkenyl or (C₂-C₆)alkynyl, where each of the last-mentioned 3 radicals is unsubstituted or substituted by one or more radicals selected from the group consisting of halogen, hydroxyl, cyano, nitro, thiocyanato, (C₁-C₄)alkoxy, (C₁-C₄)haloalkoxy, (C₂-C₄)alkenyloxy, (C₂-C₄)haloalkenyloxy, (C₁-C₄)alkylthio, (C₁-C₄)alkylsulfinyl, (C₁-C₄)alkylsulfonyl, (C₁-C₄)haloalkylsulfinyl, (C₁-C₄)haloalkylsulfonyl, (C₃-C₆)cycloalkyl which is unsubstituted or substituted by one or more radicals selected from the group consisting of halogen, hydroxyl, amino, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino and di(C₁-C₄)alkylamino, phenyl and heterocyclyl, where each of the two last-mentioned radicals is unsubstituted or substituted by one or more radicals selected from the group consisting of halogen, hydroxyl, amino, nitro, formyl, carboxyl, sulfonyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl and (C₁-C₄)haloalkylsulfonyl, and radicals of the formulae R′—C(═Z′)—, R′—C(═Z′)—Z—, R′—Z—C(═Z′)—, R′R″N—C(═Z′)—, R′—Z—C(═Z′)—O—, R′R″N—C(═Z′)—Z—, R′—Z—C(═Z′)—NR″— and R′R″N—C(═Z′)—NR′″—,

where R′, R″ and R′″ in each case independently of one another are (C₁-C₄)alkyl, phenyl, phenyl(C₁-C₄)alkyl, (C₃-C₆)cycloalkyl or (C₃-C₆)cycloalkyl(C₁-C₄)alkyl, where each of the 5 last-mentioned radicals is unsubstituted or substituted by one or more radicals selected from the group consisting of halogen, hydroxyl, amino, nitro, formyl, cyano, thiocyanato, (C₁-C₄)alkoxy, (C₁-C₄)alkylthio, mono(C₁-C₄)alkylamino, di(C₁-C₄)alkylamino, (C₂-C₄)alkenyl, (C₂-C₄)alkynyl, (C₃-C₆)cycloalkyl and, in the case of cyclic radicals, also (C₁-C₄)alkyl and (C₁-C₄)haloalkyl, and where Z and Z′ independently of one another are in each case an oxygen or sulfur atom,

and, including substituents, preferably has 1 to 20 carbon atoms, in particular 1 to 15 carbon atoms,

R⁵, R⁶, R⁷, R⁸, R⁹, R¹⁰, R¹¹, R¹⁴, R¹⁵, R¹⁶, R¹⁷ independently of one another are preferably (C₁-C₄)alkyl which is unsubstituted or substituted by one or more radicals selected from the group consisting of halogen, (C₁-C₄)alkoxy, (C₁-C₄)alkylthio, (C₁-C₄)alkylsulfonyl, (C₃-C₉)cycloalkyl which is unsubstituted or substituted and phenyl which is unsubstituted or substituted by one or more radicals selected from the group consisting of halogen, (C₁-C₄)alkyl and (C₁-C₄)haloalkyl, (C₁-C₄)alkoxy, (C₁-C₄)haloalkoxy, (C₁-C₄)alkylthio, amino, mono- and di[(C₁-C₄)alkyl]amino, (C₁-C₄)alkanoylamino, benzoylamino, nitro, cyano, [(C₁-C₄)alkyl]carbonyl, formyl, carbamoyl, mono- and di[(C₁-C₄)alkyl]aminocarbonyl and (C₁-C₄)alkylsulfonyl, and heterocyclyl with 3 to 6 ring atoms and 1 to 3 hetero ring atoms selected from the group consisting of N, O and S, where the ring is unsubstituted or substituted by one or more radicals selected from the group consisting of halogen, (C₁-C₄)alkyl and oxo, or phenyl which is unsubstituted or substituted by one or more radicals selected from the group consisting of halogen, hydroxyl, amino, nitro, formyl, carboxyl, sulfonyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl and (C₁-C₄)haloalkylsulfonyl and, including substituents, has 2 to 30 carbon atoms, preferably 2 to 20 carbon atoms, in particular 2 to 15 carbon atoms.

R¹², R¹³ in each case independently of one another are preferably also phenyl which is unsubstituted or substituted by one or more radicals selected from the group consisting of halogen, hydroxyl, amino, nitro, formyl, carboxyl, sulfo, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl and (C₁-C₄)haloalkylsulfonyl and, including substituents has 6 to 30 carbon atoms, preferably 6 to 20 carbon atoms, in particular 6 to 15 carbon atoms.

R¹², R¹³ in each case independently of one another are preferably also (C₃-C₉)cycloalkyl which is unsubstituted or substituted by one or more radicals selected from the group consisting of halogen, hydroxyl, amino, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino and di(C₁-C₄)alkylamino and, including substituents, have 3 to 30 carbon atoms, preferably 3 to 20 carbon atoms, in particular 3 to 15 carbon atoms.

R¹², R¹³ in each case independently of one another are preferably also heterocyclyl which is unsubstituted or substituted by one or more radicals selected from the group consisting of halogen, hydroxyl, amino, nitro, formyl, carboxyl, sulfonyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl and (C₁-C₄)haloalkylsulfonyl and, including substituents, has 2 to 30 carbon atoms, preferably 2 to 20 carbon atoms, in particular 2 to 15 carbon atoms.

In this context, heterocyclyl is as defined further above in general terms or by preference.

R¹², R¹³ in each case independently of one another are preferably (C₁-C₆)alkyl, (C₂-C₆)alkenyl or (C₂-C₆)alkynyl, where each of the last-mentioned 3 radicals is unsubstituted or substituted by one or more radicals selected from the group consisting of halogen, hydroxyl, cyano, nitro, thiocyanato, (C₁-C₄)alkoxy, (C₁-C₄)haloalkoxy, (C₂-C₄)alkenyloxy, (C₂-C₄)haloalkenyloxy, (C₁-C₄)alkylthio, (C₁-C₄)alkylsulfinyl, (C₁-C₄)alkylsulfonyl, (C₁-C₄)haloalkylsulfinyl, (C₁-C₄)haloalkylsulfonyl, (C₃-C₆)cycloalkyl, which is unsubstituted or substituted by one more radicals selected from the group consisting of halogen, hydroxyl, amino, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino and di(C₁-C₄)alkylamino, or phenyl and heterocyclyl, where each of the two last-mentioned radicals is unsubstituted or substituted by one or more radicals selected from the group consisting of halogen, hydroxyl, amino, nitro, formyl, carboxyl, sulfonyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl and (C₁-C₄)haloalkylsulfonyl, and radicals of the formulae R′—C(═Z′)—, R′—C(═Z′)—Z—, R′—Z—C(═Z′)—, R′R″N—C(═Z′)—, R′—Z—C(═Z′)—O—, R′R″N—C(═Z′)—Z—, R′—Z—C(═Z′)—NR″— and R′R″N—C(═Z′)—NR′″—,

where R′, R″ and R′″ in each case independently of one another are (C₁-C₄)alkyl, phenyl, phenyl(C₁-C₄)alkyl, (C₃-C₆)cycloalkyl or (C₃-C₆)cycloalkyl(C₁-C₄)alkyl, where each of the last-mentioned 5 radicals is unsubstituted or substituted by one or more radicals selected from the group consisting of halogen, hydroxyl, amino, nitro, formyl, cyano, thiocyanato, (C₁-C₄)alkoxy, (C₁-C₄)alkylthio, mono(C₁-C₄)alkylamino, di(C₁-C₄)alkylamino, (C₂-C₄)alkenyl, (C₂-C₄)alkynyl, (C₃-C₆)cycloalkyl and, in the case of cyclic radicals, also (C₁-C₄)alkyl and (C₁-C₄)haloalkyl, and where Z and Z′ independently of one another are in each case an oxygen or sulfur atom,

and, including substituents, preferably has 1 to 20 carbon atoms, in particular 1 to 15 carbon atoms.

R¹², R¹³ are in each case independently of one another preferably (C₁-C₄)alkyl which is unsubstituted or substituted by one or more radicals selected from the group consisting of halogen, (C₁-C₄)alkoxy, (C₁-C₄)alkylthio, (C₁-C₄)alkylsulfonyl, (C₃-C₉)cycloalkyl which is unsubstituted or substituted and phenyl which is unsubstituted or substituted by one or more radicals selected from the group consisting of halogen, (C₁-C₄)alkyl and (C₁-C₄)haloalkyl, (C₁-C₄)alkoxy, (C₁-C₄)haloalkoxy, (C₁-C₄)alkylthio, amino, mono- and di[(C₁-C₄)alkyl]amino, (C₁-C₄)alkanoylamino, benzoylamino, nitro, cyano, [(C₁-C₄)alkyl]carbonyl, formyl, carbamoyl, mono- and di[(C₁-C₄)alkyl]aminocarbonyl and (C₁-C₄)alkylsulfonyl, and heterocyclyl with 3 to 6 ring atoms and 1 to 3 hetero ring atoms selected from the group consisting of N, O and S, where the ring is unsubstituted or substituted by one or more radicals selected from the group consisting of halogen, (C₁-C₄)alkyl and oxo, or are phenyl which is unsubstituted or substituted by one or more radicals selected from the group consisting of halogen, hydroxyl, amino, nitro, formyl, carboxyl, sulfonyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, d i(C₁-C₄)alkylaminocarbonyl, (C₁-C₄)alkylsulfonyl and (C₁-C₄)haloalkylsulfonyl, including substituents, has 2 to 30 carbon atoms, preferably 2 to 20 carbon atoms, in particular 2 to 15 carbon atoms.

Ar is preferably phenyl, (C₃-C₉)cycloalkyl, (C₄-C₉)cycloalkenyl or heterocyclyl, where each of the last-mentioned 4 radicals is in each case unsubstituted or substituted by (X)ₙ. In particular, Ar is phenyl which is unsubstituted or substituted by (X)ₙ.

(X)ₙ is n substituents X, where substituents X preferably in each case independently of one another are halogen, hydroxyl, amino, nitro, formyl, carboxyl, cyano, thiocyanato, aminocarbonyl or (C₁-C₄)alkyl, (C₁-C₄)alkoxy, (C₁-C₄)alkylthio, mono(C₁-C₄)alkylamino, di(C₁-C₄)alkylamino, (C₂-C₄)alkenyl, (C₂-C₄)alkynyl, [(C₁-C₄)alkyl]carbonyl, [(C₁-C₄)alkoxy]carbonyl, mono(C₁-C₄)alkylaminocarbonyl, di(C₁-C₄)alkylaminocarbonyl, N—(C₁-C₆)alkanoylamino or N—(C₁-C₄)alkanoyl-N—(C₁-C₄)alkylamino,

where each of the last-mentioned 13 radicals is unsubstituted or substituted, preferably unsubstituted or substituted by one or more radicals selected from the group consisting of halogen, hydroxyl, amino, cyano, thiocyanato, (C₁-C₄)alkoxy, (C₁-C₄)haloalkoxy, (C₁-C₄)alkylthio, mono(C₁-C₄)alkylamino, di(C₁-C₄)alkylamino, (C₃-C₆)cycloalkyl, (C₃-C₆)cycloalkylamino, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, phenyl, phenoxy, phenylthio, phenylcarbonyl, heterocyclyl, heterocyclyloxy, heterocyclylthio and heterocyclylamino,

where each of the last-mentioned 8 substituents is unsubstituted or has one or more substituents selected from the group consisting of halogen, nitro, cyano, (C₁-C₄)alkyl, (C₁-C₄)alkoxy, (C₁-C₄)alkylthio, (C₀-C₄)haloalkyl, (C₁-C₄)haloalkoxy, formyl, (C₁-C₄)alkylcarbonyl and (C₁-C₄)alkoxycarbonyl,

or (C₃-C₉)cycloalkyl, phenyl, phenoxy, phenylthio, phenylcarbonyl, heterocyclyl, heterocyclyloxy, heterocyclylthio or heterocyclylamino,

where each of the last-mentioned 9 radicals is unsubstituted or substituted, preferably unsubstituted or substituted by one or more radicals selected from the group consisting of halogen, hydroxyl, amino, nitro, formyl, carboxyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₆)cycloalkyl, [(C₁-C₄)alkyl]carbonyl, [(C₁-C₄)alkoxy]carbonyl, aminocarbonyl, mono(C₁-C₄)alkylaminocarbonyl and di(C₁-C₄)alkylaminocarbonyl, or two adjacent radicals X together are a fused-on cycle with 4 to 6 ring atoms which is carbocyclic or contains hetero ring atoms selected from the group consisting of O, S and N and which is unsubstituted or substituted by one or more radicals selected from the group consisting of halogen, (C₁-C₄)alkyl and oxo.

In this context, n is preferably 0, 1, 2 or 3, in particular 0, 1 or 2.

(X)ₙ is preferably furthermore n substituents X, where substituents X in each case independently of one another are halogen, hydroxyl, amino, nitro, formyl, carboxyl, cyano, thiocyanato, (C₁-C₄)alkyl, cyano(C₁-C₄)alkyl, (C₁-C₄)alkoxy, (C₁-C₄)alkylamino, di[(C₁-C₄)alkyl]amino, halo(C₁-C₄)alkyl, hydroxyl(C₁-C₄)alkyl, (C₁-C₄)alkoxy(C₁-C₄)alkyl, halo(C₁-C₄)alkoxy(C₁-C₄)alkyl, (C₁-C₄)alkylthio, halo(C₁-C₄)alkylthio, (C₂-C₆)alkenyl, halo(C₂-C₆)alkenyl, (C₂-C₆)alkynyl, halo(C₂-C₆)alkynyl, (C₁-C₄)alkylamino(C₁-C₄)alkyl, di[(C₁-C₄)alkyl]amino(C₁-C₄)alkyl, (C₃-C₆)cycloalkylamino(C₁-C₄)alkyl, (C₃-C₉)cycloalkyl, heterocyclyl(C₁-C₄)alkyl with 3 to 9 ring members,

where the cyclic groups in the last-mentioned 3 radicals are unsubstituted or substituted by one or more radicals, preferably up to three radicals, selected from the group consisting of (C₁-C₄)alkyl, halogen and cyano, or

phenyl, phenoxy, phenylcarbonyl, phenylcarbonyl(C₁-C₄)alkyl, (C₁-C₄)alkoxycarbonyl(C₁-C₄)alkyl, (C₁-C₄)alkylaminocarbonyl(C₁-C₄)alkyl, (C₁-C₄)alkylcarbonyl, (C₁-C₄)alkoxycarbonyl, aminocarbonyl, (C₁-C₄)alkylaminocarbonyl, phenoxy(C₁-C₄)alkyl, phenyl(C₁-C₄)alkyl, heterocyclyl, heterocyclylamino, heterocyclyloxy, heterocyclylthio or one of the last-mentioned 16 radicals,

which is substituted in the acyclic moiety or, preferably, in the cyclic moiety by one or more radicals selected from the group consisting of halogen, nitro, cyano, (C₁-C₄)alkyl, (C₁-C₄)alkoxy, (C₁-C₄)alkylthio, (C₁-C₄)haloalkyl, (C₁-C₄)Haloalkoxy, formyl, (C₁-C₄)alkylcarbonyl, (C₁-C₄)alkoxycarbonyl, (C₁-C₄)alkoxy,

where heterocyclyl in the radicals contains in each case 3 to 9 ring atoms and 1 to 3 hetero ring atoms selected from the group consisting of N, O and S, or two adjacent radicals X together are a fused-on cycle with 4 to 6 ring atoms which is carbocyclic or contains hetero ring atoms selected from the group consisting of O, S and N and which is unsubstituted or substituted by one or more radicals selected from the group consisting of halogen, (C₁-C₄)alkyl and oxo.

(X)ₙ is especially preferably n substituents X, where substituents X in each case independently of one another are halogen, OH, NO₂, CN, SCN(C₁-C₆)alkyl, (C₁-C₆)alkoxy, (C₁-C₄)alkylcarbonyl or (C₁-C₄)alkyloxycarbonyl, where the last-mentioned four radicals are unsubstituted or substituted by halogen or (C₁-C₄)alkoxy, and very especially preferably n substituents X, where substituents X in each case independently of one another are halogen, hydroxyl, (C₁-C₄)alkyl or (C₁-C₄)alkoxy.

A¹ and A² independently of one another are preferably in each case a direct bond or a group of the formula —CH₂—, —CH₂CH₂—, —CH₂CH₂CH₂— or —CH₂CH₂CH₂CH₂, where each of the 4 last-mentioned diradicals is unsubstituted or substituted by one or more radicals selected from the group consisting of halogen, nitro, cyano, thiocyanato and radicals of the formula —B⁴—D⁴, or a divalent radical of the formula V¹, V², V³, V⁴ or V⁵,

—CR^{a}R^{b}—W*—CR^{c}R^{d}— (V¹)

—CR^{a}R^{b}—W*—CR^{c}R^{d}—CR^{e}R^{f}— (V²)

—CR^{a}R^{b}—CR^{c}R^{d}—W*— (V⁴)

—CR^{a}R^{b}—W*— (V⁵)

where each of the radicals R^{a}, R^{b}, R^{c}, R^{d}, R^{e} and R^{f} in each case independently of one another is hydrogen, halogen, nitro, cyano, thiocyanato or a radical of the formula —B⁵—D⁵,

W* is in each case an oxygen atom, a sulfur atom or a group of the formula N(B⁶—D⁶) and

B⁴, B⁵, B⁶, D⁴, D⁵ and D⁶ have the abovementioned meaning.

A¹ and A² independently of one another are especially preferably in each case a direct bond or a group of the formula —CH₂—, —CH₂CH₂—, —CH₂CH₂CH₂—, —CH₂CH₂CH₂CH₂, —CH₂—O—CH₂—, —CH₂—O—CH₂—CH₂—, —CH₂—CH₂—O—CH₂—, —CH₂—S—CH₂—, —CH₂—S—CH₂—CH₂—, —CH₂—CH₂—S—CH₂—, —CH₂—N H—CH₂—, —CH₂—NH—CH₂—CH₂—, —CH₂—CH₂—NH—CH₂—, —CH₂—N(CH₃)—CH₂—, —CH₂—N(CH₃)—CH₂—CH₂— or —CH₂—CH₂—N(CH₃)—CH₂-—.

A³ is preferably a direct bond or a group of the formula —O—, —S—, —OCH₂—, —SCH₂—, —CH₂O—, —CH₂S—, —CH₂—, —CH₂CH₂—, —CH₂CH₂CH₂— or —CH₂CH₂CH₂CH₂—, where each of the four last-mentioned diradicals is unsubstituted or substituted by one or more radicals selected from the group consisting of halogen, nitro, cyano, thiocyanato and radicals of the formula —B⁷—D⁷, or a divalent radical of the formula W¹, W², W³, W⁴, W⁵, W⁶, or W⁸,

—CR^{aa}R^{bb—W**—CR}^{cc}R^{dd}— (W¹)

—CR^{aa}R^{bb}—W**—CR^{cc}R^{dd}—CR^{ee}R^{ff}— (W²)

CR^{aa}R^{bb}—CR^{cc}R^{dd}—W**—CR^{ee}R^{ff}— (W³)

—CR^{aa}R^{bb}—CR^{cc}R^{dd}—W**— (W⁴)

—CR^{aa}R^{bb}—W**— (W⁵)

—W**—CR^{aa}R^{bb}—CR^{cc}R^{dd}— (W⁶)

—W**—CR^{aa}R^{bb}— (W⁷)

—W**— (W⁸)

where each of the radicals R^{aa}, R^{bb}, R^{cc}, R^{dd}, R^{ee} and R^{ff} in each case independently of one another is hydrogen, halogen, nitro, cyano, thiocyanato or a radical of the formula —B⁸—D⁸,

W** is in each case an oxygen atom, a sulfur atom or a group of the formula N(B⁹—D⁹), and

B⁷, B⁸, B⁹, D⁷, D⁸ and D⁹ are as defined below.

A³ is especially preferably a direct bond or a group of the formula —O—, —S—, —CH₂—, —CH₂CH₂—, —CH₂CH₂CH₂—, —CH₂CH₂CH₂CH₂—, —O—CH₂—, —CH₂—O—, —SCH₂— or —CH₂—S—, in particular a direct bond or —O—, —S—, —OCH₂—, —SCH₂—, —CH₂O—, —CH₂S—, —CH₂—, —CH₂CH₂— or —CH₂CH₂CH₂—.

B¹, B², B³, B⁶ and B⁹ independently of one another are preferably in each case a direct bond or a divalent group of the formulae —C(═Z*)—, —C(═Z*)—Z**—, —C(═Z*)—NH— or —C(═Z*)—NR*—, where Z* is an oxygen or sulfur atom, Z** an oxygen or sulfur atom and R* (C₁-C₆)alkyl, aryl, aryl(C₁-C₆)alkyl, (C₃-C₉)cycloalkyl or (C₃-C₉)cycloalkyl(C₁-C₆)alkyl, where each of the 5 last-mentioned radicals is unsubstituted or substituted by one or more radicals selected from the group consisting of halogen, hydroxyl, amino, nitro, formyl, carboxyl, sulfo, cyano, thiocyanato, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl, (C₁-C₄)haloalkylsulfonyl and, in the case of cyclic radicals, also (C₁-C₄)alkyl and (C₁-C₄)haloalkyl and, including substituents, preferably have up to 20 carbon atoms.

B¹, B², B³, B⁶ and B⁹ independently of one another are furthermore preferably in each case a direct bond or a divalent group of the formula —C(═Z*)—, —C(═Z*)—Z**—, —C(═Z*)—NH— or —C(═Z*)—NR*—,

where Z*═O or S, Z**═O or S and R*═(C₁-C₄)alkyl, phenyl, phenyl(C₁-C₄)alkyl, (C₃-C₆)cycloalkyl or (C₃-C₆)cycloalkyl(C₁-C₄)alkyl, where each of the 5 last-mentioned radicals is unsubstituted or substituted by one or more radicals selected from the group consisting of halogen, hydroxyl, amino, formyl, (C₁-C₄)alkoxy, (C₁-C₄)haloalkoxy, (C₁-C₄)alkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl and, in the case of cyclic radicals, also (C₁-C₄)alkyl and (C₁-C₄)haloalkyl,

Z* is in particular an oxygen atom and R* is in particular (C₁-C₄)alkyl, (C₃-C₆)cycloalkyl, phenyl or phenyl(C₁-C₄)alkyl, where each of the two last-mentioned radicals is unsubstituted or substituted in the phenyl moiety by one or more radicals selected from the group consisting of halogen, (C₁-C₄)alkyl, (C₁-C₄)haloalkyl, (C₁-C₄)alkoxy or (C₁-C₄)haloalkoxy.

B⁴, B⁵, B⁷ and B⁸ independently of one another are preferably in each case a direct bond or a divalent group of the formulae —O—, —S(O)ₚ—, —S(O)ₚ—O—, —O—S(O)ₚ—, —CO—, —O—CO—, —CO—O—, —S—CO—, —CO—S—, —S—CS—, —CS—S—, —O—CO—O—, —NR^{o}—, —O—NR^{o}—, —NR^{o}—O—, —NR^{o}—CO—, —CO—NR^{o}—, —O—CO—NR^{o}— or —NR^{o}—CO—O—, where p is the integer 0, 1 or 2 and R^{o} is hydrogen, (C₁-C₆)alkyl, aryl, aryl(C₁-C₆)alkyl, (C₃-C₉)cycloalkyl or (C₃-C₉)cycloalkyl(C₁-C₆)alkyl, where each of the 5 last-mentioned radicals is unsubstituted or substituted and, including substituents, preferably has up to 20 carbon atoms.

B⁴, B⁵, B⁷ and B⁸ independently of one another are furthermore preferably in each case a direct bond or a divalent group of the formulae —O—, —S(O)ₚ—, —CO—, —O—CO—, —CO—O— —S—CO—, —CO—S—, —NR^{o}—, —NR^{o}—CO—, —CO—NR^{o}—, —O—CO—NR^{o}— or —NR^{o}—CO—O—, where p is the integer 0, 1 or 2, and, in particular 0 or 2, and R^{o} has the abovementioned areing and is very particularly H or (C₁-C₄)alkyl.

D¹, D², D³, D⁴, D⁵, D⁶, D⁷, D⁸ and D⁹ independently of one another are in each case hydrogen, (C₁-C₆)alkyl, aryl, aryl(C₁-C₆)alkyl, (C₃-C₉)cycloalkyl or (C₃-C₉)cycloalkyl(C₁-C₆)alkyl, where each of the 5 last-mentioned radicals is unsubstituted or substituted by one or more radicals selected from the group consisting of halogen, hydroxyl, amino, nitro, formyl, carboxyl, sulfo, cyano, thiocyanato, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyi]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl, (C₁-C₄)haloalkylsulfonyl and, in the case of cyclic radicals, also (C₁-C₄)alkyl and (C₁-C₄)haloalkyl and, including substituents, preferably has up to 20 carbon atoms.

D¹, D², D³, D⁴, D⁵, D⁶, D7, D⁸ and D⁹ independently of one another are furthermore preferably in each case hydrogen, (C₁-C₄)alkyl, phenyl, phenyl(C₁-C₄)alkyl, (C₃-C₆)cycloalkyl or (C₃-C₆)cycloalkyl(C₁-C₄)alkyl, where each of the 5 last-mentioned radicals is unsubstituted or substituted by one or more radicals selected from the group consisting of halogen, hydroxyl, amino, formyl, (C₁-C₄)alkoxy, (C₁-C₄)haloalkoxy, (C₁-C₄)alkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl and, in the case of cyclic radicals, (C₁-C₄)alkyl and (C₁-C₄)haloalkyl,

and are, in particular,

hydrogen, (C₁-C₄)alkyl or (C₃-C₆)cycloalkyl or phenyl or phenyl(C₁-C₄)alkyl, where each of the two last-mentioned radicals is unsubstituted in the phenyl moiety or substituted by one or more radicals selected from the group consisting of halogen, (C₁-C₄)alkyl, (C₁-C₄)haloalkyl, (C₁-C₄)alkoxy or (C₁-C₄)haloalkoxy.

Cy is preferably a divalent (C₃-C₉)cycloalkylene group which is unsubstituted or substituted by one or more radicals selected from the group consisting of the radicals A^{c}), B^{c}) and C^{c}), where

Group A^{c}) consists of the radicals halogen, hydroxyl, amino, nitro, formyl, carboxyl, aminocarbonyl, aminosulfonyl, sulfo, cyano, thiocyanato and oxo,

Group B^{c}) consists of the radicals (C₁-C₆)alkyl, (C₂-C₆)alkenyl, (C₂-C₆)alkynyl, (C₁-C₆)alkylidene, (C₄-C₉)cycloalkylidene(C₁-C₆)alkoxy, (C₁-C₆)alkylthio, mono(C₁-C₆)alkylamino, di(C₁-C₄)alkylamino, (C₃-C₉)cycloalkyl, (C₄-C₉)cycloalkenyl, radicals of the formulae R′—C(═Z′)—, R′—C(═Z′)—Z—, R′—Z—C(═Z′)—, R′R″N—C(═Z′)—, R′—Z—C(═Z′)—O—, R′R″N—C(═Z′)—Z—, R′—Z—C(═Z′)—NR″— and R′R″N—C(═Z′)—NR′″—,

where R′, R″ and R′″ in each case independently of one another are (C₁-C₆)alkyl, phenyl, phenyl(C₁-C₆)alkyl, (C₃-C₉)cycloalkyl or (C₃-C₉)cycloalkyl(C₁-C₆)alkyl and where Z and Z′ independently of one another are in each case an oxygen or sulfur atom,

Group C^{c}) consists of radicals as defined for group B^{c}), but where each radical is substituted by one or more radicals selected from the group consisting of halogen, hydroxyl, amino, nitro, formyl, carboxyl, sulfo, cyano, thiocyanato, aminocarbonyl, (C₁-C₄)alkoxy, (C₁-C₄)alkylthio, mono(C₁-C₄)alkylamino, di(C₁-C₄)alkylamino, (C₃-C₉)cycloalkyl, (C₄-C₉)cycloalkenyl, [(C₁-C₄)alkyl]carbonyl, [(C₁-C₄)alkoxy]carbonyl, mono(C₁-C₄)alkylamino-carbonyl, di(C₁-C₄)alkylaminocarbonyl, phenyl, phenoxy, phenylthio, phenylamino, phenylcarbonyl, heterocyclyl, heterocyclyloxy, heterocyclylthio and heterocyclylamino and, in the case of cyclic radicals B^{c}), also (C₁-C₄)alkyl, and, in the case of nonaromatic cyclic radicals B^{c}), also (C₁-C₆)alkylidene and (C₄-C₉)cycloalkylidene,

where each of the last-mentioned 22 radicals is unsubstituted or substituted by one or more radicals selected from the group consisting of halogen, nitro, cyano, (C₁-C₄)alkoxy, (C₁-C₄)alkylthio, (C₁-C₄)haloalkoxy, formyl, (C₁-C₄)alkylcarbonyl and (C₁-C₄)alkoxycarbonyl and, in the case of cyclic radicals, also (C₁-C₄)alkyl, (C₁-C₄)alkoxy(C₁-C₄)alkyl and (C₁-C₄)haloalkyl and, in the case of nonaromatic cyclic radicals, also (C₁-C₆)alkylidene and (C₄-C₉)cycloalkylidene,

and where Cy including substituents preferably has 3 to 20 carbon atoms, in particular 3 to 15 carbon atoms.

Preferred as (C₃-C₉)cycloalkylene groups for Cy are cyclopropylene, cyclobutylene, cyclopentylene or cyclohexylene, in particular cyclopropylene, cyclobutylene or cyclopentylene, where each of the radicals is unsubstituted or substituted by one or more radicals selected from the group consisting of the abovementioned radicals A^{c}), B^{c}) and C^{c}).

Examples of the (C₃-C₉)cycloalkylene groups are 1,2-cis-cyclopropylene, 1,2-trans-cyclopropylene, 1,1-cyclopropylene, 1,2-cis-cyclobutylene, 1,2-trans-cyclobutylene, 1,3-cis-cyclobutylene, 1,3-trans-cyclobutylene, 1,1-cyclobutylene, 1,2-cis-cyclopentylene, 1,2-trans-cyclopentylene, 1,3-cis-cyclopentylene, 1,3-trans-cyclopentylene, 1,1-cyclopentylene, 1,2-cis-cyclohexylene, 1,2-trans-cyclohexylene, 1,3-cis-cyclohexylene, 1,3-trans-cyclohexylene, 1,4-cis-cyclohexylene, 1,4-trans-cyclohexylene, 1,1-cyclohexylene.

Cy is preferably also a saturated divalent heterocyclylene group which is unsubstituted or substituted by one or more radicals selected from the group consisting of the radicals A^{c}), B^{c}) and C^{c}) as they are defined hereinabove as substituents when Cy═(C₃-C₉)cycloalkylene group.

The saturated heterocyclylene preferably has 3 to 6 ring atoms and 1 to 3 hetero ring atoms selected from the group consisting of N, O and S; it is preferably a divalent radical selected from the group consisting of oxiranylene, aziridinylene, pyrrolidinylene, 1,3-dioxolanylene, oxathiolanylene, imidazolidinylene, pyrazolidinylene, piperidinylene, 1,4-dioxanylene, morpholinylene, 1,4-dithianylene, 1,3-dioxanylene, 1,3-dithianylene, thiomorphlinylene and piperazinylene.

The present invention also relates to processes for the preparation of the compounds of the formula (I) or their salts, which comprises

a) reacting a compound of the formula (II),

R¹-Fu (II)

where Fu is the functional group selected from the group consisting of carboxylic ester, carboxylic orthoester, carboxylic acid chloride, carboxamide, carboxylic anhydride and trichloromethyl

with a compound of the formula (III) or an acid addition salt thereof

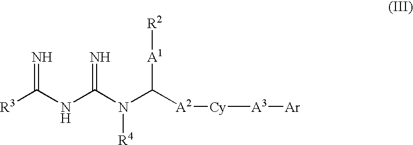

or

b) reacting a compound of the formula (IV)

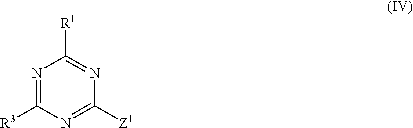

where Z¹ is an exchangeable radical or a leaving group, for example chlorine, trichloromethyl, (C₁-C₄)alkylsulfonyl, unsubstituted or substituted phenyl(C₁-C₄)alkylsulfonyl or (C₁-C₄)alkylphenylsulfonyl, with a suitable amine of the formula (V) or an acid addition salt thereof

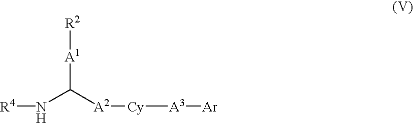

where, in formulae (II), (III), (IV) and (V), the radicals R¹, R², R³, R⁴, A¹, A², A³, Ar and Cy are as defined in formula (I).

The compounds of the formula (II) and (III) are preferably reacted with base catalysis in an inert organic solvent such as, for example, tetrahydrofuran (THF), dioxane, acetonitrile, dimethylformamide (DMF), methanol and ethanol, at temperatures of between −10° C. and the boiling point of the solvent, preferably at from 20° C. to 60° C.; when acid addition salts of the formula (III) are used, these are, as a rule, liberated in situ with the aid of a base. Suitable bases or basic catalysts are alkali metal hydroxides, alkali metal hydrides, alkali metal carbonates, alkali metal alkoxides, alkaline earth metal hydroxides, alkaline earth metal hydrides, alkaline earth metal carbonates or organic bases such as triethylamine or 1,8-diazabicyclo[5.4.0]undec-7-ene (DBU). In this context, the base in question is employed, for example, in a range of from 0.1 to 3 molar equivalents based on the compound of the formula (III). The compound of the formula (II) can be employed for example in an equimolar ratio or an excess relative to the compound of the formula (III), as a rule in a molar ratio of (III):(II) of up to 1:4, in most cases up to 1:3. In principle, the relevant processes are known from the literature (compare: Comprehensive Heterocyclic Chemistry, A. R. Katritzky, C. W. Rees, Pergamon Press, Oxford, N.Y., 1984, Vol.3; Part 2B; ISBN 0-08-030703-5, p.290).

The compounds of the formulae (IV) and (V) are preferably reacted with base catalysis in an inert organic solvent such as, for example, THF, dioxane, acetonitrile, DMF, methanol and ethanol, at temperatures of between −10° C. and the boiling point of the respective solvent or solvent mixture, preferably at from 20° C. to 60° C., where the compound (V), if employed in the form of the acid addition salts, is, if appropriate, liberated in situ with a base. Suitable bases or basic catalysts are alkali metal hydroxides, alkali metal hydrides, alkali metal carbonates, alkali metal alkoxides, alkaline earth metal hydroxides, alkaline earth metal hydrides, alkaline earth metal carbonates or organic bases such as triethylamine or 1,8-diazabicyclo[5.4.0]undec-7-ene (DBU). In this context, the base in question is, as a rule, employed in the range from 1 to 3 molar equivalents based on the compound of the formula (IV). For example, the compound of the formula (IV) can be employed in an equimolar amount relative to the compound of the formula (V) or in an excess of up to 2 molar equivalents. In principle, the relevant processes are known from the literature (compare: Comprehensive Heterocyclic Chemistry, A. R. Katritzky, C. W. Rees, Pergamon Press, Oxford, N.Y., 1984, Vol.3; Part 2B; ISBN 0-08-030703-5, p.482).

The starting materials of the formulae (II), (III), (IV) and (V) are either commercially available or can be prepared by, or analogously to, processes known from the literature. The compounds of the formula (III) and (V) are novel and also subject matter of the invention. The compounds can be prepared for example also by one of the processes described hereinbelow.

For example, the compound of the formula (IV), or a direct precursor thereof, can be prepared as follows:

1. Reacting a compound of the formula (II) with an amidino thio urea derivative of the formula (VI),

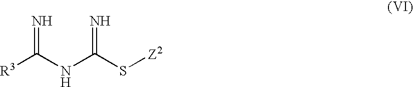

where Z² is (C₁-C₄)alkyl or phenyl(C₁-C₄)alkyl and R³ is as defined in formula (I) gives compounds of the formula (IV) in which Z¹ is —SZ².

2. Reacting an amidine of the formula (VII) or an acid addition salt thereof

H₂N—CR¹═NH (VI)

where R¹ is as defined in formula (I),

with an N-cyanodithioiminocarbonate of the formula (VII)

NC—N═C(S—Z³)₂ (VIII)

where Z³ is (C₁-C₄)alkyl or phenyl(C₁-C₄)alkyl, gives compounds of the formula (IV) where Z¹ is —S—Z³.

3. Reacting an alkali metal dicyanamide with a carboxylic acid derivative of the abovementioned formula (II) gives compounds of the formula (IV) in which Z¹ is NH₂,

4. Reacting trichloroacetonitrile with a nitrile of the formula (IX)

R¹—CN (IX)

where R¹ is as defined in formula (I), gives initially compounds of the formula (X)

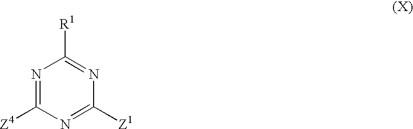

where Z¹ and Z⁴ are in each case CCl₃, and these are subsequently reacted with compounds of the formula H—R³ (R³ as in formula (I)), to give compounds of the formula (IV), where Z¹═CCl₃.

The reaction of the carboxylic acid derivatives of the formula (II) with the amidinothio urea derivatives of the formula (VI) is preferably carried out with base catalysis in an organic solvent such as, for example, acetone, THF, dioxane, acetonitrile, DMF, methanol, ethanol, at temperatures of from −10° C. to the boiling point of the solvent, preferably at from 0° C. to 20° C. However, the reaction may also be effected in water or in aqueous solvent mixtures with one or more of the abovementioned organic solvents. If the compound of the formula (VI) is employed in the form of the acid addition salts, it can, if appropriate, be liberated in situ with a base. Suitable bases or basic catalysts are alkali metal hydroxides, alkali metal hydrides, alkali metal carbonates, alkali metal alkoxides, alkaline earth metal hydroxides, alkaline earth metal hydrides, alkaline earth metal carbonates or organic bases such as triethylamine or 1,8-diazabicyclo[5.4.0]undec-7-ene (DBU). In this context, the base in question is employed for example in a range of from 1 to 3 molar equivalents based on the compound of the formula (VI). Compounds of the formula (II) and (VI) can be employed, for example, in equimolar amounts or in an excess, as a rule in a molar ratio of (III):(II) of up to 1:4, in most cases up to 1:3. In principle, the processes in question are known from the literature (cf.: H. Eilingsfeld, H. Scheuermann, Chem. Ber.; 1967, 100, 1874), while the corresponding intermediates of the formula (IV) are novel.

The reaction of the amidines of the formula (VII) with the N-cyanodithioimino-carbonates of the formula (VIII) is preferably carried out with base catalysis in an inert organic solvent such as, for example, acetonitrile, DMF, dimethylacetamide (DMA), N-methylpyrrolidone (NMP), methanol and ethanol, at temperatures of from −10° C. to the boiling point of the solvent, preferably at from 20° C. to 80° C. If the compound (VII) is employed in the form of the acid addition salts, it may, if appropriate, be liberated in situ with a base. Suitable bases or basic catalysts are alkali metal hydroxides, alkali metal hydrides, alkali metal carbonates, alkali metal alkoxides, alkaline earth metal hydroxides, alkaline earth metal hydrides, alkaline earth metal carbonates, or organic bases such as triethylamine or 1,8-diazabicyclo[5.4.0]undec-7-ene (DBU). In this context, the base in question is employed, for example, in a range of from 1 to 3 molar equivalents based on the compound of the formula (VIII), compounds of the formula (VII) and (VIII) can, as a rule, be employed in equimolar amounts or with an excess of two molar equivalents of the compound of the formula (VII). In principle, the relevant processes are known from the literature (compare: T. A. Riley, W. J. Henney, N. K. Dailey, B. E. Wilson, R. K. Robins; J. Heterocyclic Chem.; 1986, 23 (6), 1706-1714).

Intermediates of the formula (X) where Z¹=chlorine can be prepared by reacting alkali metal dicyanamide with a carboxylic acid derivative of the formula (II), in which case Fu is preferably the functional group carboxylic acid chloride or carboxamide. For example, the reactants are reacted with acid catalysis in an inert organic solvent such as, for example, toluene, chlorobenzene, or chlorinated hydrocarbons at temperatures of between −10° C. and the boiling point of the solvent, preferably at from 20° C. to 80° C., it being possible to chlorinate the resulting intermediates in situ with a suitable chlorinating reagent such as, for example, phosphorus oxychloride. Examples of suitable acids are hydrohalic acids such as HCl, or else Lewis acids such as, for example, AlCl₃ or BF₃ (cf.: U.S. Pat. No. 5,095,113, DuPont).

Intermediates of the formula (X) where Z¹, Z⁴=trihalomethyl can be prepared by reacting the corresponding trihaloacetonitriles with a carbonitrile of the formula (IX). The reactants are reacted for example with acid catalysis in an inert organic solvent such as, for example, toluene, chlorobenzene, chlorinated hydrocarbons at temperatures of between −40° C. and the boiling point of the solvent, preferably at -10° C. to 30° C. Examples of suitable acids are hydrohalic acids such as HCl or else Lewis acids such as, for example, AlCl₃ or BF₃ (cf. EP-A-130939, Ciba Geigy).

Intermediates of the formula (IV), where Z¹=(C₁-C₄)alkylmercapto or unsubstituted phenyl(C₁-C₄)alkylmercapto can be converted in an inert organic solvent such as, for example, toluene, chlorobenzene, chlorinated hydrocarbons or others at temperatures of between −40° C. and the boiling point of the solvent, preferably at from 20° C. to 80° C., with a suitable chlorinating reagent such as, for example, elemental chlorine or phosphorus oxychloride, to give more reactive chlorotriazines of the formula (IV) where Z¹=Cl (cf. J. K. Chakrabarti, D. E. Tupper; Tetrahedron 1975, 31(16), 1879-1882).

Intermediates of the formula (IV), where Z¹═(C₁-C₄)alkylmercapto or unsubstituted or substituted phenyl(C₁-C₄)alkylmercapto or (C₁-C₄)alkylphenylthio can be oxidized in a suitable solvent such as, for example, chlorinated hydrocarbons, acetic acid, water, alcohols, acetone or mixtures of these at temperatures of between 0° C. and the boiling point of the solvent, preferably at from 20° C. to 80° C., with a suitable oxidant such as, for example, n-chloroperbenzoic acid, hydrogen peroxide or potassium peroxomonosulfate (cf. T. A. Riley, W. J. Henney, N. K. Dailey, B. E. Wilson, R. K. Robins; J. Heterocyclic Chem.; 1986, 23 (6), 1706-1714).

The compounds of the formula (III) can be prepared from compounds of the formula (V) and/or their acid adducts by reaction with cyanoguamides (“dicyandiamide”), of the formula (XI),

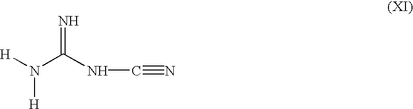

if appropriate in the presence of a reaction auxiliary such as, for example, hydrochloride and, if appropriate, in the presence of a diluent such as, for example, n-decane or 1,2-dichlorobenzene, at temperatures of, for example, between 100° C. and 200° C. (cf. EP-A-492615, preparation examples).

The amines of the formula (V) can be synthesized analogously to known methods using simple structural units as precursors. For example, the amino group can be obtained by reductively aminating suitable ketones (cf. the above-mentioned literature, for example page 1 for aminotriazine herbicides).

The following acids are suitable for preparing the acid addition salts of the compounds of the formula (i): hydrohalic acids such as hydrochloric acid or hydrobromic acid, furthermore phosphoric acid, nitric acid, sulfuric acid, mono- or bifunctional carboxylic acids and hydroxycarboxylic acids such as acetic acid, maleic acid, succinic acid, fumaric acid, tartaric acid, citric acid, salicylic acid, sorbic acid or lactic acid, and sulfonic acids such as p-toluenesulfonic acid or 1,5-naphthalenedisulfonic acid. The acid adducts of the formula (I) can be obtained in a simple fashion using the customary salt formation methods, for example by dissolving a compound of the formula (I) in a suitable organic solvent such as, for example, methanol, acetone, methylene chloride or petroleum ether, and adding the acid at temperatures of from 0° C. to 100° C., and isolated in the known manner, for example by filtration and, if appropriate, purified by washing with an inert organic solvent.

The base addition salts of the compounds of the formula (I) are preferably prepared in inert polar solvents such as, for example, water, methanol or acetone, at temperatures of from 0° C. to 100° C. Examples of bases which are suitable for preparing the salts according to the invention are alkali metal carbonates such as potassium carbonate, alkali metal and alkaline earth metal hydroxides, for example NaOH or KOH, alkali metal and alkaline earth metal hydride, for example NaH, alkali metal and alkaline earth metal alkoxides, for example sodium methoxide, potassium tert-butoxide, or ammonia or ethanolamine. Quaternary ammonium salts can be prepared for example by salt exchange or by condensation with quaternary ammonium salts of the formula [NRR′R″R′″]⁺X⁻, where R, R′, R″ and R′″ independently of one another are (C₁-C₄)alkyl, phenyl or benzyl and X is an anion, for example Cl⁻ or OH⁻.

The term “inert solvents” used in the above process variants refers in each case to solvents which are inert under the reaction conditions in question, but which need not be inert under any reaction conditions.

The compounds of the formula (I) according to the invention and their salts have an outstanding herbicidal activity against a broad spectrum of economically important monocotyledonous and dicotyledonous harmful plants. The active ingredients also act efficiently on perennial weeds which produce shoots from rhizomes, rootstocks or other perennial organs and which are difficult to control. In this context, it does not matter whether the substances are applied before sowing, pre-emergence or post-emergence.

Specific examples may be mentioned of some representatives of the monocotyledonous and dicotyledonous weed flora which can be controlled by the compounds according to the invention, without the enumeration being a restriction to certain species.

Examples of weed species on which the herbicidal compositions act efficiently are, from amongst the monocotyledonous weed species, Avena, Lolium, Alopecurus, Phalaris, Echinochloa, Digitaria, Setaria and Cyperus species, from the annual group, and, among the perennial species, Agropyron, Cynodon, Imperata and Sorghum and also perennial Cyperus species.

In the case of the dicotyledonous weed species, the spectrum of action extends to genera such as, for example, Galium, Viola, Veronica, Lamium, Stellaria, Amaranthus, Sinapis, Ipomoea, Matricaria, Abutilon and Sida, amongst the annuals, and Convolvulus, Cirsium, Rumex and Artemisia in the case of the perennial weeds. The active ingredients according to the invention also act outstandingly efficiently on harmful plants which are found under the specific cultures in rice, such as, for example, Sagittaria, Alisma, Eleocharis, Scirpus and Cyperus.

When the compounds according to the invention are applied to the earth surface before germination, the weed seedlings are either prevented completely from emerging or else the weeds grow until they have reached the cotyledon stage, but then their growth stops, and, eventually, after three to four weeks have elapsed, they die completely.

When the active ingredients are applied post-emergence to the green parts of the plants, growth likewise stops drastically a very short time after the treatment, and the weed plants remain at the growth stage of the point of time of application, or they die completely after a certain time, so that in this manner competition by the weeds, which is harmful to the crop plants, is eliminated very early and in a sustained manner.

Even though the compounds according to the invention have an outstanding herbicidal activity against undesired monocotyledonous and dicotyledonous plants, crop plants of economically important crops such as, for example, wheat, barley, rye, rice, maize, sugarbeet cotton and soybean, are harmed only to a minor extent, if at all. For these reasons, the present compounds are highly suitable for the selective control of undesired vegetation in stands of agriculturally useful plants or of ornamentals.

Moreover, the substances according to the invention have outstanding growth-regulatory properties in crop plants. They engage in the plants' metabolism in a regulatory fashion and can therefore be employed for the tailor-made control of plant constituents and for facilitating harvesting such as, for example, by triggering desiccation and stunted growth. Moreover, they are also suitable for generally controlling and inhibiting undesired vegetative growth without destroying the plants in the process. Inhibiting vegetative growth plays an important role in many monocotyledonous and dicotyledonous crops since lodging can be thereby reduced or prevented fully.

Owing to their herbicidal and plant-growth regulatory properties, the active ingredients can also be employed for controlling harmful plants in crops of genetically modified plants which are known or yet to be developed. As a rule, the transgenic plants are distinguished by particular advantageous properties, for example by resistances to certain pesticides, especially certain herbicides, resistances to plant diseases or to plant pathogens such as certain insects or microorganisms such as fungi, bacteria or viruses. Other particular properties concern for example the harvested material with regard to quantity, quality, storability, composition and specific constituents. Thus, transgenic plants with an increased starch content or with a modified starch quality or with a different fatty acid composition of the harvested material are known.

The use of compounds of the formula (I) according to the invention or their salts in economically important transgenic crops of useful plants and ornamentals, such as of cereals such as wheat, barley, rye, oats, sorghum and millet, rice, cassaya and maize or else crops of sugarbeet, cotton, soybean, oilseed rape, potato, tomato, pea and other vegetables is preferred.

The compounds of the formula (I) may preferably be employed as herbicides in crops of useful plants which are resistant to the phytotoxic effects of the herbicides or which have been rendered resistant to the phytotoxic effects of the herbicides by recombinant is.

Conventional methods of generating novel plants which have modified properties in comparison to plants occurring to date consists, for example, in traditional breeding methods and the generation of mutants. Alternatively, novel plants with modified properties can be generated with the aid of recombinant methods (see, for example, EP-A-0221044, EP-A-0131624). For example, the following have been described in several cases:

the modification, by recombinant technology, of crop plants with the aim of modifying the starch synthesized in the plants (for example WO 92/11376, WO 92/14827, WO 91/19806),

transgenic crop plants which are resistant to certain herbicides of the type glufosinate (cf., for example, EP-A-0242236, EP-A-242246) or glyphosate (WO 92/00377) or of the sulfonylureas (EP-A-0257993, U.S. Pat. No. 5,013,659),

transgenic crop plants, for example cotton, with the capability of producing Bacillus thuringiensis toxins (Bt toxins), which make the plants resistant to certain pests (EP-A-0142924, EP-A-0193259),

transgenic crop plants with a modified fatty acid composition (WO 91/13972).

A large number of molecular biological techniques are known in principle with the aid of which novel transgenic plants with modified properties can be generated; see, for example, Sambrook et al., 1989, Molecular Cloning, A Laboratory Manual, 2nd Ed., Cold Spring Harbor Laboratory Press, Cold Spring Harbor, N.Y.; or Winnacker “Gene und Klone” [Genes and Clones], VCH Weinheim 2nd Edition 1996, or Christou, “Trends in Plant Science” 1 (1996) 423-431).

To carry out such genetic manipulations, nucleic acid molecules which allow mutagenesis or sequence changes by recombination of DNA sequences can be introduced into plasmids. For example, the abovementioned standard methods allow base exchanges to be carried out, subsequences to be removed, or natural or synthetic sequences to be added. To connect the DNA fragments to each other, adapters or linkers may be added to the fragments.

For example, the generation of plant cells with a reduced activity of a gene product can be achieved by expressing at least one corresponding antisense RNA, a sense RNA for achieving a cosuppression effect or by expressing at least one suitably constructed ribozyme which specifically cleaves transcripts of the above-mentioned gene product.

To this end, it is possible to use, on the one hand, DNA molecules which encompass the entire coding sequence of a gene product inclusive of any flanking sequences which may be present, as well as DNA molecules which only encompass portions of the coding sequence, it being necessary for these portions to be long enough to have an antisense effect on the cells. The use of DNA sequences which have a high degree of homology to the coding sequences of a gene product, but which are not completely identical to them, is also possible.

When expressing nucleic acid molecules in plants, the protein synthesized can be localized in any desired compartment of the plant cell. However, to achieve localization in a particular compartment, it is possible, for example, to link the coding region with DNA sequences which ensure localization in a particular compartment. Such sequences are known to those skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106).

The transgenic plant cells can be regenerated by known techniques to give rise to intact plants. In principle, the transgenic plants can be plants of any desired species, i.e. both monocots and dicots.

Thus, transgenic plants can be obtained whose properties are altered by overexpression, suppression or inhibition of homologous (=natural) genes or gene sequences or the expression of heterologous (=foreign) genes or gene sequences.

The compounds (I) according to the invention can preferably be employed in transgenic crops which are resistant to herbicides from the group of the sulfonylureas, glufosinate-ammonium or glyphosate-isopropylammonium and analogous active ingredients.

When the active ingredients according to the invention are used in transgenic crops, effects are frequently observed, in addition to the effects against harmful plants observed in other crops, which are specific for the application in the transgenic crop in question, for example a modified or specifically extended weed spectrum which can be controlled, altered application rates which can be employed for application, preferably good combining properties with the herbicides to which the transgenic crop is resistant, and an effect on growth and yield of the transgenic crop plants.

The invention therefore also relates to the use of the compounds (I) according to the invention as herbicides for controlling harmful plants in transgenic crop plants.

The use according to the invention for controlling harmful plants or for regulating the growth of plants also includes the case where the active ingredient of the formula (I) or its salt is only formed post-application on the plant, in the plant or in the soil starting from a prodrug.

The compounds according to the invention can be applied in the form of wettable powders, emulsifiable concentrates, sprayable solutions, dusts or granules in the customary preparations. The invention therefore also relates to herbicidal and plantgrowth regulatory compositions which comprise compounds of the formula (I).

The compounds of the formula (I) can be formulated in many ways, depending on the biological and/or chemicophysical parameters which prevail. The following are examples of possibilities for formulations: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), oil- or water-based dispersions, oil-miscible solutions, capsule suspensions (CS), dusts (DP), seed-dressing materials, granules for broadcasting and soil application, granules (GR) in the form of microgranules, spray granules, coated granules and adsorption granules, water-dispersible granules (WG), water-soluble granules (SG), ULV formulations, microcapsules and waxes. These individual formulation types are known in principle and are described, for example, in Winnacker-Küchler, “Chemische Technologie” [Chemical Engineering], Volume 7, C. HauserVerlag Munich, 4th Ed. 1986, Wade van Valkenburg, “Pesticide Formulations”, Marcel Dekker, N.Y., 1973; K. Martens, “Spray Drying” Handbook, 3rd Ed. 1979, G. Goodwin Ltd. London.

The formulation auxiliaries required, such as inert materials, surfactants, solvents and other additives, are also known and are described, for example, in Watkins, “Handbook of Insecticide Dust Diluents and Carriers”, 2nd Ed., Darland Books, Caldwell N.J., H. v. Olphen, “Introduction to Clay Colloid Chemistry”; 2nd Ed., J. Wiley & Sons, N.Y.; C. Marsden, “Solvents Guide”; 2nd Ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. PubI. Co. Inc., N.Y. 1964; Schönfeldt, “Grenzflächenaktive Äthylenoxidaddukte”, [Surface-active ethylene oxide adducts], Wiss. Verlagsgesell., Stuttgart 1976; Winnacker-Küchler, “Chemische Technologie”, Volume 7, C. HauserVerlag Munich, 4th Edition 1986.

Wettable powders are products which are uniformly dispersible in water and which, besides the active ingredient, also comprise ionic and/or nonionic surfactants (wetters, dispersants), for example polyoxyethylated alkylphenols, polyethoxylated fatty alcohols, polyethoxylated fatty amines, fatty alcohol polyglycol ether sulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltauride, in addition to a diluent or inert material. To prepare the wettable powders, the herbicidal active ingredients are ground finely, for example in customary apparatuses such as hammer mills, blower mills and air-jet mills, and mixed with the formulation auxiliaries, either simultaneously or subsequently.

Emulsifiable concentrates are prepared by dissolving the active ingredient in an organic solvent, for example butanol, cyclohexanone, dimethylformamide, xylene or else higher-boiling aromatics or hydrocarbons or mixtures of the organic solvents with addition of one or more ionic and/or nonionic surfactants (emulsifiers). Examples of emulsifiers which may be used are: calcium salts of alkylarylsulfonic acids, such as calcium dodecylbenzenesulfonate, or nonionic emulsifiers such as fatty acid polyglycol esters, alkylaryl polyglycol ethers, fatty alcohol polyglycol ethers, propylene oxide/ethylene oxide condensates, alkyl polyethers, sorbitan esters such as sorbitan fatty acid esters, for example, or polyoxyethylene sorbitan esters such as, for example, polyoxyethylene sorbitan fatty acid esters.

Dusts are obtained by grinding the active ingredient with finely divided solid materials, for example talc, natural clays such as kaolin, bentonite and pyrophyllite, or diatomaceous earth.

Suspension concentrates can be water- or oil-based. They can be prepared, for example, by wet grinding by is of commercially available bead mills and, if appropriate, addition of surfactants as have already been mentioned for example above in the case of the other formulation types.

Emulsions, for example oil-in-water emulsions (EW), can be prepared for example by is of stirrers, colloid mills and/or static mixers using aqueous organic solvents and, if appropriate, surfactants as have already been mentioned for example above in the case of the other formulation types.

Granules can be prepared either by spraying the active ingredient onto adsorptive, granulated inert material or by applying active ingredient concentrates to the surface of carriers such as sand, kaolinites or granulated inert material with the aid of adhesives, for example polyvinylalcohol, sodium polyacrylate or else mineral oils. Suitable active ingredients may also be granulated in the manner conventionally used for the production of fertilizer granules, if desired in a mixture with fertilizers.

As a rule, water-dispersible granules are prepared by conventional processes such as spray drying, fluidized-bed granulation, disk granulation, mixing with high-speed mixers and extrusion without solid inert material.

Regarding the production of disk granules, fluidized-bed granules, extruder granules and spray granules, see, for example, the methods in “Spray-Drying Handbook” 3rd Ed. 1979, G. Goodwin Ltd., London; J. E. Browning, “Agglomeration”, Chemical and Engineering 1967, page 147 et seq; “Perry's Chemical Engineer's Handbook”, 5th Ed., McGraw-Hill, New York 1973, pp. 8-57.

For further details on the formulation of crop protection products, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

As a rule, the agrochemical formulations comprise 0.1 to 99% by weight, in particular 0.1 to 95% by weight, of active ingredient of the formula (I).

The active ingredient concentration in wettable powders is, for example, approximately 10 to 90% by weight, the remainder to 100% by weight being composed of customary formulation constituents. In the case of emulsifiable concentrates, the active ingredient concentration may amount to approximately 1 to 90%, preferably 5 to 80% by weight. Formulations in the form of dusts comprise 1 to 30% by weight of active ingredient, preferably in most cases 5 to 20% by weight of active ingredient, sprayable solutions contain approximately 0.05 to 80%, preferably 2 to 50% by weight of active ingredient. In the case of water dispersible granules, the active ingredient content depends partly on whether the active compound is present in liquid or solid form and on which granulation auxiliaries, fillers and the like are being used. The active ingredient content amounts to, for example, between 1 and 95% by weight, preferably to between 10 and 80% by weight in the case of the water-dispersible granules.

In addition, the abovementioned active ingredient formulations may comprise, if appropriate, the adhesives, wetters, dispersants, emulsifiers, penetrants, preservatives, antifreeze agents, solvents, fillers, carriers, colorants, antifoams, evaporation inhibitors, pH regulators and viscosity regulators which are customary in each case.

The compounds of the formula (I) or their salts can be employed as such or in the form of their preparations (formulations) together with other pesticidally active substances, such as, for example, insecticides, acaricides, nematicides, herbicides, fungicides, safeners, fertilizers and/or growth regulators, for example as a readymix or as tank mixes. The combination formulations can be prepared in this case on the basis of the abovementioned formulations, taking into consideration the physical properties and stabilities of the active substances to be combined.

Components in combinations for the active ingredients according to the invention in formulation mixtures or in the tank mix are, for example, known active ingredients based on the inhibition of, for example, acetolactate synthase, acetyl-coenzyme A carboxylase, PS I, PS II, HPPDO, phytoene desaturase, protoporphyrinogen oxidase, glutamine synthetase, cellulose biosynthesis, or 5-enolpyruvylshikimate-3-phosphate synthetase. Such compounds and also other compounds which can be employed and which have a mechanism of action which is either partially unknown or different are described, for example, in Weed Research 26, 441-445 (1986) or “The Pesticide Manual”, 12th edition 2000, edited by The British Crop Protection Council (abbreviated to “PM” hereinbelow), and the literature cited therein. Examples of herbicides known from the literature which can be combined with the compounds of the formula (I) are the following active ingredients (note: either the common name in accordance with the International Organization for Standardization (ISO) or the chemical name of the compounds, if appropriate together with the customary code number, is given):

acetochlor; acifluorfen(-sodium); aclonifen; AKH 7088, i.e. [[[1-[5-[2-chloro4-(trifluoromethyl)phenoxy]-2-nitrophenyl]-2-methoxyethylidene]amino]oxy]acetic acid and its methyl ester; alachlor; alloxydim(-sodium); ametryn; amicarbazone; amidochlor, amidosulfuron; amitrol; AMS, i.e. ammonium sulfamate; anilofos; asulam; atrazine; azalfenidin; azimsulfurone (DPX-A8947); aziprotryn; barban; BAS 516H, i.e. 5-fluoro-2-phenyl-4H-3,1-benzoxazin-4-one; beflubutamid; benazolin(-ethyl); benfluralin; benfuresate; bensulfuron(-methyl); bensulide; bentazone; benzobicyclon; benzofenap; benzofluor; benzoylprop(-ethyl); benzthiazuron; bialaphos; bifenox; bispyribac(-sodium) bromacil; bromobutide; bromofenoxim; bromoxynil; bromuron; buminafos; busoxinone; butachlor; butafenacil; butamifos; butenachlor; buthidazole; butralin; butroxydim; butylate; cafenstrole (CH-900); carbetamide; cafentrazone(-ethyl)(IC1—A0051); caloxydim; CDM, i.e. 2-chloro-N,N-di-2-propenylacetamide; CDEC, i.e. 2-chloroallyl diethyl dithiocarbamate; chlomethoxyfen; chloramben; chlorazifop-butyl, chlormesulon (ICI-A0051); chlorbromuron; chlorbufam; chlorfenac; chlorflurecol-methyl; chloridazon; chlorimuron(-ethyl); chlornitrofen; chlorotoluron; chloroxuron; chlorpropham; chlorsulfuron; chlorthal-dimethyl; chlorthiamid; chlortoluron; cinidon(-methyl and -ethyl), cinmethylin; cinosulfuron; clefoxydim; clethodim; clodinafop and its ester derivatives (for example clodinafop-propargyl); clomazone; clomeprop; cloproxydim; clopyralid; clopyrasulfuron(-methyl), cloransulam(-methyl), cumyluron (JC 940); cyanazine; cycloate; cyclosulfamuron (AC 104); cycloxydim; cycluron; cyhalofop and its ester derivatives (for example butyl ester, DEH-112); cyperquat; cyprazine; cyprazole; daimuron; 2,4-D; 2,4-DB; dalapon; desmedipham; desmetryn; di-allate; dicamba; dichlobenil; dichlorprop; diclofop and its esters such as diclofop-methyl; diclosulam, diethatyl(-ethyl); difenoxuron; difenzoquat; diflufenican; diflufenzopyr; dimefuron; dimepiperate, dimethachlor; dimethametryn; dimethenamid (SAN-582H); dimethazone,dimexyflam; dimethipin; dimetrasulfuron, dinitramine; dinoseb; dinoterb; diphenamid; dipropetryn; diquat; dithiopyr; diuron; DNOC; eglinazine-ethyl; EL 77, i.e. 5-cyano-1-(1,1-dimethylethyl)-N-methyl-1H-pyrazole-4-carboxamide; endothal; epoprodan; EPTC; esprocarb; ethalfluralin; ethametsulfuron-methyl; ethidimuron; ethiozin; ethofumesate; ethoxyfen and its esters (for example ethyl ester, HN-252); ethoxysulfuron, etobenzanid (HW 52); F5231, i.e. N-[2-chloro-4-fluoro-5[4-(3-fluoropropyl)-4,5-dihydro-5-oxo-1H-tetrazol-1-yl]phenyl]ethanesulfonamide; fenoprop; fenoxan, fenoxaprop and fenoxaprop-P and their esters, for example fenoxaprop-P-ethyl and fenoxaprop-ethyl; fenoxydim; fentrazamide, fenuron; flamprop(-methyl or -isopropyl or -isopropyl-4); flazasulfuron; floazulate, florasulam, fluazifop and fluazifop-P and their esters, for example fluazifop-butyl and fluazifop-P-butyl; flucarbazone(-sodium), fluchloralin; flumetsulam; flumeturon; flumiclorac(-pentyl), flumioxazin (S-482); flumipropyn; flumeturon, fluorochloridone, fluorodifen, fluoroglycofen(-ethyl); flupoxam (KNW-739); flupropacil (UBIC-4243); flupyrsulfuron(-methyl or -sodium), flurenol(-butyl), fluridone; flurochloridone; fluroxypyr(-meptyl) flurprimidol; flurtamone; fluthiacet(-methyl), fluthiamide, fomesafen; foramsulfuron, fosamine; furyloxyfen; glufosinate(-ammonium); glyphosate(-isopropylammonium); halosafen; halosulfuron(-methyl) and its esters (for example methylester, NC-319); haloxyfop and its esters; haloxyfop-P (═R-haloxyfop) and its esters; hexazinone; imazamethabenz(-methyl); imazapyr; imazaquin and salts such as the ammonium salt; imazamethapyr, imazamox, imazapic, imazethamethapyr; imazethapyr; imazosulfuron; indanofan, ioxynil; isocarbamid; isopropalin; isoproturon; isouron; isoxaben; isoxachlortole, isoxaflutole, isoxapyrifop; karbutilate; lactofen; lenacil; linuron; MCPA; MCPB; mecoprop; mefenacet; mefluidid; mesosulfuron, mesotrione, metamitron; metazachlor; methabenzthiazuron; metham; methazole; methoxyphenone; methyldymron; metabenzuron, methobenzuron; metobromuron; (alpha-)metolachlor; metosulam (XRD 511); metoxuron; metribuzin; metsulfuron-methyl; MH; molinate; monalide; monocarbamide dihydrogensulfate; monolinuron; monuron; MT 128, i.e. 6-chloro-N-(3-chloro-2-propenyl)-5-methyl-N-phenyl-3-pyridazinamine; MT 5950, i.e. N-[3-chloro-4-(1-methyl-ethyl)phenyl]-2-methylpentanamide; naproanilide; napropamide; naptalam; NC 310, i.e. 4-(2,4-dichlorobenzoyl)-1-methyl-5-benzyloxypyrazol; neburon; nicosulfuron; nipyraclophen; nitralin; nitrofen; nitrofluorfen; norfiurazon; orbencarb; oryzalin; oxadiargyl (RP-020630); oxadiazon; oxasulfuron, oxaziclomefone, oxyfluorfen; paraquat; pebulate; pelargonic acid, pendimethalin; pentoxazone, perfluidone; phenisopham; phenmedipham; picloram; picolinafen, piperophos; piributicarb; pirifenop-butyl; pretilachlor; primisulfuron(-methyl); procarbazone-(sodium), procyazine; prodiamine; profluralin; proglinazine(-ethyl); prometon; prometryn; propachlor; propanil; propaquizafop and its esters; propazine; propham; propisochlor; propyzamide; prosulfalin; prosulfocarb; prosulfuron (CGA-152005); prynachlor; pyraflufen(-ethyl), pyrazolinate; pyrazon; pyrazosulfuron(-ethyl); pyrazoxyfen; pyribenzoxim, pyributicarb, pyridafol, pyridate; pyrimidobac(-methyl), pyrithiobac(-sodium) (K1H-2031); pyroxofop and its esters (for example propargyl ester); quinclorac; quinmerac; quinoclamine, quinofop and its ester derivatives, quizalofop and quizalofop-P and their ester derivatives, for example quizalofop-ethyl; quizalofop-P-tefuryl and -ethyl; renriduron; rimsulfuron (DPX-E 9636); S 275, i.e. 2-[4-chloro-2-fluoro-5-(2-propynyloxy)phenyl]-4,5,6,7-tetrahydro-2H-indazole; secbumeton; sethoxydim; siduron; simazine; simetryn; SN 106279, i.e. 2-[[7-[2-chloro-4-(trifluoromethyl)phenoxy]-2-naphthalenyl]oxy]propanoic acid and methyl ester; sulcotrione, sulfentrazon (FMC-97285, F-6285); sulfazuron; sulfometuron(-methyl); sulfosate (IC1—A0224); sulfosulfuron, TCA; tebutam (GCP-5544); tebuthiuron; tepraloxydim, terbacil; terbucarb; terbuchlor; terbumeton; terbuthylazine; terbutryn; TFH 450, i.e. N,N-diethyl-3-[(2-ethyl-6-methyl-phenyl)sulfonyl]-1H-1,2,4-triazol-1-carboxamide; thenylchlor (NSK-850); thiafluamide, thiazafluron; thiazopyr (Mon-13200); thidiazimin (SN-24085); thifensulfuron(-methyl); thiobencarb; tiocarbazil; tralkoxydim; tri-allate; triasulfuron; triaziflam, triazofenamide; tribenuron(-methyl); triclopyr; tridiphane; trietazine; trifluralin; triflusulfuron and esters (for example methyl ester, DPX-66037); trimeturon; tritosulfuron, tsitodef; vernolate; WL 110547, i.e. 5-phenoxy-1-[3-(trifluoromethyl)phenyl]-1H-tetrazole; BAY MKH 6561, UBH-509; D-489; LS 82-556; KPP-300; NC-324; NC-330; KH-218; DPX—N8189; SC-0774; DOWCO-535; DK-8910; V-53482; PP-600; MBH-001; KIH-9201; ET-751; KIH-6127 and KIH-2023.

For use, the formulations which are present in commercially available form are, if appropriate, diluted in the customary manner, for example using water in the case of wettable powders, emulsifiable concentrates, dispersions and water-dispersible granules. Preparations in the form of dusts, soil granules, granules for spreading, and sprayable solutions are usually not diluted any further with other inert substances prior to use.

The required application rate of the compounds of the formula (I) varies with the external conditions such as, inter alia, temperature, humidity and the nature of the herbicide used. It can vary within wide limits, for example between 0.001 and 10.0 kg/ha or more of active substance, but it is preferably between 0.005 and 5 kg/ha.

In the examples which follow, amounts (also percentages) refer to weight, unless specifically indicated otherwise.

### A. CHEMICAL EXAMPLES

### Example A1

2-Amino-4-(1-fluoro-1-methylethyl)-6-[1-(trans-2-phenylcyclopropyl)prop-1-ylamino]-1,3,5-triazine (see Table 2, Example 2-1)

A solution prepared with 0.32 g (0.014 mol) of sodium and 10 ml of methanol is added to 1.81 g (0.0061 mol) of 1-[1-(trans-2-phenylcyclopropyl)prop-1-yl]-biguamide hydrochloride in 30 ml of methanol and 2 g of molecular sieve 3 A (Ångström). Thereafter, 1.51 g (0.0126 mol) of methyl 2-fluoro-2-methylpropionate are added dropwise and the mixture is first stirred for 2 hours at 25° C. and then for 4 hours at 65° C. The reaction mixture is filtered, the filtrate is concentrated, and the residue is taken up in ethyl acetate. The mixture is washed with water and dried with sodium sulfate. The dessicant is filtered off with suction, and the solvent is evaporated in vacuo. Purification by column chromatography (eluent: ethyl acetate) gives 1.60 g (83% of theory) of 2-amino-4-(1-fluoro-1-methylethyl)-6-[11-(trans-2-phenylcyclopropyl)-1-propylamino]-1,3,5-triazine.

### Example A2

2-Amino-4-(1-fluoroethyl)-6-[1-(trans-2-phenylcyclopropyl)-1-ethylamino]-1,3,5-triazine (see Table 1, Example 1-2)

A solution of 1.29 g (0.008 mol) of 1-(trans-2-phenylcyclopropyl)ethylamine), dissolved in 10 ml of acetonitrile, is added to a solution of 1.41 g (0.008 mol) of 2-amino-6-chloro-4-(1-fluorethyl)-1,3,5-triazine and 1.64 g (0.012 mol) of potassium carbonate in 30 ml of acetonitrile. The mixture is refluxed for three hours. The solid constituents are then filtered off with suction and the filtrate is evaporated on a rotary evaporator. The residue is purified by column chromatography (eluent: methyl acetate). This gives 2.34 g (93% of theory) of 2-amino-4-(1-fluoroethyl)-6-[11-(trans-2-phenylcyclopropyl)-1-ethylamino]-1,3,5-triazine.

The compounds described in Tables 1 to 7 hereinbelow are obtained in accordance with, or analogously to, the above Examples A1 and A2 or the methods described further above in general terms. The following abbreviations are used in the tables:

| | |
|---|---|
| Me = | methyl |
| Et = | ethyl |
| Pr = | propyl |
| i-Pr = | isopropyl |
| c-Pr = | cyclopropyl or, in the case of two free bonds, cyclopropylene |
| Bu = | n-butyl |
| sec-Bu = | secondary butyl, 2-butyl |
| c-Bu = | cyclobutyl or, in the case of two free bonds, cyclobutylene |
| c-Pen = | cyclopentyl or, in the case of two free bonds, cyclopentylene |
| c-Hex = | cyclohexyl or, in the case of two free bonds, cyclohexylene |
| THF = | tetrahydrofuryl or, in the case of two free bonds, tetrahydro- |
| | furylene (oxolanylene, oxolanediyl) |

| | | |
|---|---|---|
| trans-1,2-c-Pr- = | trans-1,2-cyclopropylene = | 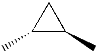 |

(the stereochemically characterized bonds in the formula and in the
formulae which follow are free bonds and do not mean the abbreviations
of methyl or methylene group)

| | | |
|---|---|---|
| cis-1,2-c-Pr- = | cis-1,2-cyclopropylene = | 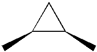 |

(analogous abbreviations apply to cis-1,2-c-Bu and the like)

| | | |
|---|---|---|
| trans-1,2-Ox- = | trans-1,2-oxiranylene = | 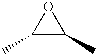 |
| cis-1,2-Ox- = | cis-1,2-oxiranylene = | 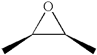 |
| cis-1,2-Az(Me)- = | N-methyl-cis-1,2-aziridinylene = | |
| trans-1,2-Az(Me)- = | N-methyl-trans-1,2-aziridinylene = | 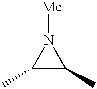 |

| | | |
|---|---|---|
| A1 = | (CH₂)₁= | —CH₂— |
| A2 = | —(CH₂)₂— = | —CH₂CH₂— |
| A3 = | —(CH₂)₃— = | —CH₂CH₂CH₂— |
| A4 = | —(CH₂)₄— = | —CH₂CH₂CH₂CH₂— |
| Ac = | COCH₃ = | acetyl |
| Ph = | phenyl | |
| (X)ₙ = | “-”is n = 0 | |

Tables 1 to 7 which follow refer to formula (1a)

TABLE 1
Compounds of the formula (1a)

| | | | | | | (1a) |
|---|---|---|---|---|---|---|
| 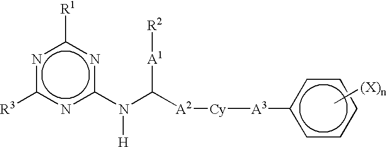 | | | | | | |
| | | | | | | Phys. |
| No. | R¹ | —A¹—R² | R³ | A²—Cy—A³ | (X)ₙ | Data |
| 1-1 | CFMe₂ | Me | NH₂ | trans-1,2-c-Pr- | — | Oil |
| 1-2 | CHFMe | Me | NH₂ | trans-1,2-c-Pr- | — | Oil |
| 1-3 | CHFEt | Me | NH₂ | trans-1,2-c-Pr- | — | Oil |
| 1-4 | i-Pr | Me | NH₂ | trans-1,2-c-Pr- | — | Oil |
| 1-5 | Me | Me | NH₂ | trans-1,2-c-Pr- | — | |
| 1-6 | Et | Me | NH₂ | trans-1,2-c-Pr- | — | Oil |
| 1-7 | CFMe₂ | Me | NH₂ | trans-1,2-c-Pr- | 3-Cl | |
| 1-8 | CHFMe | Me | NH₂ | trans-1,2-c-Pr- | 3-Cl | |
| 1-9 | CHFEt | Me | NH₂ | trans-1,2-c-Pr- | 3-Cl | |
| 1-10 | CF₃ | Me | NH₂ | trans-1,2-c-Pr- | 3-Cl | |
| 1-11 | CFMe₂ | Me | NH₂ | trans-1,2-c-Pr- | 3-F | |
| 1-12 | CHFMe | Me | NH₂ | trans-1,2-c-Pr- | 3-F | |
| 1-13 | CHFEt | Me | NH₂ | trans-1,2-c-Pr- | 3-F | |
| 1-14 | CHFPr | Me | NH₂ | trans-1,2-c-Pr- | 3-F | |
| 1-15 | CFMe₂ | Me | NH₂ | trans-1,2-c-Pr- | 3-OMe | |
| 1-16 | i-Pr | Me | NH₂ | trans-1,2-c-Pr- | 3-OMe | |
| 1-17 | CFMe₂ | Me | NHCOMe | trans-1,2-c-Pr- | — | |
| 1-18 | CFMe₂ | Me | NHCOEt | trans-1,2-c-Pr- | — | |
| 1-19 | CFMe₂ | Me | N═CHNMe₂ | trans-1,2-c-Pr- | — | |
| 1-20 | CFMe₂ | Me | N═C(Me)NMe₂ | trans-1,2-c-Pr- | — | |
| 1-21 | CFMe₂ | Me | N═CHNEt₂ | trans-1,2-c-Pr- | — | |
| 1-22 | CFMe₂ | Me | NHCONHEt | trans-1,2-c-Pr- | — | |
| 1-23 | CFMe₂ | Me | NH₂ | trans-1,2-c-Pr- | — | |
| 1-24 | CFMe₂ | Me | NH₂ | cis-1,2-c-Pr- | — | |
| 1-25 | CClMe₂ | Me | NH₂ | cis-1,2-c-Pr- | 2,3-Cl₂ | |
| 1-26 | CBrMe₂ | Me | NH₂ | cis-1,2-c-Pr- | 4-OMe | |
| 1-27 | CFEt₂ | Me | NH₂ | cis-1,2-c-Pr- | 3-OMe | |
| 1-28 | Ph | Me | NH₂ | cis-1,2-c-Pr- | 2-OMe | |
| 1-29 | 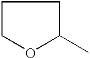 | Me | NH₂ | cis-1,2-c-Pr- | — | |
| 1-30 | 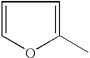 | Me | NH₂ | cis-1,2-c-Pr- | — | |
| 1-31 | 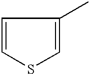 | Me | NH₂ | cis-1,2-c-Pr- | — | |
| 1-32 | 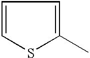 | Me | NH₂ | cis-1,2-c-Pr- | — | |
| 1-33 | 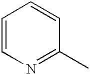 | Me | NH₂ | cis-1,2-c-Pr- | 3-CF₃ | |

TABLE 2
Compounds of the formula (1a)

| | | | | | | Phys. |
|---|---|---|---|---|---|---|
| No. | R¹ | -A¹-R² | R³ | A²-Cy-A³ | (X)ₙ | Data |
| 2-1 | CFMe₂ | Et | NH₂ | trans-1,2- | — | Oil |
| | | | | c-Pr | | |
| 2-2 | i-Pr | Et | NH₂ | trans-1,2- | — | Oil |
| | | | | c-Pr | | |
| 2-3 | CHFMe | Et | NH₂ | trans-1,2- | — | Oil |
| | | | | c-Pr | | |
| 2-4 | CF₃ | Et | NH₂ | trans-1,2- | — | |
| | | | | c-Pr | | |
| 2-5 | CF₂ CHF₂ | Et | NH₂ | trans-1,2- | — | |
| | | | | c-Pr | | |
| 2-6 | CHF₂ | Et | NH₂ | trans-1,2- | — | |
| | | | | c-Pr | | |
| 2-7 | CHFMe | Et | NH₂ | trans-1,2- | — | |
| | | | | c-Pr | | |
| 2-8 | CHFEt | Et | NH₂ | trans-1,2- | — | Oil |
| | | | | c-Pr | | |
| 2-9 | CFMeEt | Et | NH₂ | trans-1,2- | 3,5-Cl₂ | |
| | | | | c-Pr | | |
| 2-10 | CFMe₂ | Et | NH₂ | trans-1,2- | 3,5-Cl₂ | |
| | | | | c-Pr | | |
| 2-11 | CHFPr | Et | NH₂ | trans-1,2- | 3,5-Cl₂ | |
| | | | | c-Pr | | |
| 2-12 | CHFEt | Et | NH₂ | trans-1,2- | 3,5-Cl₂ | |
| | | | | c-Pr | | |
| 2-13 | CFMe₂ | Et | NH₂ | trans-1,2- | 3-Me | |
| | | | | c-Pr | | |
| 2-14 | CFEt₂ | Et | NH₂ | trans-1,2- | 3-Me | |
| | | | | c-Pr | | |
| 2-15 | CH₂F | Et | NH₂ | trans-1,2- | 3-Me | |
| 2-16 | CHFEt | Et | NHCOCH₂Cl | trans-1,2- | 3-Cl | |
| | | | | c-Pr | | |
| 2-17 | CHFMe | Et | NHCOEt | trans-1,2- | 3-I | |
| | | | | c-Pr | | |

TABLE 3
Compounds of the formula (1a)

| | | | | | | Phys. |
|---|---|---|---|---|---|---|
| No. | R¹ | -A¹-R² | R³ | A²-Cy-A³ | (X)ₙ | Data |
| 3-1 | CFMe₂ | c-Pr | NH₂ | trans-1,2-c-Pr | — | Oil |
| 3-2 | i-Pr | c-Pr | NH₂ | trans-1,2-c-Pr- | — | Oil |
| 3-3 | CHFMe | c-Pr | NH₂ | trans-1,2-c-Pr- | — | Oil |
| 3-4 | CHFEt | c-Pr | NH₂ | trans-1,2-c-Pr- | — | Oil |
| 3-5 | CHFPr | c-Pr | NH₂ | trans-1,2-c-Pr- | — | |
| 3-6 | CFMeEt | c-Pr | NH₂ | trans-1,2-c-Pr- | — | |
| 3-7 | c-Pr | c-Pr | NH₂ | trans-1,2-c-Pr- | — | |
| 3-8 | c-Bu | c-Pr | NH₂ | trans-1,2-c-Pr- | — | |
| 3-9 | Ph | c-Pr | NH₂ | trans-1,2-c-Pr- | — | |
| 3-10 | 1-F-c-Pr | c-Pr | NH₂ | trans-1,2-c-Pr- | — | |
| 3-11 | 1-F-c-Bu | c-Pr | NH₂ | trans-1,2-c-Pr- | — | |
| 3-12 | CF₂Cl | c-Pr | NH₂ | trans-1,2-c-Pr- | — | |
| 3-13 | CCl₃ | c-Pr | NH₂ | trans-1,2-c-Pr- | — | |
| 3-14 | CF₂Br | c-Pr | NH₂ | trans-1,2-c-Pr- | — | |
| 3-15 | 4-Cl-Ph | c-Pr | NH₂ | trans-1,2-c-Pr- | — | |
| 3-16 | CFMe₂ | c-Pr | NH₂ | trans-1,2-c-Pr- | 3-Cl | |
| 3-17 | CHFMe | c-Pr | NH₂ | trans-1,2-c-Pr- | 3-Cl | |
| 3-18 | i-Pr | c-Pr | NH₂ | trans-1,2-c-Pr- | 3-Cl | |
| 3-19 | CHFEt | c-Pr | NH₂ | trans-1,2-c-Pr- | 3-Cl | |
| 3-20 | CFMe₂ | c-Pr | NH₂ | trans-1,2-c-Pr- | 3-Me | |
| 3-21 | i-Pr | c-Pr | NH₂ | trans-1,2-c-Pr- | 3-Me | |
| 3-22 | CHFMe | c-Pr | NH₂ | trans-1,2-c-Pr- | 3-Me | |
| 3-23 | CFMe₂ | c-Pr | NH₂ | trans-1,2-c-Pr- | 3,5-Me₂ | |
| 3-24 | CHFEt | c-Pr | NH₂ | trans-1,2-c-Pr- | 3,5-Me₂ | |
| 3-25 | CFMeEt | c-Pr | NH₂ | trans-1,2-c-Pr- | 3,5-Me₂ | |
| 3-26 | CHFMe | c-Pr | NH₂ | trans-1,2-c-Pr- | 3,5-Cl₂ | Oil |
| 3-27 | CHFEt | c-Pr | NH₂ | trans-1,2-c-Pr- | 3,5-Cl₂ | Oil |
| 3-28 | i-Pr | c-Pr | NH₂ | trans-1,2-c-Pr- | 3,5- Cl₂ | Oil |

TABLE 4
Compounds of the formula (1a)

| | | | | | | Phys. |
|---|---|---|---|---|---|---|
| No. | R¹ | —A¹—R² | R³ | A²—Cy—A³ | (X)ₙ | Data |
| 4-1 | CFMe₂ | c-Bu | NH₂ | trans-1,2-c-Pr- | — | Oil |
| 4-2 | CFEt₂ | c-Bu | NH₂ | trans-1,2-c-Pr- | — | |
| 4-3 | CHFMe | c-Bu | NH₂ | trans-1,2-c-Pr- | — | Oil |
| 4-4 | CHFEt | c-Bu | NH₂ | trans-1,2-c-Pr- | — | Oil |
| 4-5 | Me | c-Bu | NH₂ | trans-1,2-c-Pr- | — | |
| 4-6 | Et | c-Bu | NH₂ | trans-1,2-c-Pr- | — | |
| 4-7 | Ph | c-Bu | NH₂ | trans-1,2-c-Pr- | — | |
| 4-8 | 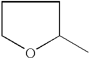 | c-Bu | NH₂ | trans-1,2-c-Pr- | — | |
| 4-9 | 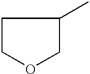 | c-Bu | NH₂ | trans-1,2-c-Pr- | — | |
| 4-10 | c-Pr | c-Bu | NH₂ | trans-1,2-c-Pr- | — | |
| 4-11 | c-Bu | c-Bu | NH₂ | trans-1,2-c-Pr- | — | |
| 4-12 | c-Pen | c-Bu | NH₂ | trans-1,2-c-Pr- | — | |
| 4-13 | CFMe₂ | c-Bu | NH₂ | trans-1,2-c-Pr- | 3-F | |
| 4-14 | CFMe₂ | c-Bu | NH₂ | trans-1,2-c-Pr- | 3,5-F₂ | |
| 4-15 | CFMe₂ | c-Bu | NH₂ | trans-1,2-c-Pr- | 3-I | |
| 4-16 | CFMe₂ | c-Bu | NH₂ | trans-1,2-c-Pr- | 4-SO₂Me | |
| 4-17 | CFMe₂ | c-Bu | NH₂ | trans-1,2-c-Pr- | 3-CF₃ | |
| 4-18 | CFMe₂ | c-Bu | NH—Ac | trans-1,2-c-Pr- | — | |
| 4-19 | CFEt₂ | c-Bu | NH—COPh | trans-1,2-c-Pr- | — | |
| 4-20 | CHFMe | c-Bu | NHCOCH₂Ph | trans-1,2-c-Pr- | — | |
| 4-21 | CHFMe | c-Bu | NEt₂ | trans-1,2-c-Pr- | — | |
| 4-22 | CHFMe | c-Bu | NHEt | trans-1,2-c-Pr- | — | |
| 4-23 | CHFMe | c-Bu | NHBu | trans-1,2-c-Pr- | — | |
| 4-24 | CHFMe | c-Bu | NHCH₂Ph | trans-1,2-c-Pr- | — | |
| 4-25 | CHFMe | c-Bu | N(Me)CH₂Ph | trans-1,2-c-Pr- | — | |
| 4-26 | CHFMe | c-Bu | N═CH—NMe₂ | trans-1,2-c-Pr- | — | |
| 4-27 | i-Pr | c-Bu | NH₂ | trans-1,2-c-Pr- | — | Oil |
| 4-28 | CFMe₂ | c-Bu | NH₂ | trans-1,2-c-Pr- | 3-Cl | Oil |

TABLE 5
Compounds of the formula (1a)

| | | | | | | Phys. |
|---|---|---|---|---|---|---|
| No. | R¹ | —A¹—R² | R³ | A²—Cy—A³ | (X)₂ | Data |
| 5-1 | CFMe₂ | A1-c-Pr | NH₂ | trans-1,2-c-Pr- | — | Oil |
| 5-2 | i-Pr | A1-c-Pr | NH₂ | trans-1,2-c-Pr- | — | Oil |
| 5-3 | CFEt₂ | A1-c-Pr | NH₂ | trans-1,2-c-Pr- | — | Oil |
| 5-4 | CHFEt | A1-c-Pr | NHCHO | trans-1,2-c-Pr- | — | Oil |
| 5-5 | CHF-i-Pr | A1-c-Pr | NHCHO | trans-1,2-c-Pr- | — | |
| 5-6 | CF₃ | A1-c-Pr | NHCHO | trans-1,2-c-Pr- | — | |
| 5-7 | CFEt₂ | A1-c-Pr | NHCOCH₂Cl | trans-1,2-c-Pr- | — | |
| 5-8 | CFMeEt | 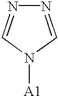 | NH₂ | trans-1,2-c-Pr- | — | |
| 5-9 | CF₂CF₃ | 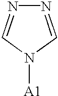 | NH₂ | trans-1,2-c-Pr- | — | |
| 5-10 | CFMe₂ | 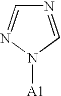 | NH₂ | trans-1,2-c-Pr- | — | |
| 5-11 | CFMe₂ | 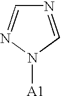 | NHCO-i-Pr | trans-1,2-c-Pr- | — | |
| 5-12 | CHFEt | 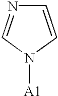 | NH₂ | trans-1,2-c-Pr- | 3,5- (OMe)₂ | |
| 5-13 | CHFPr | 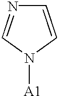 | NH₂ | trans-1,2-c-Pr- | — | |
| 5-14 | CFMe₂ | A2-c-Pr | NH₂ | trans-1,2-c-Pr- | — | |
| 5-15 | CF₃ | A2-c-Pr | NH₂ | trans-1,2-c-Pr- | — | |
| 5-16 | CClMe₂ | A2-c-Bu | NH₂ | trans-1,2-c-Pr- | — | |
| 5-17 | CFMe₂ | A2-c-Bu | NH₂ | trans-1,2-c-Pr- | — | |
| 5-18 | 4-Cl-Ph | Pr | NH₂ | trans-1,2-c-Pr- | — | |
| 5-19 | 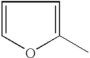 | i-Pr | N═CHNMe₂ | trans-1,2-c-Pr- | 2,3- (OMe)₂ | |
| 5-20 | 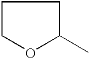 | i-Bu | 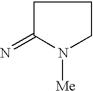 | trans-1,2-c-Pr- | 2,3- (OEt)₂ | |
| 5-21 | 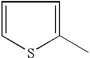 | Bu | N═CEt—NMe₂ | trans-1,2-c-Pr- | 2,3-F₂ | |
| 5-22 | CCl₃ | CF₃ | NH₂ | trans-1,2-c-Pr- | 3-Et | |
| 5-23 | CH₂Cl | CFMe₂ | NH₂ | trans-1,2-c-Pr- | 3-Pr | |
| 5-24 | CF₂CHF₂ | CHF₂ | NH₂ | trans-1,2-c-Pr- | 3-Cl | |
| 5-25 | CHF₂ | CHF₂ | 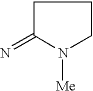 | trans-1,2-c-Pr- | — | |
| 5-26 | CFMe₂ | CHF₂ | 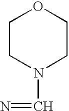 | trans-1,2-c-Pr- | — | |
| 5-27 | Me | 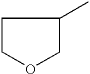 | NH₂ | trans-1,2-c-Pr- | — | |
| 5-28 | CHClMe | 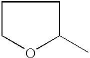 | NH₂ | trans-1,2-c-Pr- | — | |
| 5-29 | CCl₂Me | 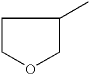 | NH₂ | trans-1,2-c-Pr- | — | |
| 5-30 | CF₂CF₃ | 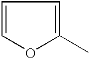 | NH₂ | trans-1,2-c-Pr- | 3,5-Me₂ | |
| 5-31 | CH₂CH₂—CF₃ | 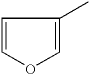 | NH₂ | trans-1,2-c-Pr- | 4-Cl | |
| 5-32 | CFMe₂ | Ph | NH₂ | trans-1,2-c-Pr- | 3-Cl | |
| 5-33 | CHFMe | Ph | NH₂ | trans-1,2-c-Pr- | 3-I | |
| 5-34 | CClMe₂ | 3,5-Cl₂— | NH₂ | trans-1,2-c-Pr- | 2,3-F₂ | |
| | | Ph | | | | |
| 5-35 | CFMe₂ | 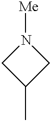 | NH₂ | trans-1,2-c-Pr- | — | |
| 5-36 | CFMeEt | 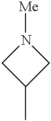 | NH₂ | trans-1,2-c-Pr- | — | |
| 5-37 | CClMe₂ | 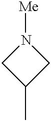 | NH₂ | trans-1,2-c-Pr- | — | |
| 5-38 | CHClMe | 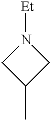 | NH₂ | trans-1,2-c-Pr- | — | |
| 5-39 | c-Pr | 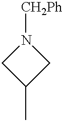 | NH₂ | trans-1,2-c-Pr- | — | |

TABLE 6
Compounds of the formula (1a)

| | | | | | | |
|---|---|---|---|---|---|---|
| | | | | | | Phys. |
| No. | R¹ | —A¹—R² | R³ | A²—Cy—A³ | (X)ₙ | Data |

| | | | | | |
|---|---|---|---|---|---|
| 6-1 | CH₂Cl | CHF₂ | NH₂ | trans- | — |
| | | | | 1,2-c-Pr-A1- | |
| 6-2 | CH₂OMe | CHF₂ | NHAc | trans- | — |
| | | | | 1,2-c-Pr-A1- | |
| 6-3 | CH₂OEt | CH₂Cl | NHCOEt | trans- | — |
| | | | | 1,2-c-Pr-A1- | |
| 6-4 | CFMe₂ | A1-c-Pr | NH₂ | trans- | 3,5-F₂ |
| | | | | 1,2-c-Pr-A1- | |
| 6-5 | CH₂Cl | 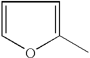 | NH₂ | cis- 1,2-c-Pr-A1- | 3-Cl |
| 6-6 | CF₂Me | c-Bu | 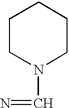 | cis- 1,2-c-Pr-A1- | — |
| 6-7 | CHFMe | c-Bu | 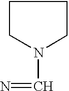 | cis- 1,2-c-Pr-A1- | — |
| 6-8 | CHClMe | c-Pr | 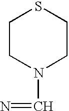 | cis- 1,2-c-Pr-A1- | 4-Cl |
| 6-9 | CHFMe | 2.2-Me₂— | NH₂ | cis- | 3,5-Me₂ |
| | | c-Pr | | 1,2-c-Pr-A1- | |
| 6-10 | 1-F-c-Pr | Et | NH₂ | cis- | — |
| | | | | 1,2-c-Pr-A1- | |
| 6-11 | CF₃ | Me | NH₂ | trans- | — |
| | | | | 1,2-c-Pr-A1- | |
| 6-12 | 3,5-F₂—Ph | c-Bu | NH₂ | trans- | — |
| | | | | 1,2-c-Pr-A1- | |
| 6-13 | CClMe₂ | 2,2-Me₂—c-Bu | 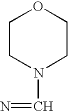 | trans- 1,2-c-Pr-A1- | — |
| 6-14 | CFMe₂ | c-Bu | NH₂ | trans- | — |
| | | | | 1,2-c-Pr-O- | |
| 6-15 | CFMe₂ | c-Pr | NH₂ | trans- | — |
| | | | | 1,2-c-Pr-O- | |
| 6-16 | CHFEt | Et | NH₂ | trans- | — |
| | | | | 1,2-c-Pr-O- | |
| 6-17 | CHFMe | Me | NH₂ | trans- | — |
| | | | | 1,2-c-Pr-O- | |
| 6-18 | CHFMe₂ | A1-c-Pr | NH₂ | trans- | — |
| | | | | 1,2-c-Pr-O- | |
| 6-19 | CFMe₂ | c-Pr | NH₂ | trans- | 3-Cl |
| | | | | 1,2-c-Pr-O- | |
| 6-20 | CF₃ | c-Bu | NH₂ | trans- | 3,5-Me₂ |
| | | | | 1,2-c-Pr-O- | |
| 6-21 | CFMe₂ | Me | NH₂ | cis- | 3-F |
| | | | | 1,2-c-Pr-O- | |
| 6-22 | CF₃ | Et | NH₂ | cis- | — |
| | | | | 1,2-c-Pr-O- | |
| 6-23 | CFMe₂ | Me | NH₂ | trans-1,2-Ox- | 3-Pr |
| 6-24 | 1-F-c-Pr | Me | NH₂ | trans-1,2-Ox- | 3-Cl |
| 6-25 | CHFEt | Me | NH₂ | cis-1,2-Ox- | — |
| 6-26 | CHFMe | Me | NH₂ | cis-1,2-Ox- | — |
| 6-27 | CClMe₂ | Et | NH₂ | -A1-cis-1,2-Ox- | — |
| 6-28 | i-Pr | Et | NH₂ | -A1-cis-1,2-Ox- | — |
| 6-29 | CH₂Cl | c-Pr | NH₂ | trans-1,2-Ox-A1 | — |
| 6-30 | CH₂F | c-Pr | NH₂ | trans-1,2-Ox-A1 | 3,5-Me₂ |
| 6-31 | CF₃ | c-Pr | NH₂ | cis-3,4-Az(Me)- | 4-Cl |
| 6-32 | CF₂Cl | i-Pr | NH₂ | cis-3,4-Az(Me)- | 3-Cl |
| 6-33 | CClF₂ | A1-c-Bu | NH₂ | cis-3,4-Az(Me)- | 3-I |
| 6-34 | CH₂Br | A1-c-Bu | NH₂ | trans-3,4- | 2,3-F₂ |
| | | | | Az(Me)- | |
| 6-35 | CFMe₂ | Me | N═C(Ph)— | trans-3,4- | — |
| | | | NMe₂ | Az(Me)- | |
| 6-36 | CFMe₂ | Me | NH₂ | cis-1,2-c-Bu- | — |
| 6-37 | CHFMe | c-Pr | NH₂ | cis-1,2-c-Bu- | — |
| 6-38 | CHFEt | c-Bu | NH₂ | cis-1,2-c-Bu- | — |
| 6-39 | CF₂CF₃ | A1-Ph | NH₂ | cis-1,2-c-Bu- | — |
| 6-40 | CHFMe | A1-3-Cl— | NH₂ | trans-1,2-c-Bu- | 3,5-Me₂ |
| | | Ph | | | |
| 6-41 | CFEt₂ | 2-Furyl | NH₂ | trans-1,2-c-Bu- | — |
| 6-42 | CFMeEt | 2-THF | NH₂ | trans-1,2-c-Bu- | — |
| 6-43 | CFMe₂ | 3-THF | NH₂ | trans-1,2-c-Bu- | — |
| 6-44 | Me | Et | NH₂ | trans-1,3-c-Bu- | 3-Me |
| 6-45 | Et | Pr | NH₂ | trans-1,3-c-Bu- | — |
| 6-46 | CFMe₂ | c-Pr | NH₂ | trans-1,3-c-Bu- | — |
| 6-47 | CFEt₂ | i-Pr | NHAc | trans-1,3-c-Bu- | — |
| 6-48 | CF₃ | c-Pr | NHCHO | trans-1,3-c-Bu- | — |
| 6-49 | CHF₂ | c-Bu | NHCHO | trans-1,3-c-Bu- | — |
| 6-50 | Et | Et | NHCOPr | trans-1,3-c-Bu- | — |
| 6-51 | CFMe₂ | Me | NH₂ | cis-1,2-c-Bu-O- | — |
| 6-52 | 1-F-c-Pr | Et | NH₂ | trans-1,2-c-Pen- | — |
| 6-53 | CFMe₂ | Me | NH₂ | trans-1,2-c-Pen- | — |
| 6-54 | CF₃ | i-Pr | NH₂ | trans-1,2-c-Pen- | — |
| 6-55 | CH₂Cl | c-Pr | NH₂ | cis-1,2-c-Pen- | — |
| 6-56 | CCl₃ | c-Pen | NH₂ | cis-1,2-c-Pen- | — |
| 6-57 | CBrCl₂ | c-Pen | NH₂ | cis-1,2-c-Pen- | — |
| 6-58 | CH₂F | CH₂F | NH₂ | trans-3,4-THF- | — |
| 6-59 | CCl₃ | CH₂Cl | NH₂ | trans-3,4-THF | — |
| 6-60 | 2-Furyl | CH₂Cl | NH₂ | trans-3,4-THF | — |
| 6-61 | 2-THF | CH₂Cl | NH₂ | trans-3,4-THF | — |
| 6-62 | 2-THF | Et | NH₂ | trans-3,4-THF | — |
| 6-63 | CFMe₂ | Et | NH₂ | trans-3,4-THF | — |
| 6-64 | CFMe₂ | A1-c-Pr | NH₂ | trans-3,4-THF | — |
| 6-65 | CFMe₂ | c-Bu | NH₂ | cis-2,3-THF- | — |
| 6-66 | CFMe₂ | Me | NH₂ | cis-2,3-THF- | — |
| 6-67 | CFMe₂ | Et | NH₂ | cis-2,3-THF- | — |
| 6-68 | CFEt₂ | Ph | NHCO-n- C₂₅H₁₀ | 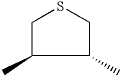 | — |
| 6-69 | CFEt₂ | c-Pr | NH₂ | 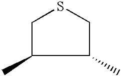 | — |
| 6-70 | CFEt₂ | c-Bu | NH₂ | 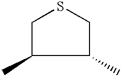 | — |
| 6-71 | CFEt₂ | 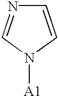 | NH₂ | 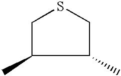 | — |
| 6-72 | CFEt₂ | 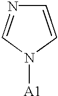 | NH₂ | 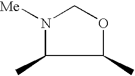 | — |
| 6-73 | CFMe₂ | 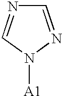 | NH₂ | 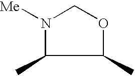 | 3-CN |
| 6-74 | CHF₂ | c-Pr | NH₂ | 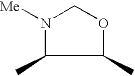 | 4-CN |
| 6-75 | CF₃ | c-Bu | NH₂ | 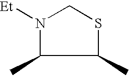 | 3-NO₂ |
| 6-76 | CF₂CF₃ | c-Bu | NH₂ | 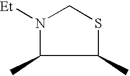 | 4-NO₂ |
| 6-77 | CF₂CHF₂ | c-Bu | NH₂ | 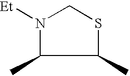 | 3-Cl |
| 6-78 | CF₃ | Me | NH₂ | 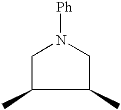 | — |
| 6-79 | CCl₃ | Me | NH₂ | 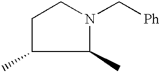 | — |
| 6-80 | CHF₂ | Pr | NHCHO | 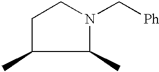 | — |
| 6-81 | Me | Me | NH₂ | trans-1,2-Hex- | — |
| 6-82 | CH₂F | Et | NH₂ | trans-1,2-Hex- | — |
| 6-83 | CH₂Cl | CH₂Cl | NH₂ | trans-1,2-Hex- | — |

TABLE 7
Compounds of the formula (1b)

| | | | | | | (1b) |
|---|---|---|---|---|---|---|
| 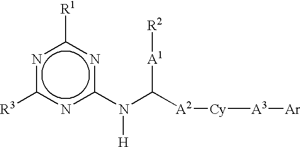 | | | | | | |
| | | | | | | Phys. |
| No. | R¹ | —A¹—R² | R³ | A²—Cy—A³ | Ar | Data |

| | | | | | |
|---|---|---|---|---|---|
| 7-1 | CFMe₂ | Et | NH₂ | trans-1,2-c- | Pyrid-2-yl |
| | | | | Pr- | |
| 7-2 | CHfMe | Et | NH₂ | trans-1,2-c- | Pyrid-3-yl |
| | | | | Pr- | |
| 7-3 | CHFEt | c-Bu | NH₂ | trans-1,2-c- | Pyrid-4-yl |
| | | | | Pr- | |
| 7-4 | CFMe₂ | c-Bu | NH₂ | trans-1,2-c- | Pyrimidin-2-yl |
| | | | | Pr- | |
| 7-5 | CF₃ | c-Pr | NH₂ | trans-1,2-c- | 4,6-Me₂- |
| | | | | Pr- | pyrimidin-2-yl |
| 7-6 | CHF₂ | c-Pen | NH₂ | trans-1,2-c- | Pyrimidin-4-yl |
| | | | | Pr- | |
| 7-7 | CF₃ | c-Pr | NH₂ | trans-1,2-c- | 2-Et-pyrimidin- |
| | | | | Pr- | 5-yl |
| 7-8 | CFMe₂ | Pr | NH₂ | trans-1,2-c- | Pyrimidin-2-yl |
| | | | | Pr- | |
| 7-9 | CHFEt | c-Bu | NH₂ | trans-1,2-c- | 4,6-Me₂-1,3,5- |
| | | | | Pr- | triazin-2-yl |
| 7-10 | CHFMe | c-Pr | NH₂ | trans-1,2-c- | 4,6-Me₂-1,3,5- |
| | | | | Pr- | triazin-2-yl |
| 7-11 | CHFMe | c-Bu | NH₂ | trans-1,2-c- | Thien-2-yl |
| | | | | Pr- | |
| 7-12 | CFMe₂ | Me | NH₂ | trans-1,2-c- | 5-Methyl-thien- |
| | | | | Pr- | 2-yl |
| 7-13 | CFMe₂ | Et | NH₂ | trans-1,2-c- | 5-Ethyl-thien- |
| | | | | Pr- | 2-yl |
| 7-14 | CFMe₂ | Et | NH₂ | cis-1,2-c- | Thien-3-yl |
| | | | | Pr- | |
| 7-15 | CFMe₂ | Me | NH₂ | cis-1,2-c- | Fur-2-yl |
| | | | | Pr- | |
| 7-16 | CFMe₂ | c-Pr | NH₂ | cis-1,2-c- | 5-Methyl-fur-2- |
| | | | | Pr- | yl |
| 7-17 | CHFEt | Et | NH₂ | cis-1,2-c- | 1,3-Isoxazol-2- |
| | | | | Pr- | yl |
| 7-18 | CHFEt | c-Bu | NH₂ | cis-1,2-c- | 1,3-Isoxazol-2- |
| | | | | Pr- | yl |
| 7-19 | CHFMe | c-Bu | NH₂ | cis-1,2-c- | 1,2-Thiazol-3- |
| | | | | Pr- | yl |
| 7-20 | CHFMe | c-Bu | NH₂ | cis-1,2-c- | 4-Methyl-1,2- |
| | | | | Pr- | thiazol-3-yl |

### B. FORMULATION EXAMPLES

a) A dust is obtained by mixing 10 parts by weight of a compound of the formula (I) and 90 parts by weight of talc as inert substance and comminuting the mixture in a hammer mill.

b) A wettable powder which is readily dispersible in water is obtained by mixing 25 parts by weight of a compound of the formula (I), 64 parts by weight of kaolin-containing quartz as inert material, 10 parts by weight of potassium lignosulfonate and 1 part by weight of sodium oleoylmethyltauride as wetter and dispersant and grinding the mixture in a pinned-disk mill.

c) A dispersion concentrate which is readily dispersible in water is obtained by mixing 20 parts by weight of a compound of the formula (I), 6 parts by weight of alkylphenol polyglycol ether ®Triton X 207), 3 parts by weight of isotridecanolpolyglycol ether (8 EO) and 71 parts by weight of paraffinic mineral oil (boiling range, for example, approx. 255 to above 277° C.) and grinding the mixture in a friction ball mill to a fineness of under 5 microns.

d) An emulsifiable concentrate is obtained from 15 parts by weight of a compound of the formula (I), 75 parts by weight of cyclohexanone as solvent and 10 parts by weight of ethoxylated nonylphenol as emulsifier.

e) Water-dispersible granules are obtained by mixing 75 parts by weight of a compound of the formula (I),

| | | |
|---|---|---|
| 75 | parts by weight | of a compound of the formula (I), |
| 10 | parts by weight | of calcium lignosulfonate, |
| 5 | parts by weight | of sodium lauryl sulfate, |
| 3 | parts by weight | of polyvinyl alcohol and |
| 7 | parts by weight | of kaolin, |

grinding the mixture in a pinned-disk mill and granulating the powder in a fluidized bed by spraying on water as granulating fluid.

f) Water-dispersible granules are also obtained by homogenizing and precomminuting

| | | |
|---|---|---|
| 25 | parts by weight | of a compound of the formula (I), |
| 5 | parts by weight | of sodium 2,2′-dinaphthylmethane-6,6′-disulfonate, |
| 2 | parts by weight | of sodium oleoylmethyltauride, |
| 1 | part by weight | of polyvinyl alcohol, |
| 17 | parts by weight | of calcium carbonate and |
| 50 | parts by weight | of water |

in a colloid mill, subsequently grinding the mixture in a bead mill and atomizing and drying the suspension thus obtained in a spray tower by is of a single-substance nozzle.

### C. BIOLOGICAL EXAMPLES

1. Pre-Emergence Herbicidal Action

Seeds and rhizome sections of monocotyledonous and dicotyledonous harmful plants are placed in sandy loam in plastic pots and covered with soil. The compounds according to the invention which are formulated in the form of wettable powders or emulsion concentrates at various rates, are then applied to the surface of the covering soil as aqueous suspension or emulsion at an application rate of 600 to 800 l of water per ha (converted).

After the treatment, the pots are placed in the greenhouse and kept under good growth conditions for the weeds. Visual scoring of the plant damage or the adverse effect on emergence is done after the test plants have emerged after an experimental period of 3 to 4 weeks in comparison with untreated controls. As demonstrated by the test results, the compounds according to the invention have a good herbicidal pre-emergence activity against a broad spectrum of grass weeds and broad-leafed weeds. For example, the Examples No. 1-1, 1-2, 1-3, 1-4, 1-6, 2-1, 2-2, 2-3, 2-8, 3-1, 3-2, 3-3, 3-4, 3-26, 3-27, 3-28, 4-1, 4-2, 4-3, 4-4, 4-27, 4-28, 5-1, 5-2, 5-3 and 5-4 (see Tables 1 to 7) exhibit a very good herbicidal activity in the test against harmful plants such as Stellaria media, Lolium multiflorum, Amaranthus retroflexus, Sinapis alba, Avena sativa and Setaria viridis when applied during pre-emergence at an application rate of 1 kg or less active substance per hectare.

2. Post-Emergence Herbicidal Action

Seeds or rhizome sections of monocotyledonous and dicotyledonous weeds are placed in sandy loam soil in plastic pots, covered with soil and grown in the greenhouse under good growth conditions. Three weeks after sowing, the experimental plants are treated at the three-leaf stage. The compounds according to the invention, which are formulated as wettable powders or as emulsion concentrates, are sprayed at various rates onto the green plant organs at an application rate of 600 to 800 l of water per ha (converted). After the experimental plants have remained in the greenhouse for approx. 3 to 4 weeks under optimal growth conditions, the effect of the compositions is scored visually in comparison with untreated controls. The substances according to the invention also have a good herbicidal action against a broad spectrum of economically important grass weeds and broad-leaved weeds when applied post-emergence. For example, the Examples No. 1-1, 1-2, 1-3, 1-4, 1-6, 2-1, 2-2, 2-3, 2-8, 3-1, 3-2, 3-3, 3-4, 3-26, 3-27, 3-28, 4-1, 4-2, 4-3, 4-4, 4-27, 4-28, 5-1, 5-2, 5-3 and 54 (see Tables 1 to 7) exhibit a very good herbicidal activity in the test against harmful plants such as Sinapis alba, Echinochloa crus-galli, Lolium multiflorum, Stellaria media, Cyperus iria, Amaranthus retroflexus, Setaria viridus and Avena sativa when applied post-emergence at an application rate of 1 kg and less of active substance per hectare.

3. Activity Against Harmful Plants in Rice

Transplanted and seeded rice and typical grass weeds and broad-leaved weeds in rice crops are grown in the greenhouse under paddy rice conditions (depth of the paddy water: 2-3 cm) in sealed plastic pots until they have reached the three-leaf stage (Echinochloa crus-galli 1.5-leaf). They are then treated with the compounds according to the invention. To this end, the formulated active substances are suspended, dissolved or emulsified in water and the product is poured into the paddy water of the test plants at various dosages. After the treatment has been carried out thus, the test plants are placed in the greenhouse under optimal growth conditions and kept like this over the entire experimental period.

About three weeks after application, the evaluation is carried out by is of visually scoring the plant damage in comparison with untreated controls. The compounds according to the invention exhibit a very good herbicidal activity against harmful plants. For example, the compounds of Examples No. 1-1, 1-2, 1-3, 14, 1-6, 2-1, 2-2, 2-3, 2-8, 3-1, 3-2, 3-3, 3-4, 3-26, 3-27, 3-28, 4-1, 4-2, 4-3, 4-4, 4-27, 4-28, 5-1, 5-2, 5-3 and 5-4 (see Tables 1 to 7) exhibit, in the test, a very good herbicidal activity against harmful plants which are typical of rice crops such as, for example, Cyperus monti, Echinochloa crus-galli and Sagittaria pygmaea.

4. Crop Plant Tolerance

In further greenhouse tests, seeds of a substantial number of crop plants and harmful plants are placed in sandy loam soil and covered with soil. Some of the pots are immediately treated as described under item 1, while the remaining plants are placed in the greenhouse until the plants have developed two to three true leaves and then sprayed as described under item 2 with the substances of the formula (I) according to the invention at various rates. Four to five weeks after the application and standing time in the greenhouse, it is found by is of visually scoring that the compounds according to the invention leave dicotyledonous crops such as, for example, soybean, cotton, oilseed rape, sugarbeet and potatoes undamaged pre and post-emergence, even at high doses of active substance. In addition, some substances also leave graminaceous crops, such as, for example, barley, wheat, rye, sorghum, maize or rice, unharmed. The compounds of the formula (I) show a high selectivity in some cases and are therefore suitable for controlling undesired vegetation in agricultural crops.

We claim:

1. A compound of the formula (I) or a salt thereof, 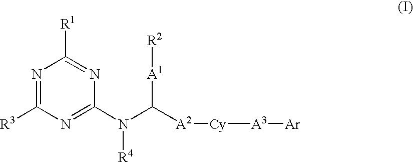
where
R¹ is aryl, aryloxy, arylthio, arylamino, N-aryl-N—(C₁-C₄)alkylamino, (C₃-C₉)cycloalkyl, (C₃-C₉)cycloalkyloxy, (C₃-C₉)cycloalkylthio, (C₃-C⁹)cycloalkylamino, N—(C₃-C₉)cycloalkyl-N—(C₁-C₄)alkylamino, di-[(C₃-C₉)cycloalkyl]amino, heterocyclyl, heterocyclyloxy, heterocyclylthio, heterocyclylamino or N-heterocyclyl-N—(C₁-C₄)alkyl-amino, it being possible for each of the last-mentioned 16 to be unsubstituted or substituted, or
hydrogen, (C₁-C₆)alkyl, (C₂-C₆)alkenyl, (C₂-C₆)alkynyl, (C₁-C₆)alkoxy, (C₂-C₆)alkenyloxy, (C₂-C₆)alkynyloxy, (C₁-C₆)alkylthio, (C₂-C₆)alkenylthio, (C₂-C₆)alkynylthio, (C₁-C₆)alkylamino or di[(C₁-C₆)alkyl]amino,
where each of the last-mentioned 11 radicals is unsubstituted or substituted by one or more radicals selected from the group consisting of halogen, hydroxyl, cyano, nitro, thiocyanato, (C₁-C₄)alkoxy, (C₁-C₄)haloalkoxy, (C₂-C₄)alkenyloxy, (C₂-C₄)haloalkenyloxy, (C₁-C₄)alkylthio, (C₁-C₄)alkylsulfinyl, (C₁-C₄)alkylsulfonyl, (C₁-C₄)haloalkylthio, (C₁-C₄)haloalkylsulfinyl, (C₁-C₄)haloalkylsulfonyl, (C₃-C₉)cycloalkyl which is unsubstituted or substituted, phenyl which is unsubstituted or substituted, heterocyclyl which is unsubstituted or substituted, and radicals of the formulae R′—C(═Z′)—, R′—C(═Z′)—Z—, R′—Z—C(═Z′)—, R′R″N—C(═Z′)—, R′—Z—C(═Z′)—O—, R′R″N—C(═Z′)—Z—, R′—Z—C(═Z′)—NR″— and R′R″N—C(═Z′)—NR′″—,
where R′, R″ and R′″ in each case independently of one another are (C₁-C₆)alkyl, aryl, aryl(C₁-C₆)alkyl, (C₃-C₉)cycloalkyl or (C₃-C₉)cycloalkyl(C₁-C₆)alkyl, where each of the last-mentioned 5 radicals is unsubstituted or substituted and in which Z and Z′ independently of one another are in each case an oxygen or sulfur atom,
and, including substituents, preferably has 1 to 30 carbon atoms,
R² is (C₁-C₆)alkyl, (C₂-C₆)alkenyl, (C₂-C₆)alkynyl, where each of the last-mentioned three radicals is unsubstituted or substituted, (C₃-C₉)cycloalkyl which is unsubstituted or substituted, (C₄-C₉)cycloalkenyl which is unsubstituted or substituted, heterocyclyl which is unsubstituted or substituted, or phenyl which is unsubstituted or substituted, where R² including substituents preferably has up to 30 carbon atoms,
R³ is hydrogen, (C₁-C₆)alkyl, aryl or (C₃-C₉)cycloalkyl, where each of the last-mentioned 3 radicals is unsubstituted or substituted, or is a radical of the formula —N(B¹—D¹)(B²—D²) or —NR′—N(B¹—D¹)(B²—D²), where B¹, B², D¹ and D² are in each case as defined hereinbelow and R′ is hydrogen, (C₁-C₆)alkyl or [(C₁-C₄)alkyl]carbonyl,
or is a group of the formula 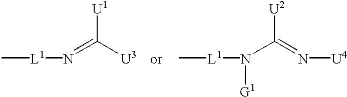
where
L¹ is a direct bond, —O—, —S— or a group of the formula —NG²—,
U¹, U² independently of one another are a group of the formula G³, OG⁴, SG⁵, NG⁶G⁷, NG⁸NG⁹G¹⁰, NG¹¹OG ¹²or NG¹¹SG¹²,
U³ is a group of the formula G¹³, OG¹⁴, SG¹⁵, NG¹⁶G¹⁷, NG¹⁸NG¹⁹G²⁰, NG²¹OG²² or NG²³SG²⁴,
U⁴ is a group of the formula G²⁵, OG²⁶, SG²⁷ or NG²⁸G²⁹,
where the radicals G¹ to G²⁹ independently of one another are hydrogen, aryl which is unsubstituted or substituted or (C₃-C₉)cycloalkyl which is unsubstituted or substituted or heterocyclyl which is substituted or unsubstituted or
(C₁-C₆)alkyl, (C₂-C₆)alkenyl or (C₂-C₆)alkynyl,
where each of the last-mentioned 3 radicals is unsubstituted or substituted by one or more radicals selected from the group consisting of halogen, hydroxyl, cyano, nitro, thiocyanato, (C₁-C₄)alkoxy, (C₁-C₄)haloalkoxy, (C₂-C₄)alkenyloxy, (C₂-C₄)haloalkenyloxy, (C₁-C₄)alkylthio, (C₁-C₄)alkylsulfinyl, (C—C₄)alkylsulfonyl, (C₁-C₄)haloalkylthio, (C₁-C₄)haloalkylsulfinyl, (C₁-C₄)haloalkylsulfony l, (C₃-C₉)cycloalkyl which is unsubstituted or substituted, phenyl which is unsubstituted or substituted and heterocyclyl which is unsubstituted or substituted,
or the radicals U¹ and U³ or U² and U⁴ or U² and G¹ or U⁴ and G¹ pairwise with the atoms linking them are in each case a carbocyclic or heterocyclic ring with 4 to 7 ring atoms, the ring being unsubstituted or substituted,
R⁴ is a radical of the formula —B³—D³, where B³ and D³ are as defined hereinbelow,
Ar is aryl, (C₃-C₉)cycloalkyl, (C₄-C₉)cycloalkenyl or heterocyclyl, where each of the last-mentioned 4 radicals is unsubstituted or substituted by (X)ₙ where (X)ₙ is in each case
n substituents X and where X in each case independently of one another are halogen, hydroxyl, amino, nitro, formyl, carboxyl, cyano, thiocyanato, aminocarbonyl or (C₁-C₆)alkyl, (C₁-C₆)alkoxy, (C₁-C₆)alkylthio, mono(C₁-C₆)alkylamino, di(C₁-C₄)alkylamino, (C₂-C₆)alkenyl, (C₂-C₆)alkynyl, [(C₁-C₆)alkyl]carbonyl, [(C₁-C₆)alkoxy]carbonyl, mono(C₁-C₆)alkylaminocarbonyl, di(C₁-C₄)alkylaminocarbonyl, N-(C₁-C₆)alkanoylamino, N—(C₁-C₄)alkanoyi-N—(C₁-C₄)alkylamino or N,N-di(C₁-C₄)alkanoylamino,
where each of the last-mentioned 14 radicals is unsubstituted or substituted,
or (C₃-C₉)cycloalkyl, (C₃-C₉)cycloalkoxy, (C₃-C₉)cycloalkylamino, phenyl, phenoxy, phenylthio, phenylcarbonyl, phenylamino, heterocyclyl, heterocyclyloxy, heterocyclylthio or heterocyclylamino,
where each of the last-mentioned 12 radicals is unsubstituted or substituted,
or two adjacent radicals X together form a fused cycle with 4 to 6 ring atoms which is carbocyclic or contains hetero ring atoms selected from the group consisting of O, S and N and which is unsubstituted or substituted by one or more radicals selected from the group consisting of halogen, (C₁-C₄)alkyl and oxo, and
n is 0, 1, 2, 3, 4 or 5,
A¹ and A² in each case independently of one another are a direct bond or straight-chain alkylene having 1 to 4 carbon atoms or straight-chain alkenylene or alkynylene having in each case 2 to 5 carbon atoms, where each of the last-mentioned three diradicals is unsubstituted or substituted by one or more radicals selected from the group consisting of halogen, nitro, cyano, thiocyanato and radicals of the formula —B⁴—D⁴, or are a divalent radical of the formula V¹, V², V³, V⁴ or V⁵,
—CR^{a}R^{b}—W*—CR^{c}R^{d}— (V¹)
—CR^{a}R^{b}—W*—CR^{c}R^{d}—CR^{e}R^{f}— (V²)
—CR^{a}R^{b}—CR^{c}R^{d}—W*—CR^{e}R^{f}— (V³)
—CR^{a}R^{b}—CR^{c}R^{d}—W*— (V⁴)
—CR^{a}R^{b}—W*— (V⁵)
where each of the radicals R^{a}, R^{b}, R^{c}, R^{d}, R^{e} and R^{f} in each case independently of one another is hydrogen, halogen, nitro, cyano, thiocyanato or a radical of the formula —B⁵—D⁵,
W* is in each case an oxygen atom, a sulfur atom or a group of the formula N(B⁶—D⁶) and
B⁴, B⁵, B⁶, D⁴, D⁵ and D⁶ are as defined hereinbelow,
A³ is a direct bond or straight-chain alkylene having 1 to 4 carbon atoms, or straight-chain alkenylene or alkynylene having in each case 2 to 5 carbon atoms, each of the three last-mentioned diradicals being unsubstituted or substituted by one or more radicals selected from the group consisting of halogen, nitro, cyano, thiocyanato and radicals of the formula —B⁷—D⁷, or is a divalent radical of the formula W¹, W², W³, W⁴, W⁵, W⁶, W⁷ or W⁸,
—CR^{aa}R^{bb}—W**—CR^{cc}R^{dd}—(W¹)
—CR^{aa}R^{bb}—W**—CR^{cc}R^{dd}—CR^{ee}R^{ff}— (W²)
—CR^{aa}R^{bb}—CR^{cc}R^{dd}—W**—CR^{ee}R^{ff}— (W³)
—CR^{aa}R^{bb}—CR^{cc}R^{dd}—W**— (W⁴)
—CR^{aa}R^{bb}—W**— (W⁵)
—W **—CR^{aa}R^{bb}—CR^{cc}R^{dd}— (W⁶)
—W**—CR^{aa}R^{bb}— (W⁷)
—W**— (W⁸)
where each of the radicals R^{aa}, R^{bb}, R^{cc}, R_{dd}, R^{ee} and R^{ff} in each case independently of one another is hydrogen, halogen, nitro, cyano, thiocyanato or a radical of the formula —B⁸—D⁸,
W** is in each case an oxygen atom, a sulfur atom or a group of the formula N(B⁹—D⁹) and
B⁷, B⁸, B⁹, D⁷, D⁸ and D⁹ are as defined hereinbelow,
B¹, B², B³, B⁶ and B⁹ in each case independently of one another are a direct bond or are a divalent group of the formulae —C(═Z*)—, —C(═Z*)—Z**—, —C(═Z*)—NH— or —C(═Z*)—NR*—, where Z* is an oxygen or sulfur atom, Z** an oxygen or sulfur atom and R* (C₁-C₆)alkyl, aryl, aryl(C₁-C₆)alkyl, (C₃-C₉)cycloalkyl or (C₃-C₉)cycloalkyl(C₁-C₆)alkyl, where of the 5 last-mentioned radicals is unsubstituted or substituted,
B⁴, B⁵, B⁷ and B⁸ in each case independently of one another are a direct bond or a divalent group of the formulae —O—, —S(O)ₚ—, —S(O)ₚ—O—, —O—S(O)ₚ—, —CO—, —O—CO—, —CO—O—, —S—CO—, —CO—S—, —S—CS—, —CS—S—, —O—CO—O—, —NR^{o}—, —O—NR^{o}—, —NR^{o}—O—, —NR^{o}—CO—, —CO—NR^{o}—, —O—CO—NR^{o}— or —NR^{o}—CO—O—, where p is in each case the integer 0, 1 or 2 and R^{o} is in each case hydrogen, (C₁-C₆)alkyl, aryl, aryl(C₁-C₆)alkyl, (C₃-C₉)cycloalkyl or (C₃-C₉)cycloalkyl(C₁-C₆)alkyl, where each of the 5 last-mentioned radicals is unsubstituted or substituted,
D¹, D², D³, D⁴, D⁵, D⁶, D⁷, D⁸ and D⁹ in each case independently of one another are hydrogen, (C₁-C₆)alkyl, aryl, aryl(C₁-C₆)alkyl, (C₃-C₉)cycloalkyl or (C₃-C₉)cycloalkyl(C₁-C₆)alkyl, where each of the 5 last-mentioned radicals is unsubstituted or substituted,
Cy is a saturated carbocyclic ring with 3 to 8 ring atoms which is unsubstituted or substituted, or a saturated heterocyclic ring with 3 to 8 ring atoms and one to three hetero ring atoms selected from the group consisting of N, O, S and P, the ring being unsubstituted or substituted and, in the event of the substitution on the nitrogen atom as hetero ring atom, the substituent being oxygen or a group of the formula —B′—D′, and in the case of substitution on the hetero ring atom phosphorus, the phosphorus atom together with the substituents forming a divalent phosphorus group of the formula —P(═O)(B″—D″)— or —P(═S)(B″—D″)—, where B′ and B″ independently of one another are as defined under B¹ and where D′ and D″ independently of one another are as defined under D¹.

2. A compound of the formula (I) or a salt thereof as claimed in claim 1, wherein
R¹ is phenyl, phenoxy, phenylthio, phenylamino, N-phenyl-N—(C₁-C₄)alkylamino, where each of the last-mentioned 5 radicals unsubstituted or substituted by one or more radicals selected from the group consisting of halogen, hydroxyl, amino, nitro, formyl, carboxyl, sulfo, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl and (C₁-C₄)haloalkylsulfonyl and, including substituents, has 6 to 30 carbon atoms, or
(C₃-C₉)cycloalkyl which is unsubstituted or substituted by one or more radicals selected from the group consisting of halogen, hydroxyl, amino, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino and di(C₁-C₄)alkylamino and, including substituents, has 3 to 30 carbon atoms or
heterocyclyl which is unsubstituted or substituted by one or more radicals selected from the group consisting of halogen, hydroxyl, amino, nitro, formyl, carboxyl, sulfonyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl and (C₁-C₄)haloalkylsulfonyl and, including substituents, has 2 to 30 carbon atoms, or
hydrogen, (C₁-C₆)alkyl, (C₂-C₆)alkenyl, (C₂-C₆)alkynyl, (C₁-C₆)alkoxy or (C₁-C₆)alkylthio,
where each of the last-mentioned 5 radicals is unsubstituted or substituted by one or more radicals selected from the group consisting of halogen, hydroxyl, cyano, nitro, thiocyanato, (C₁-C₄)alkoxy, (C₁-C₄)haloalkoxy, (C₂-C₄)alkenyloxy, (C₂-C₄)haloalkenyloxy, (C₁-C₄)alkylthio, (C₁-C₄)alkylsulfinyl, (C₁-C₄)alkylsulfonyl, (C₁-C₄)haloalkylsulfinyl, (C₁-C₄)haloalkylsulfonyl and
(C₃-C₆)cycloalkyl, which is unsubstituted or substituted by one or more radicals selected from the group consisting of halogen, hydroxyl, amino, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino and di(C₁-C₄)alkylamino, and phenyl and heterocyclyl, where each of the two last-mentioned radicals is unsubstituted or substituted by one or more radicals selected from the group consisting of halogen, hydroxyl, amino, nitro, formyl, carboxyl, sulfonyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl and (C₁-C₄)haloalkylsulfonyl, and radicals of the formula R′—C(═Z′)—, R′—C(═Z′)—Z—, R′—Z—C(═Z′)—, R′R″N—C(═Z′)—, R′—Z—C(═Z′)—O—, R′R″N—C(═Z′)—Z—, R′—Z—C(═Z′)—NR″— and R′R″N—C(═Z′)—NR′″—,
where R′, R″ and R′″ in each case independently of one another are (C₁-C₄)alkyl, phenyl, phenyl(C₁-C₄)alkyl, (C₃-C₆)cycloalkyl or (C₃-C₆)cycloalkyl(C₁-C₄)alkyl, where each of the 5 last-mentioned radicals is unsubstituted or substituted by one or more radicals selected from the group consisting of halogen, hydroxyl, amino, nitro, formyl, cyano, thiocyanato, (C₁-C₄)alkoxy, (C₁-C₄)alkylthio, mono(C₁-C₄)alkylamino, di(C₁-C₄)alkylamino, (C₂-C₄)alkenyl, (C₂-C₄)alkynyl, (C₃-C₆)cycloalkyl and, in the case of cyclic radicals, also (C₁-C₄)alkyl and (C₁-C₄)haloalkyl, and where Z and Z′ independently of one another are in each case an oxygen or sulfur atom,
and, including substituents, preferably has 1 to 20 carbon atoms.

3. A compound of the formula (I) or a salt thereof as claimed in claim 1, wherein
R² is (C₁-C₆)alkyl, (C₂-C₆)alkenyl, (C₂-C₆)alkynyl, (C₃-C₉)cycloalkyl, (C₄-C₉)cycloalkenyl, phenyl or heterocyclyl (“Basic radicals” for R²), where each of the last-mentioned 7 radicals is unsubstituted or substituted by one or more radicals selected from the group consisting of the radicals A), B) and C), where
Group A) consists of the radicals halogen, hydroxyl, amino, nitro, formyl, carboxyl, aminocarbonyl, aminosulfonyl, sulfo, cyano, thiocyanato and oxo,
Group B) consists of the radicals (C₁-C₆)alkoxy, (C₁-C₆)alkylthio, mono(C₁-C₆)alkylamino, di(C₁-C₄)alkylamino, (C₃-C₉)cycloalkyl, (C₄-C₉)cycloalkenyl, radicals of the formulae R′—C(═Z′)—, R′—C(═Z′)—Z—, R′—Z—C(═Z′)—, R′R″N—C(═Z′)—, R′—Z—C(═Z′)—O—, R′R″N—C(═Z′)—Z—, R′—Z—C(═Z′)—NR″— and R′R″N—C(═Z′)—NR′″—, where R′, R″ and R′″ in each case independently of one another are (C₁-C₆)alkyl, phenyl, phenyl(C₁-C₆)alkyl, (C₃-C₉)cycloalkyl or (C₃-C₉)cycloalkyl(C₁-C₆)alkyl and where Z and Z′ independently of one another are in each case an oxygen or sulfur atom, and, in the case of cyclic basic radicals for R² also (C₁-C₆)alkyl, (C₂-C₆)alkenyl, (C₂-C₆)alkynyl, and, in the case of nonaromatic cyclic basic radicals for R², also (C₁-C₆)alkylidene and (C₄-C₉)cycloalkylidene,
Group C) consists of radicals as defined for group B), but where each radical is substituted by one or more radicals selected from the group consisting of halogen, hydroxyl, amino, nitro, formyl, carboxyl, sulfo, cyano, thiocyanato, aminocarbonyl, (C₁-C₄)alkoxy, (C₁-C₄)alkylthio, mono(C₁-C₄)alkylamino, d i(C₁-C₄)alkylamino, (C₃-C₉)cycloalkyl, (C₄-C₉)cycloalkenyl, [(C₁-C₄)alkyl]carbonyl, [(C₁-C₄)alkoxy]carbonyl, mono(C₁-C₄)alkylaminocarbonyl, di(C₁-C₄)alkylaminocarbonyl, phenyl, phenoxy, phenylthio, phenylamino, phenylcarbonyl, heterocyclyl, heterocyclyloxy, heterocyclylthio and heterocyclylamino and, in the case of cyclic radicals B), also (C₁-C₄)alkyl, and, in the case of nonaromatic cyclic radicals B), also (C₁-C₆)alkylidene and (C₄-C₉)cycloalkylidene,
where each of the last-mentioned 22 radicals is unsubstituted or substituted by one or more radicals selected from the group consisting of halogen, nitro, cyano, (C₁-C₄)alkoxy, (C₁-C₄)alkylthio, (C₁-C₄)haloalkoxy, formyl, (C₁-C₄)alkylcarbonyl and (C₁-C₄)alkoxycarbonyl and, in the case of cyclic radicals, also (C₁-C₄)alkyl, (C₁-C₄)alkoxy(C₁-C₄)alkyl and (C₁-C₄)haloalkyl and, in the case of nonaromatic cyclic radicals, also (C₁-C₆)alkylidene and (C₄-C₉)cycloalkylidene,
and including substituents has preferably 3 to 20 carbon atoms, and
A¹ is a direct bond or a group of the formula —CH₂—, —CH₂CH₂—, —CH₂CH₂CH₂— or —CH₂CH₂CH₂CH₂, where each of the 4 last-mentioned diradicals is unsubstituted or substituted by one or more radicals selected from the group consisting of halogen, nitro, cyano, thiocyanato and radicals of the formula —B⁴—D⁴, or is a divalent radical of the formula V¹, V², V³, V⁴ or V⁵,
—CR^{a}R^{b}—W*—CR^{c}R^{d}— (V¹)
—CR^{a}R^{b}—W*—CR^{c}R^{d}—CR^{e}R^{f}— (V²)
—CR^{a}R^{b}—CR^{c}R^{d}—W*—CR^{e}R^{f}— (V³)
—CR^{a}R^{b}—CR^{c}R^{d}—W*— (V⁴)
—CR^{a}R^{b}—W*— (V⁵)
where each of the radicals R^{a}, R^{b}, R^{c}, R^{d}, R^{e} and R^{f} in each case independently of one another is hydrogen, halogen, nitro, cyano, thiocyanato or a radical of the formula —B⁵—D⁵,
W* is in each case an oxygen atom, a sulfur atom or a group of the formula N(B⁶—D⁶) and
B⁴, B⁵ independently of one another are in each case a direct bond or a divalent group of the formulae —O—, —S(O)ₚ—, —S(O)ₚ—O—, —O—S(O)ₚ—, —CO—, —O—CO—, —CO—O—, —S—CO—, —CO—S—, —S—CS—, —CS—S—, —O—CO—O—, —NR^{o}—, —O—NR^{o}—, —NR^{o}—O—, —NR^{o}—CO—, —CO—NR^{o}—, —O—CO—NR^{o}— or —NR^{o}—CO—O—, where p is the integer 0, 1 or 2 and R° is hydrogen, (C₁-C₆)alkyl, aryl, aryl(C₁-C₆)alkyl, (C₃-C₉)cycloalkyl or (C₃-C₉)cycloalkyl(C₁-C₆)alkyl, where each of the 5 last-mentioned radicals is unsubstituted or substituted and, including substituents, preferably has up to 20 carbon atoms,
B⁶ is a direct bond or a divalent group of the formulae —C(═Z*)—, —C(═Z*)—Z**—, —C(═Z*)—NH— or —C(═Z*)—NR*—,
where Z*=an oxygen or sulfur atom, Z**=an oxygen or sulfur atom and R*═(C₁-C₆)alkyl, aryl, aryl(C₁-C₆)alkyl, (C₃-C₉)cycloalkyl or (C₃-C₉)cycloalkyl(C₁-C₆)alkyl,
where each of the 5 last-mentioned radicals is unsubstituted or substituted by one or more radicals selected from the group consisting of halogen, hydroxyl, amino, nitro, formyl, carboxyl, sulfo, cyano, thiocyanato, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl, (C₁-C₄)haloalkylsulfonyl and, in the case of cyclic radicals, also (C₁-C₄)alkyl and (C₁-C₄)haloalkyl and, including substituents, preferably have up to 20 carbon atoms,
D⁴, D⁵, D⁶ independently of one another are in each case hydrogen, (C₁-C₆)alkyl, aryl, aryl(C₁-C₆)alkyl, (C₃-C₉)cycloalkyl or (C₃-C₉)cycloalkyl(C₁-C₆)alkyl, where each of the 5 last-mentioned radicals is unsubstituted or substituted by one or more radicals selected from the group consisting of halogen, hydroxyl, amino, nitro, formyl, carboxyl, sulfo, cyano, thiocyanato, (C₁-C₄)Alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl, (C₁-C₄)haloalkylsulfonyl and, in the case of cyclic radicals, also (C₁-C₄)alkyl and (C₁-C₄)haloalkyl and, including substituents, preferably have up to 20 carbon atoms.

4. A compound of the formula (I) or a salt thereof as claimed in claim 1, wherein
R⁴ is hydrogen, (C₁-C₄)alkyl, phenyl or (C₃-C₆)cycloalkyl, where each of the 3 last-mentioned radicals is unsubstituted or substituted by one or more radicals selected from the group consisting of halogen, hydroxyl, amino, nitro, formyl, carboxyl, sulfonyl, cyano, thiocyanato, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₉)cycloalkyl, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, (C₁-C₄)alkylsulfonyl, (C₁-C₄)haloalkylsulfonyl and, in the case of cyclic radicals, also (C₁-C₄)alkyl and (C₁-C₄)haloalkyl, or formyl, [(C₁-C₄)alkyl]carbonyl, [(C₁-C₄)alkoxy]carbonyl, aminocarbonyl, mono(C₁-C₄)alkylaminocarbonyl or di(C₁-C₄)alkylaminocarbonyl.

5. A compound of the formula (I) or a salt thereof as claimed in claim 1, wherein
A² is a direct bond or a group of the formula
—CH₂—, —CH₂CH₂—, —CH₂CH₂CH₂—, —CH₂CH₂CH₂CH₂,
—CH₂—O—CH₂—, —CH₂—O—CH₂—CH₂—, —CH₂—CH₂—O—CH₂—,
—CH₂—S—CH₂—, —CH₂—S—CH₂—CH₂—, —CH₂—CH₂—S—CH₂—,
—CH₂—N H—CH₂—, —CH₂—NH—CH₂—CH₂—, —CH₂—CH₂—NH—CH₂—,
—CH₂—N(CH₃)—CH₂—, —CH₂—N(CH₃)—CH₂—CH₂— or —CH₂—CH₂—N(CH₃)—CH₂—.
A³ is a direct bond or a group of the formula
—O—, —S—, —CH₂—, —CH₂CH₂—, —CH₂CH₂CH₂—, —CH₂CH₂CH₂CH₂—,
—O—CH₂—, —CH₂—O—, —SCH₂— or —CH₂—S—,
Cy is a divalent (C₃-C₉)cycloalkylene group which is unsubstituted or substituted by one or more radicals selected from the group consisting of the radicals A^{c}), B^{c}) and C^{c}), where
Group A^{c}) consists of the radicals halogen, hydroxyl, amino, nitro, formyl, carboxyl, aminocarbonyl, aminosulfonyl, sulfo, cyano, thiocyanato and oxo,
Group B^{c}) consists of the radicals (C₁-C₆)alkyl, (C₂-C₆)alkenyl, (C₂-C₆)alkynyl, (C₁-C₆)alkylidene, (C₄-C₉)cycloalkylidene(C₁-C₆)alkoxy, (C₁-C₆)alkylthio, mono(C₁-C₆)alkylamino, di(C₁-C₄)alkylamino, (C₃-C₉)cycloalkyl, (C₄-C₉)cycloalkenyl, radicals of the formulae R′—C(═Z′)—, R′—C(═Z′)—Z—, R′—Z—C(═Z′)—, R′R″N—C(═Z′)—, R′—Z—C(═Z′)—O—, R′R″N—C(═Z′)—Z—, R′—Z—C(═Z′)—NR″— and R′R″N—C(═Z′)—NR′″—,
where R′, R″ and R′″ in each case independently of one another are (C₁-C₆)alkyl, phenyl, phenyl(C₁-C₆)alkyl, (C₃-C₉)cycloalkyl or (C₃-C₉)cycloalkyl(C₁-C₆)alkyl and where Z and Z′ independently of one another are in each case an oxygen or sulfur atom,
Group C^{c}) consists of radicals as defined for group B^{c}), but where each radical is substituted by one or more radicals selected from the group consisting of halogen, hydroxyl, amino, nitro, formyl, carboxyl, sulfo, cyano, thiocyanato, aminocarbonyl, (C₁-C₄)alkoxy, (C₁-C₄)alkylthio, mono(C₁-C₄)alkylamino, di(C₁-C₄)alkylamino, (C₃-C₉)cycloalkyl, (C₄-C₉)cycloalkenyl, [(C₁-C₄)alkyl]carbonyl, [(C₁-C₄)alkoxy]carbonyl, mono(C₁-C₄)alkylaminocarbonyl, di(C₁-C₄)alkylaminocarbonyl, phenyl, phenoxy, phenylthio, phenylamino, phenylcarbonyl, heterocyclyl, heterocyclyloxy, heterocyclylthio and heterocyclylamino and, in the case of cyclic radicals BC), also (C₁-C₄)alkyl, and, in the case of nonaromatic cyclic radicals BC), also (C₁-C₆)alkylidene and (C₄-C₉)cycloalkylidene,
where each of the last-mentioned 22 radicals is unsubstituted or substituted by one or more radicals selected from the group consisting of halogen, nitro, cyano, (C₁-C₄)alkoxy, (C₁-C₄)alkylthio, (C₁-C₄)haloalkoxy, formyl, (C₁-C₄)alkylcarbonyl and (C₁-C₄)alkoxycarbonyl and, in the case of cyclic radicals, also (C₁-C₄)alkyl, (C₁-C₄)alkoxy(C₁-C₄)alkyl and (C₁-C₄)haloalkyl and, in the case of nonaromatic cyclic radicals, also (C₁-C₆)alkylidene and (C₄-C₉)cycloalkylidene,
and where Cy including substituents has 3 to 20 carbon atoms.

6. A compound of the formula (I) or a salt thereof as claimed in claim 1, wherein
Ar is phenyl, (C₃-C₉)cycloalkyl, (C₄-C₉)cycloalkenyl or heterocyclyl, where each of the last-mentioned 4 radicals is in each case unsubstituted or substituted by (X)ₙ; and
(X)ₙ is n substituents X, where the substituents X independently of one another are halogen, hydroxyl, amino, nitro, formyl, carboxyl, cyano, thiocyanato, aminocarbonyl or (C₁-C₄)alkyl, (C₁-C₄)alkoxy, (C₁-C₄)alkylthio, mono(C₁-C₄)alkylamino, di(C₁-C₄)alkylamino, (C₂-C₄)alkenyl, (C₂-C₄)alkynyl, [(C₁-C₄)alkyl]carbonyl, [(C₁-C₄)alkoxy]carbonyl, mono(C₁-C₄)alkylaminocarbonyl, di(C₁-C₄)alkylaminocarbonyl, N—(C₁-C₆)alkanoylamino or N—(C₁-C₄)alkanoyl-N—(C₁-C₄)alkylamino,
where each of the last-mentioned 13 radicals is unsubstituted or substituted by one or more radicals selected from the group consisting of halogen, hydroxyl, amino, cyano, thiocyanato, (C₁-C₄)alkoxy, (C₁-C₄)haloalkoxy, (C₁-C₄)alkylthio, mono(C₁-C₄)alkylamino, di(C₁-C₄)alkylamino, (C₃-C₆)cycloalkyl, (C₃-C₆)cycloalkylamino, [(C₁-C₄)alkyl]carbonyl, [(C₁-C₄)alkoxy]carbonyl, aminocarbonyl, mono(C₁-C₄)alkylaminocarbonyl, di(C₁-C₄)alkylaminocarbonyl, phenyl, phenoxy, phenylthio, phenylcarbonyl, heterocyclyl, heterocyclyloxy, heterocyclylthio and heterocyclylamino,
where each of the last-mentioned 8 radicals is unsubstituted or has one or more substituents selected from the group consisting of halogen, nitro, cyano, (C₁-C₄)alkyl, (C₁-C₄)alkoxy, (C₁-C₄)alkylthio, (C₁-C₄)haloalkyl, (C₁-C₄)haloalkoxy, formyl, (C₁-C₄)alkylcarbonyl and (C₁-C₄)alkoxycarbonyl,
or is (C₃-C₉)cycloalkyl, phenyl, phenoxy, phenylthio, phenylcarbonyl, heterocyclyl, heterocyclyloxy, heterocyclylthio or heterocyclylamino,
where each of the last-mentioned 9 radicals is unsubstituted or substituted by one or more radicals selected from the group consisting of halogen, hydroxyl, amino, nitro, formyl, carboxyl, cyano, thiocyanato, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, mono(C₁-C₄)alkylamino, di(C₁-C₄)alkylamino, (C₃-C₆)cycloalkyl, [(C₁-C₄)alkyl]carbonyl, [(C₁-C₄)alkoxy]carbonyl, aminocarbonyl, mono(C₁-C₄)alkylaminocarbonyl and di(C₁-C₄)alkylaminocarbonyl,
or two adjacent radicals X together are a fused-on cycle with 4 to 6 ring atoms which is carbocyclic or contains hetero ring atoms selected from the group consisting of O, S and N and which is unsubstituted or substituted by one or more radicals selected from the group consisting of halogen, (C₁-C₄)alkyl and oxo, and
n is 0, 1, 2 or 3.

7. A process for the preparation of a compound of the formula (I) or a salt thereof as defined in claim 1, which comprises
a) reacting a compound of the formula (II),
R¹-Fu (II)
where Fu is the functional group selected from the group consisting of carboxylic ester, carboxylic orthoester, carboxylic acid chloride, carboxamide, carboxylic anhydride and trichloromethyl
with a compound of the formula (III) or an acid addition salt thereof 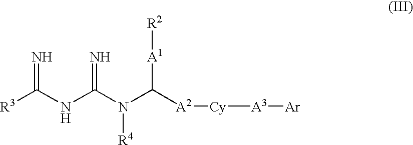
or
b) reacting a compound of the formula (IV) 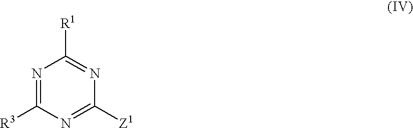
where Z¹ is an exchangeable radical or a leaving group,
with a suitable amine of the formula (V) or an acid addition salt thereof 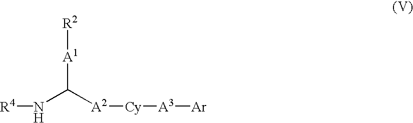
where, in formulae (II), (III), (IV) and (V), the radicals R¹, R², R³, R⁴, A¹, A², A³, Ar and Cy are as defined in formula (I).

8. A herbicidal or plant-growth-regulatory composition, which comprises one or more compounds of the formula (I) or salts thereof as claimed in claim 1 and formulation auxiliaries conventionally used in crop protection.

9. A method of controlling harmful plants or for regulating the growth of plants which comprises using a compound of the formula (I) or a salt thereof as claimed in claim 1 as herbicide or plant growth regulator.

10. A method as claimed in claim 9 of controlling harmful plants or for regulating the growth of plants, which comprises applying an effective amount of one or more compounds of the formula (I) or their salts as claimed in claim 1 to the plants, plant seeds or the area under cultivation.

11. The method as claimed in claim 10, wherein the compounds of the formula (I) or their salts are employed for controlling harmful plants or for regulating the growth in crops of useful plants or ornamentals.

12. The method as claimed in claim 11, wherein the crop plants are transgenic crop plants.

13. A compound of the formula (III) or (V), as defined in claim 7.
